(19) United States
(12) Patent Application Publication
(10) Pub. No.: US 2021/0379046 A1
(43) Pub. Date: Dec. 9, 2021

(54) Methods of Treating Diseases Using Kinase Modulators

(71) Applicant: Memorial Sloan Kettering Cancer Center, New York, NY (US)

(72) Inventors: David A. Scheinberg, New York, NY (US); Claire Y. Oh, Colonia, NJ (US); Martin Gunther Klatt, New York, NY (US)

(73) Assignee: Memorial Sloan Kettering Cancer Center, New York, NY (US)

(21) Appl. No.: 17/288,351

(22) PCT Filed: Oct. 31, 2019
(86) PCT No.: PCT/US2019/059125

§ 371 Date: Apr. 23, 2021

Related U.S. Application Data
(60) Provisional application No. 62/754,286, filed on Nov. 1, 2018.

(51) Int. Cl. A61K 31/4545; A61K 31/506; A61K 31/5377; C07K 16/28; C07K 16/32; A61P 35/00

(52) U.S. Cl. CPC A61K 31/4545; A61K 31/506; A61P 35/00; C07K 16/2833; C07K 16/32; A61K 31/5377

(57) ABSTRACT

Provided herein are methods of modulating immune response, including methods of treating a cancer or an infection using a combination of kinase modulators and immunotherapy that promotes immune response. Also provided herein are methods of treating an autoimmune disease or graft-versus-host disease, and methods of reducing the risk of solid organ transplant rejection using a combination of kinase modulators and immunosuppressive therapy.

### CROSS-REFERENCE TO RELATED APPLICATIONS

This application claims the benefit of U.S. Provisional Patent Application No. 62/754,286, filed Nov. 1, 2018, which is incorporated by reference herein in its entirety.

### GOVERNMENT RIGHTS STATEMENT

This invention was made with government support under CA055349 awarded by National Institutes of Health. The government has certain rights in the invention.

### REFERENCE TO SEQUENCE LISTING SUBMITTED ELECTRONICALLY

This application incorporates by reference a Sequence Listing submitted with this application as a text file entitled “13542-060-228_Sequence_Listing_ST25.txt” created on Oct. 30, 2019 and having a size of 5,780 bytes.

### 1. FIELD

Provided herein are methods of modulating immune response, including methods of treating a cancer or an infection using a combination of kinase modulators and immunotherapy that promotes immune response. Also provided herein are methods of treating an autoimmune disease or graft-versus-host disease, and methods of reducing the risk of solid organ transplant rejection using a combination of kinase modulators and immunosuppressive therapy.

### 2. BACKGROUND

The accumulating approvals and recent clinical successes of T cell based immunotherapies are quickly revolutionizing the approach to treating cancer. With the spotlight on emerging therapies like checkpoint blockade, CAR T cells, TCR engineered cells, and adoptive T cell transfer, the research and medical field are constantly finding ways to improve or expand these treatments. Though the mechanisms behind these therapies vary tremendously, the core interaction underlying these as well as the adaptive immune system's ability to fight off cancerous cells is the interaction between the human leukocyte antigen (HLA) and the CD8+ T cell receptor (TCR). HLA is a glycoprotein that functions in the immune system by binding to peptides of intracellular origin and displaying them on the cell surface to be surveyed by effector T cells. T cells have complementarity-determining regions (CDRs) in the TCR that engage the HLA molecule while other CDRs recognize the presented peptide (Burrows et al., 2010, Proc Natl Acad Sci USA 107(23):10608-10613). If the peptide is deemed foreign or a self-neoantigen, this triggers the release of lytic granules from the T cell, resulting in the killing of the infected or cancerous cells, respectively. Hence, this interaction between the tumor cell's HLA and the T cell's TCR is essential in producing the cytotoxic T cell response. However, the low surface presentation of tumor-associated antigens and the ability of some cancers to downregulate antigen presentation machinery hinders the ability of T cells to recognize and destroy their target (Demanet et al., 2004, Blood 103(8): 3122-3130). Multiple studies, including those performed in lung, melanoma, bladder, and colorectal carcinomas have shown up to two-thirds of the tissue samples or cell lines having at least one alteration in HLA. From loss of the entire HLA class I to defective antigen presentation machinery (like beta 2-microglobulin mutations) to loss of a specific HLA locus, cancer cells use downregulation of HLA levels as a potential mechanism of immune escape (Mcgranahan et al., 2017, Cell 171(6):1259-1271; Mendez et al., 2009, Immunotherapy 58(9):1507-1515; Cabrera et al., 2003, Tissue Antigens 62(4):324-327; and Maleno et al., 2004, Immunogenetics 56(4):244-253).

An in vitro, pooled, shRNA kinase screen was previously conducted and showed that inhibition of the mitogen-activated protein kinase (MAPK) pathway lead to increased transcript, protein, and surface levels of HLA and as a result, increased in vitro cytotoxicity of TCR mimic antibodies (Brea et al., 2016, Cancer Immunol Res 4(11): 936-947; see also International Patent Application Publication No. WO 2017/160717). The anaplastic lymphoma kinase (ALK) is a receptor tyrosine kinase that has been implicated in oncogenesis due to genetic mutations. In normal tissues, ALK is almost exclusively expressed in the central and peripheral nervous system. It is expressed in regions involved in brain development during embryogenesis and expressed in minimal levels afterwards (Wu et al., 2016, J Hematol Oncol 9:19). Full length ALK is found on the cell surface and contains an extracellular ligand-binding domain, transmembrane domain, and intracellular tyrosine kinase domain. Similar to RET, its oncogenic fusion protein product is seen in a variety of cancers. One such fusion, nucleophosmin-anaplastic lymphoma (NPM-ALK) results from the translocation between chromosome 2 and 5 and is found in approximately 75-80% of all ALK positive anaplastic lymphomas (ALCLs) (Webb et al., 2009, Expert Rev Anticancer Ther 9(3):331-356). Nucleophosmin is a ubiquitously expressed protein that shuttles ribonucleoproteins between the nucleolous and the cytoplasm hence, NPM-ALK has a characteristic nuclear and cytoplasmic subcellular localization. Homodimers or NPM/NPM-ALK heterodimers lead to constitutive activation of ALK and subsequent activation of downstream signalling pathways like MAPK and PI3K (George et al., 2014, Oncotarget 5(14):5750-5763).

Erb-b2 receptor tyrosine kinase 2 (ERBB2, also known as HER2) is another tyrosine kinase receptor that drives multiple cancers. Unlike other receptor tyrosine kinases (RTKs) in the EGFR (epidermal growth factor receptor) family, it cannot bind ligands but instead forms heterodimers or homodimers to activate (Rimawi et al., 2015, Annu Rev Med 66:111-128). Once activated, it can activate the MAPK pathway through SHC and Grb2 adaptor proteins.

Citation of a reference herein shall not be construed as an admission that such is prior art to the present disclosure.

### 3. SUMMARY OF THE INVENTION

The present invention provides methods of treating cancers or infections using a combination of kinase modulators and immunotherapy that promotes immune response. Also provided herein are methods of treating autoimmune diseases or graft-versus-host diseases, and methods of reducing the risk of solid organ transplant rejection using a combination of kinase modulators and immunosuppressive therapy.

In one aspect, provided herein is a method of treating a cancer in a patient comprising: (i) administering to the patient an inhibitor of the activity of a kinase, and (ii) administering to the patient an immunotherapy that promotes an immune response against the cancer; wherein the kinase is ALK (anaplastic lymphoma kinase). In a specific embodiment, the inhibitor is crizotinib, ceritinib, or alectinib. In a specific embodiment, the inhibitor is a small molecule inhibitor. In another specific embodiment, the inhibitor is an antibody (for example, a monoclonal antibody) or an antigen-binding fragment thereof that specifically binds to the kinase. In one embodiment, the inhibitor is administered in a subclinical amount.

In another aspect, provided herein is a method of treating a cancer in a patient comprising: (i) administering to the patient an inhibitor of the activity of a kinase, and (ii) administering to the patient an immunotherapy that promotes an immune response against the cancer; wherein the kinase is ERBB2 (erb-b2 receptor tyrosine kinase 2). In a specific embodiment, the inhibitor is trastuzumab or lapatinib. In a specific embodiment, the inhibitor is a small molecule inhibitor. In another specific embodiment, the inhibitor is an antibody (for example, a monoclonal antibody) or an antigen-binding fragment thereof that specifically binds to the kinase. In one embodiment, the inhibitor is administered in a subclinical amount.

In certain embodiments, the cancer described herein is breast cancer, lung cancer, ovary cancer, stomach cancer, pancreatic cancer, larynx cancer, esophageal cancer, testes cancer, liver cancer, parotid cancer, biliary tract cancer, colon cancer, rectum cancer, cervix cancer, uterus cancer, endometrium cancer, renal cancer, bladder cancer, prostate cancer, thyroid cancer, melanoma, or non-small cell lung cancer. In specific embodiments, the cancer described herein is lung cancer, thyroid cancer, or melanoma.

In another aspect, provided herein is a method of treating an infection in a patient comprising: (i) administering to the patient an inhibitor of the activity of a kinase, and (ii) administering to the patient an immunotherapy that promotes an immune response against the infection; wherein the kinase is ALK. In a specific embodiment, the inhibitor is crizotinib, ceritinib, or alectinib. In a specific embodiment, the inhibitor is a small molecule inhibitor. In another specific embodiment, the inhibitor is an antibody (for example, a monoclonal antibody) or an antigen-binding fragment thereof that specifically binds to the kinase. In one embodiment, the inhibitor is administered in a subclinical amount.

In another aspect, provided herein is a method of treating an infection in a patient comprising: (i) administering to the patient an inhibitor of the activity of a kinase, and (ii) administering to the patient an immunotherapy that promotes an immune response against the infection; wherein the kinase is ERBB2. In a specific embodiment, the inhibitor is trastuzumab or lapatinib. In a specific embodiment, the inhibitor is a small molecule inhibitor. In another specific embodiment, the inhibitor is an antibody (for example, a monoclonal antibody) or an antigen-binding fragment thereof that specifically binds to the kinase. In one embodiment, the inhibitor is administered in a subclinical amount.

In certain embodiments, the infection described herein is an infection with a virus, bacterium, fungus, helminth or protist. In specific embodiments, the infection described herein is an infection with a virus. In a specific embodiment, the infection described herein is an infection with herpesvirus. In another specific embodiment, the infection described herein is an infection with cytomegalovirus.

In a specific embodiment, the immunotherapy described herein is a vaccine.

In another specific embodiment, the immunotherapy described herein is an immune checkpoint blockade. In a further specific embodiment, the immune checkpoint blockade is an antibody (for example, a monoclonal antibody) or an antigen-binding fragment thereof that specifically binds to and reduces the activity of an immune checkpoint protein. In certain embodiments, the immune checkpoint blockade inhibits the activity of CTLA-4, PD-1, PD-L1, PD-L2, TIM-3, or LAG-3.

In another specific embodiment, the immunotherapy described herein is an adoptive immunotherapy. In a further specific embodiment, the immunotherapy described herein is an adoptive T cell therapy. In one embodiment, the adoptive T cell therapy is TCR (T-Cell Receptor)-engineered T cells. In another embodiment, the adoptive T cell therapy is CAR (Chimeric Antigen Receptor) T cells, wherein the antigen-binding domain of the CAR specifically binds to an antigen of the cancer.

In another specific embodiment, the immunotherapy described herein is a TCR mimic antibody.

In another specific embodiment, the immunotherapy described herein is a TCR based construct that encodes a soluble protein comprising the antigen recognition domain of a TCR.

In another specific embodiment, the immunotherapy described herein is an interferon, an anti-CD47 antibody, a SIRP alpha antagonist, an HDAC inhibitor, a cytokine, a TLR (Toll-Like Receptor) agonist, or an epigenetic modulator that upregulates the expression of one or more MHCs (Major Histocompatibility Complexes) or upregulates antigen presentation. In one embodiment, the immunotherapy is an epigenetic modulator that upregulates the expression of one or more MHCs or upregulates antigen presentation that is a hypomethylating agent. In another embodiment, the immunotherapy is a hypomethylating agent that is azacytidine or decitabine. In another embodiment, the immunotherapy is an interferon that is interferon alpha or interferon gamma. In another embodiment, the immunotherapy is a cytokine that is IL2 (Interleukin-2), TNF (Tumor Necrosis Factor), interferon alpha or interferon gamma. In another embodiment, the immunotherapy is a TLR agonist that is a dsDNA (double-stranded DNA) TLR agonist. In another embodiment, the immunotherapy is a TLR agonist that is a dsRNA (double-stranded RNA) TLR agonist. In another embodiment, the immunotherapy is a dsRNA TLR agonist that is polyinosinic-polycytidylic acid (poly(I:C)).

In another aspect, provided herein is a method of generating a population of antigen-presenting cells for therapeutic administration to a patient having a cancer, comprising culturing antigen-presenting cells that are loaded with or genetically engineered to express one or more immunogenic peptides or proteins derived from one or more antigens of the cancer in the presence of an inhibitor of the activity of a kinase, wherein the kinase is ALK. Also provided herein is a method of treating a cancer in a patient comprising generating a population of antigen-presenting cells according to a method of generating a population of antigen-presenting cells of this aspect, and administering to the patient the population of antigen-presenting cells. In a specific embodiment, the inhibitor is crizotinib, ceritinib, or alectinib. In a specific embodiment, the inhibitor is a small molecule inhibitor. In another specific embodiment, the inhibitor is an antibody (for example, a monoclonal antibody) or an antigen-binding fragment thereof that specifically binds to the kinase.

In another aspect, provided herein is a method of generating a population of antigen-presenting cells for therapeutic administration to a patient having a cancer, comprising culturing antigen-presenting cells that are loaded with or genetically engineered to express one or more immunogenic peptides or proteins derived from one or more antigens of the cancer in the presence of an inhibitor of the activity of a kinase, wherein the kinase is ERBB2. Also provided herein is a method of treating a cancer in a patient comprising generating a population of antigen-presenting cells according to a method of generating a population of antigen-presenting cells of this aspect, and administering to the patient the population of antigen-presenting cells. In a specific embodiment, the inhibitor is trastuzumab or lapatinib. In a specific embodiment, the inhibitor is a small molecule inhibitor. In another specific embodiment, the inhibitor is an antibody (for example, a monoclonal antibody) or an antigen-binding fragment thereof that specifically binds to the kinase.

In certain embodiments, the cancer described herein is breast cancer, lung cancer, ovary cancer, stomach cancer, pancreatic cancer, larynx cancer, esophageal cancer, testes cancer, liver cancer, parotid cancer, biliary tract cancer, colon cancer, rectum cancer, cervix cancer, uterus cancer, endometrium cancer, renal cancer, bladder cancer, prostate cancer, thyroid cancer, melanoma, or non-small cell lung cancer. In specific embodiments, the cancer described herein is lung cancer, thyroid cancer, or melanoma.

In another aspect, provided here is a method of generating a population of antigen-presenting cells for therapeutic administration to a patient having an infection, comprising culturing antigen-presenting cells that are loaded with or genetically engineered to express one or more immunogenic peptides or proteins derived from one or more antigens of the pathogen causing the infection in the presence of an inhibitor of the activity of a kinase, wherein the kinase is ALK. Also provided herein is a method of treating an infection in a patient comprising generating a population of antigen-presenting cells according to a method of generating a population of antigen-presenting cells of this aspect, and administering to the patient the population of antigen-presenting cells. In a specific embodiment, the inhibitor is crizotinib, ceritinib, or alectinib. In a specific embodiment, the inhibitor is a small molecule inhibitor. In another specific embodiment, the inhibitor is an antibody (for example, a monoclonal antibody) or an antigen-binding fragment thereof that specifically binds to the kinase.

In another aspect, provided herein is a method of generating a population of antigen-presenting cells for therapeutic administration to a patient having an infection, comprising culturing antigen-presenting cells that are loaded with or genetically engineered to express one or more immunogenic peptides or proteins derived from one or more antigens of the pathogen causing the infection in the presence of an inhibitor of the activity of a kinase, wherein the kinase is ERBB2. Also provided herein is a method of treating an infection in a patient comprising generating a population of antigen-presenting cells according to a method of generating a population of antigen-presenting cells of this aspect, and administering to the patient the population of antigen-presenting cells. In a specific embodiment, the inhibitor is trastuzumab or lapatinib. In a specific embodiment, the inhibitor is a small molecule inhibitor. In another specific embodiment, the inhibitor is an antibody (for example, a monoclonal antibody) or an antigen-binding fragment thereof that specifically binds to the kinase.

In certain embodiments, the infection described herein is an infection with a virus, bacterium, fungus, helminth or protist. In specific embodiments, the infection described herein is an infection with a virus. In a specific embodiment, the infection described herein is an infection with herpesvirus. In another specific embodiment, the infection described herein is an infection with cytomegalovirus.

In another aspect, provided herein is a method of treating an autoimmune disease in a patient comprising: (i) administering to the patient an activator of the activity of a kinase, and optionally (ii) administering to the patient an immunosuppressive therapy that suppresses the immune response associated with the autoimmune disease; wherein the kinase is ALK. In a specific embodiment, the activator is administered in a subclinical amount. In a specific embodiment, the activator is a soluble ligand of the kinase, or a soluble ligand of a receptor that activates the kinase in vivo. In another specific embodiment, the activator is an antibody (for example, a monoclonal antibody) or an antigen-binding fragment thereof that specifically binds to the kinase.

In another aspect, provided herein is a method of treating an autoimmune disease in a patient comprising: (i) administering to the patient an activator of the activity of a kinase, and optionally (ii) administering to the patient an immunosuppressive therapy that suppresses the immune response associated with the autoimmune disease; wherein the kinase is ERBB2. In a specific embodiment, the activator is administered in a subclinical amount. In a specific embodiment, the activator is a soluble ligand of the kinase, or a soluble ligand of a receptor that activates the kinase in vivo. In another specific embodiment, the activator is an antibody (for example, a monoclonal antibody) or an antigen-binding fragment thereof that specifically binds to the kinase.

In certain embodiments, the autoimmune disease described herein is multiple sclerosis, type 1 diabetes, ankylosing spondylitis, or Hashimoto's thyroiditis.

In another aspect, provided herein is a method of treating graft-versus-host disease (GvHD) in a patient comprising: (i) administering to the patient an activator of the activity of a kinase, and optionally (ii) administering to the patient an immunosuppressive therapy that suppresses the immune response associated with the GvHD; wherein the kinase is ALK. In a specific embodiment, the activator is administered in a subclinical amount. In a specific embodiment, the activator is a soluble ligand of the kinase, or a soluble ligand of a receptor that activates the kinase in vivo. In another specific embodiment, the activator is an antibody (for example, a monoclonal antibody) or an antigen-binding fragment thereof that specifically binds to the kinase.

In another aspect, provided herein is a method of treating GvHD in a patient comprising: (i) administering to the patient an activator of the activity of a kinase, and optionally (ii) administering to the patient an immunosuppressive therapy that suppresses the immune response associated with the GvHD; wherein the kinase is ERBB2. In a specific embodiment, the activator is administered in a subclinical amount. In a specific embodiment, the activator is a soluble ligand of the kinase, or a soluble ligand of a receptor that activates the kinase in vivo. In another specific embodiment, the activator is an antibody (for example, a monoclonal antibody) or an antigen-binding fragment thereof that specifically binds to the kinase.

In a specific embodiment, the GvHD described herein is an acute GvHD. In another specific embodiment, the GvHD described herein is a chronic GvHD.

In another aspect, provided herein is a method of reducing the risk of solid organ transplant rejection in a patient comprising: (i) administering to the patient an activator of the activity of a kinase, and optionally (ii) administering to the patient an immunosuppressive therapy that suppresses the immune response against the solid organ transplant; wherein the kinase is ALK. In a specific embodiment, the activator is administered in a subclinical amount. In a specific embodiment, the activator is a soluble ligand of the kinase, or a soluble ligand of a receptor that activates the kinase in vivo. In another specific embodiment, the activator is an antibody (for example, a monoclonal antibody) or an antigen-binding fragment thereof that specifically binds to the kinase.

In another aspect, provided herein is a method of reducing the risk of solid organ transplant rejection in a patient comprising: (i) administering to the patient an activator of the activity of a kinase, and optionally (ii) administering to the patient an immunosuppressive therapy that suppresses the immune response against the solid organ transplant; wherein the kinase is ERBB2. In a specific embodiment, the activator is administered in a subclinical amount. In a specific embodiment, the activator is a soluble ligand of the kinase, or a soluble ligand of a receptor that activates the kinase in vivo. In another specific embodiment, the activator is an antibody (for example, a monoclonal antibody) or an antigen-binding fragment thereof that specifically binds to the kinase.

In a specific embodiment, the immunosuppressive therapy described herein is sirolimus, everolimus, rapamycin, one or more steroids, cyclosporine, cyclophosphamide, azathioprine, mercaptopurine, fluorouracil, fludarabine, interferon beta, a TNF decoy receptor, a TNF antibody, methotrexate, a T-cell antibody, an anti-CD20 antibody, a complement inhibitor, an anti-IL6 antibody, an anti-IL2R antibody, anti-thymocyte globulin, fingolimod, mycophenolate, or a combination thereof. In one embodiment, the immunosuppressive therapy is a TNF decoy receptor that is etanercept. In another embodiment, the immunosuppressive therapy is a TNF antibody that is infliximab. In another embodiment, the immunosuppressive therapy is a T-cell antibody that is an anti-CD3 antibody (for example, OKT3). In another embodiment, the immunosuppressive therapy is an anti-CD20 antibody that is rituximab. In another embodiment, the immunosuppressive therapy is a complement inhibitor that is eculizumab. In another embodiment, the immunosuppressive therapy is an anti-IL2R antibody that is daclizumab.

In a preferred embodiment, the patient is a human patient.

### 4. BRIEF DESCRIPTION OF FIGURES

FIG. 1: ALK inhibition leads to decreased pERK levels and increased cell surface HLA levels in ALK mutated cell lines. (A) Karpas 299 and (B) SUDHL-1 cells were treated with increasing concentrations of crizotinib for 3 hrs and pERK levels were measured by western blot. After 72 hrs of crizotinib treatment, flow cytometry measured cell surface HLA-ABC on (C) Karpas 299 and (D) SUDHL-1 cells to show increases in cell surface HLA. Western blot of (E) Karpas 299 and (F) SUDHL-1 cells treated with the second-generation ALK inhibitor, ceritinib, for 3 hrs. Flow cytometry analysis of (G) Karpas 299 and (H) SUDHL-1 cells treated with ceritinib for 72 hrs.

FIG. 2: Alectinib shuts down pERK expression in lysates of (A) Karpas 299 and (B) SUDHL-1 treated with inhibitor for 3 hrs. Alectinib upregulates cell surface HLA in (C) Karpas 299 and (D) SUDHL-1 cells at 72 hrs. (E) Crizotinib does not decrease pERK levels in a EML4-ALK cell line H2228, hence (F) no increase in surface HLA. Ceritinib decreases pERK levels very slightly and have a corresponding slight increase in HLA. At higher doses of drug tested, the cells did not survive.

FIG. 3: Surface HLA-A,B,C increase with RET inhibition in TPC1 cells, using (A) AST487, and (B) cabozantinib. (C) Treatment with siRNAs against RET for 96 hrs increases surface HLA-A*02 and HLA-A,B,C compared to the control (scrambled siRNA) in TPC1 cells. AST487 treatment for 72 hrs leads to increases in surface HLA in two other RET mutant cell lines, (D) TT cells (a medullary thyroid carcinoma cell line with a point mutation in codon 634 of RET leading to a cysteine to tryptophan substitution) and (E) LC-2/ad (a lung adenocarcinoma harboring the CCDCl6-RET fusion). Additional validation with two other RET inhibitors, (F) CEP-32496 and (G) cabozantinib.

FIG. 4: RET inhibition in TPC1 cells also leads to decreased pERK levels and increased levels of HLA. TPC1 cells, a papillary thyroid carcinoma line with a RET/PTC1 rearrangement, were treated with the RET inhibitor, AST487. (A) After 72 hrs, a dose dependent increase in surface HLA-A*02 was measured through flow cytometry. (B) Phospho-ERK levels decreased with AST487 treatment. Similar results were observed with one other RET inhibitor. Cabozantinib (C) increased surface HLA and (D) decreased pERK expression.

FIG. 5: (A) Histogram from flow cytometry showing slight binding of ESK to untreated TPC1 over isotype. (B) Cell surface HLA-A,B,C expression of tumors isolated from NRG mice subcutaneously injected with Karpas 299 and treated with crizotinib for 7 days through oral gavage. (C) PD-L1 levels decrease in vivo with crizotinib treatment.

FIG. 6: Therapeutic utility of RET inhibition. (A) ESK, a TCR mimic antibody, was fluorescently labeled to probe binding through flow cytometry. Increased binding of ESK was seen with AST487 treatment. (B) Chromium-51 labeled TPC1 cells were incubated with ESK and human PBMCs for 5 hrs at 32° C. and percent specific lysis was calculated for DMSO (control), trametinib, and AST487 groups by measuring chromium levels in the media. Increased HLA and ESK binding with RET inhibition lead to increased in vitro ADCC cytotoxicity. (C) TPC1 cells were subcutaneously injected into NRG mice and harvested after 7 days of control, 10 mg/kg, or 35 mg/kg AST487 treatment through oral gavage. HLA-A*02 and HLA-A,B,C increased in a dose related manner in cells treated with AST487. (D) PD-L1 levels did not change in vivo.

FIG. 7: Mechanism of HLA increase is transcript level-based. (A) Western blots probing for HLA-A and beta-2-microglobulin show increase in protein levels at 72 hrs of RET inhibitor treatments. (B) qPCR of RNA extracted at 48 hrs show increase in HLA and antigen processing machinery with RET inhibitor treatments. (C) Similarly, western blots of Karpas 299 and SUDHL-1 after 72 hrs of ceritinib treatment show increase in HLA-A and beta-2-microglobulin. (D) Increase in RNA levels of HLA and antigen processing machinery is seen after certinib treatment.

FIG. 8: (A) qPCR of ALCL lines treated with crizotinib for 48 hrs show increase in HLA and antigen processing machinery transcript levels. (B) Western blots at 72 hrs show increase in HLA and beta-2microglobulin protein. Alectinib treatment on ALCL lines show similar increases in (C) transcript levels and (D) protein levels.

FIG. 9: STAT3 may play a role in HLA regulation in TPC1 cells. (A) TPC1 cells were treated with the same doses of AST487 and trametinib for 24 hrs and cells were lysed and western blots were run. Blots were imaged at the same time for the same exposure to accurately compare the levels of pERK. Trametinib was more effective in shutting down pERK at lower doses. (B) Flow cytometry of TPC1 cells treated with AST487 and trametinib for 72 hrs. Experiments were run on different days, but normalized to their own DMSO treated groups. For the same concentrations, AST487 had higher levels of HLA upregulation. (C) Time course of TPC1 cells treated with 10 nM AST487 shows increased phosphorylation of STAT3 at 24 hrs. (D) TPC1 cells were pretreated with siRNAs against STAT3 or a scrambled sequence (control). After 24 hrs, cells were treated with DMSO (control), 10 nM AST487, or IFN gamma (positive control) for 72 hrs and surface HLA-A*02 was measured through flow cytometry.

FIG. 10: (A) BRAF V600E mutation in TPC1 hinders HLA upregulation with RET inhibitor but does not suppress. (B) Pre-treatment of siSTAT3 for 24 hrs before RET inhibition causes increased beta-2-microglobulin and HLA transcript levels. (C) Time course of TPC1 cells treated with siScramble or siSTAT3 for 24 hrs.

FIG. 11: Mass spectrometry of eluted presented peptides shows a change in peptide repertoire after RET inhibition. (A) W6/32, an antibody that binds to HLA-A,B,C, was coupled to an activated Sepharose-CNBr column. Lysate from TPC1 cells treated with DMSO (control), 10 nM AST487, or 100 nM cabozantinib were run through the column and HLA-peptide complexes were bound. Peptides were eluted and collected for mass spectrometry. The results from three runs were pooled. The DMSO treated group had 4211 unique ligands compared to the 5274 and 4850 unique ligands from the AST487 and cabozantinib treatments, respectively. The 15-25% increase in unique peptides indicate that RET inhibition can alter peptide presentation on HLA. The approximately 1818 and 1642 new peptides that arise in each RET inhibitor groups indicate that new targets can arise after small molecule treatment allowing for additional targeted therapy. Network analysis of the (B) peptides only from the RET inhibitor overlap and (C) all new peptides from RET inhibitor treated samples show a convergence on the pathways involved in negative regulation of the cell cycle and cell cycle arrest.

FIG. 12: (A) Overlap and (B) quantification of eluted peptides in Karpas 299 cells treated with DMSO, 100 nM crizotinib, or 100 nM ceritinib.

FIG. 13: (A)-(D) Vemurafenib, a BRAF (B-Raf proto-oncogene, serine/threonine kinase) inhibitor, did not upregulate HLA in BRAF mutant myeloma cell lines.

FIG. 14: (A)-(D) Trastuzumab, an ERBB2 (erb-b2 receptor tyrosine kinase 2) inhibitor, decreased pERK in SKOV3 and not A498 cells, hence HLA upregulation was only seen in SKOV3 cells.

FIG. 15: Lapatinib, an ERBB2 inhibitor, upregulated HLA in SKOV3 cells.

FIG. 16: (A-B) Surface HLA on trastuzumab treated SKOV3 cells could potentially be limited by beta-2-microglobulin protein.

FIG. 17: ALK inhibition decreased pERK levels and increased surface HLA levels in ALK mutated cell lines. (A) Karpas 299 cells were treated with increasing concentrations of crizotinib for 3 hrs and pERK and ERK (loading control) were measured by western blot. (B) After 72 hrs of crizotinib treatment, flow cytometry was used to measure cell surface HLA-A, B, C on Karpas 299 cells. Similarly, SUDHL-1 cells were treated with crizotinib and (C) pERK and ERK and (D) cell surface HLA molecules were measured. Western blot for ERK and pERK on (E) Karpas 299 cells and (F) SUDHL-1 cells treated with the second-generation ALK inhibitor, ceritinib, for 3 hrs. Flow cytometry analysis of HLA-A, B, C expression in (G) Karpas 299 cells and (H) SUDHL-1 cells treated with ceritinib for 72 hrs. W6/32-APC antibody was used to measure HLA-A,B,C. P values were calculated with GraphPad Prism 7 using an unpaired t test for flow cytometry experiments. Error bars indicate SD for flow cytometry. All flow cytometry experiments were performed in technical triplicates and with a minimum of 2 biological replicates. Western blots were done at least two to three times. Representative demonstration blots are shown only. Values are reported in figures with “*” equal to P≤0.05, “**” equal to P≤0.01, “***” equal to P≤0.001, and “****” equal to P≤0.0001. No symbol indicates not statistically significant (P>0.05).

FIG. 18: Increase in cell surface HLA depends on decrease in pERK in ALK inhibited ALCL cells. Alectinib shut down pERK expression in lysates of (A) Karpas 299 and (B) SUDHL-1 treated with inhibitor for 3 hrs. Alectinib upregulates cell surface HLA in (C) Karpas 299 and (D) SUDHL-1 cells at 72 hrs. (E) Time course of HLA levels in Karpas cells treated with ALK inhibitors at day 0. At 4 and 6 days, cells showed upregulation of HLA. After that. time course was stopped because control cells were too dense. (F) Representative flow histograms for ALK inhibition. (G) Crizotinib does not decrease pERK levels in a EML4-ALK cell line H2228, hence (H) no increase in surface HLA was seen. Ceritinib decreased pERK levels very slightly and resulted in a corresponding slight increase in HLA. At higher doses of drug tested, the cells did not survive. All flow cytometry was performed in triplicate. HLA-A,B,C was measured by the W6/32 antibody.

FIG. 19: RET inhibition led to increased HLA. Surface HLA-A,B,C after 72 hrs of RET inhibition in TPC1 cells, using (A) AST487 and (B) cabozantinib. (C) TPC1 surface HLA-A*02 and HLA-A,B,C after treatment with RET siRNAs after 96 hrs. HLA was measured after AST487 treatment for 72 hrs in two other RET mutant cell lines (changes are significant p<0.05-0.001), (D) TT cells (a medullary thyroid carcinoma cell line with a point mutation in codon 634 of RET leading to a cysteine to tryptophan substitution) and (E) LC-2/ad (a lung adenocarcinoma harboring the CCDCl6-RET fusion). Small changes in cell surface HLA with treatment of two other RET inhibitors, (F) CEP-32496 and (G) cabozantinib. All flow cytometry was performed in triplicate. HLA-A,B,C was measured by the W6/32 antibody. HLA-A*02 was measured by the BB7 antibody.

FIG. 20: RET inhibition in TPC1 cells led to decreased pERK levels and increased surface expression of HLA. TPC1 cells, a papillary thyroid carcinoma line with a RET/PTC1 rearrangement, were treated with the RET inhibitor, AST487. (A) After 72 hrs, cell surface HLA-A*02 was measured through flow cytometry. (B) pERK and ERK (loading control) were measured at 24 hrs by western blot. Similar results were observed with another RET inhibitor: cabozantinib. (C) Cell surface HLA expression and (D) pERK and ERK expression after Cabozantinib treatment. BB7-APC antibody was used to measure HLA-A*02. P values were calculated with GraphPad Prism 7 using an unpaired t test for flow cytometry experiments. Error bars indicate standard deviation (SD) for flow cytometry. All flow cytometry experiments were performed in technical triplicates and with a minimum of 2 biological replicates. Western blots were done at least two to three times. Representative demonstration blots are shown only. Values are reported in figures with “*” equal to P≤0.05, “**” equal to P≤0.01, “***” equal to P≤0.001, and “****” equal to P≤0.0001. No symbol indicates not statistically significant (P>0.05).

FIG. 21: The regulation of HLA increase was at the transcript level. (A) Representative western blots probing for HLA-A, beta-2-microglobulin (B2M), and GAPDH (loading control) in TPC1 cells at 72 hrs after RET inhibitor treatments. (B) HLA and antigen processing machinery (TAP1, TAP2 and Beta-2 microglobulin) transcript levels measured by qPCR at 48 hrs after RET inhibitor treatment. (C) Western blots for HLA-A, beta-2-microglobulin (B2M), and GAPDH (loading control) and (D) RNA levels of HLA-A, beta-2-microglobulin (B2M), and TAP-1 and TAP-2 in Karpas 299 cells and SUDHL-1 cells after ceritinib treatment. qPCR experiments were performed in technical triplicate. Error bars indicate SEM. All experiments were performed in technical triplicates and with a minimum of 2 biological replicates. Western blots were done at least two to three times. Representative demonstration blots are shown only. Values are reported in figures with “*” equal to P≤0.05, “**” equal to P≤0.01, “***” equal to P≤0.001, and “****” equal to P≤0.0001. No symbol indicates not statistically significant (P>0.05).

FIG. 22: Increase in antigen processing machinery transcript and protein in ALK inhibited ALCL cells. (A) qPCR of ALCL lines treated with crizotinib for 48 hrs showed increases in HLA and antigen processing machinery transcript levels. (B) Western blots at 72 hrs showed increases in HLA and beta-2microglobulin protein. Alectinib treatment on two ALCL lines showed similar increases in (C) transcript levels and (D) protein levels. qPCR assays were performed in technical triplicate.

FIG. 23: (A) Karpas299 and TPC1 cells were treated with DMSO, Alectinib (100 nM) or AST 487 (10 nM), respectively, alongside various concentrations of ruxolitinib (RUX) for 72 hours, as indicated in the inset legend. Cells were harvested and stained with anti-HLA-A02 antibody (BB7-FITC) for flow cytometry. The top panel shows Karpas299 cells. Alectinib treatment led to upregulation of HLA, which was unaffected by any concentration of RUX. The bottom panel shows TPC1 cells. AST 487 led to upregulation of HLA, which was unaffected by any concentration of RUX. (B) Karpas299 and TPC1 cells were treated with DMSO, IFN-γ, or IFN-γ and RUX for 72 hours. Phosphorylated STAT1 (pSTAT1) was upregulated in both cell lines in response to IFN-γ treatment. Ruxolitinib inhibited pSTAT1 induction in both cell lines, confirming inhibitory action on JAK signaling.

FIG. 24: Karpas299 and TPC1 cells were treated with DMSO vehicle, 100 nm alectinib (Alec) or 10 nm AST487 (AST), respectively, for 48 and 72 hrs, as indicated by the inset legend. Supernatant media were harvested after treatment and analyzed by the Luminex device (Luminex Corporation, Austin, Tex.) for relevant cytokine secretion. The left column shows data from Karpas299 and the right column is from TPC1 cells. On the left side of each panel is data after 24 hours. On the right side of each panel is data after 48 hours. Cytokines measured are noted on the top of each panel and displayed on the y axis in pg/ml. Alectinib inhibition in Karpas299 lymphoma had no effect on IFNα, IFNγ, IL4, and reduced IL6 and TNFα secretion. AST487 inhibition in TPC1 thyroid cells had no effect on IFNα, IFNγ, IL4, IL6 and TNFα secretion. Therefore, the ALK and RET inhibitors do not appear to act to upregulate the JAK/STAT pathway indirectly by increased cytokine release. As a positive control, IFNγ increased both IL4 and IL6 in these cells, which was reduced by 1000 nm ruxilitinib (Rux). IFNγ detected in the Luminex assay (50-70 ng/ml) is from the added IFNγ at 100 ng/ml at time zero. Therefore, the increase in HLA after inhibitor treatment in these cell lines is not due to indirect activation of the JAK/STAT pathway via autocrine cytokine signaling. The data points shown in this figure are in pairs for each condition. The data point pairs for each time point are, from left to right: DMSO, Rux, Alec, IFNg, and IFNg+Rux.

FIG. 25: Increased tumoral surface HLA expression and decreased tumoral PD-L1 expression in vivo during ALK inhibition. (A) TPC1 cells were subcutaneously injected into NRG mice and harvested after 7 days of AST487 treatment or vehicle treatment (n=5). Cell surface HLA-A*02:01 and HLA-A, B, C were measured (with BB7 and W6/32 respectively). (B) PD-L1 levels were measured after RET inhibition. (C, D) Karpas 299 cells were subcutaneously injected into NSG mice, treated with alectinib, and harvested (n=5). HLA-A, B, C and PD-L1 levels were measured by flow cytometry. P values were calculated with GraphPad Prism 7 using an unpaired t test for flow cytometry experiments. Error bars indicate SD for flow cytometry. All flow cytometry experiments were performed in technical triplicates and with a minimum of 2 biological replicates. Values are reported in figures with “*” equal to P≤0.05, “**” equal to P≤0.01, “***” equal to P≤0.001, and “****” equal to P≤0.0001. No symbol indicates not statistically significant (P>0.05).

FIG. 26: ALK inhibition in vivo and PD-L1 levels in vitro. (A) Cell surface HLA-A,B,C expression of tumors isolated from NRG mice (n=5) subcutaneously injected with Karpas 299 and treated with crizotinib for 7 days through oral gavage. (B) PD-L1 levels decreased in vivo with crizotinib treatment of Karpas 299. PD-L1 levels after alectinib treatment in vitro in (C) Karpas 299 and (D) SUP-M2 (performed in triplicate).

FIG. 27: Checkpoint ligands levels in response to ALK and RET inhibition. (A,B) Surface levels of nectin-2 and galectin-9 were measured after ALK inhibition by alectinib and crizotinib in Karpas 299 cells. (C,D) Surface levels of nectin-2 and galectin-9 were measured after RET inhibition by AST487 and cabozantinib in TPC1 cells.

FIG. 28: Mass spectrometry of eluted HLA class I presented peptides showed a change in peptide number and repertoire after RET inhibition. (A) HLA bound peptides from lysate of TPC1 cells treated with DMSO (control), 10 nM AST487, or 100 nM cabozantinib were analyzed by mass spectrometry (n=3). Only peptides found in all three separate runs were counted. Each circle encompasses the unique peptides identified after treatment; overlaps of circles show presence of each peptide in 2 or more groups. 458 and 492 new peptides appeared in each of the two RET inhibitor groups, respectively. (B) IFN-gamma ELISpot data for T cells stimulated with newly arising peptides after RET inhibitor treatment (TLSGHSQEV (SEQ ID NO:2), VYSLIKNKI (SEQ ID NO:3), SYNEHWNYL (SEQ ID NO:4), ALSGLAVRL (SEQ ID NO:5)). A representative figure is shown of two. PHA was used as a positive control. An irrelevant peptide (GRKPPLLKK (SEQ ID NO:9)) and CD14+ cells were used as negative controls.

FIG. 29: Analysis of peptide repertoire changes after RET inhibition. (A) Profile of eluted peptides found at least once in TPC1 cells treated with DMSO (control), 10 nM AST487, or 100 nM cabozantinib (n=3). (B) Profile of eluted peptides in Karpas 299 cells treated with DMSO, 100 nM crizotinib, or 100 nM ceritinib. (C) Comparison of motifs of all A*02 9-mer peptides eluted from mass spectrometry after control or RET inhibitor treatments. RNA-Seq data shows genes upregulated at least two-fold compared to control with (D) AST487 and (E) cabozantinib treatment. (F) Table of genes that had at least a two-fold increase in gene expression and whose peptides were detected in mass spectrometry. (G) Peptides found in at least 1 run in the control group compared to peptides found in all 3 runs in the RET inhibitor groups.

FIG. 30: PBMC viability and HLA levels were not affected by inhibitors. (A) PBMCs were isolated and viability was measured with PI staining after incubation with ALK inhibitors, RET inhibitors, or control (DMSO). Different cell populations were gated and viability is displayed. (B) Flow histogram showing inhibitors did not affect HLA levels of isolated T cells.

FIG. 31: HLA-E levels did not change with drug treatment. TPC1 cells were treated with AST487 and cabozantinib and surface HLA-E levels were measured after 72 hrs. Similarly, Karpas 299 cells were treated with alectinib and crizotinib, and HLA-E levels were measured.

FIG. 32: Unmasked antigen led to lysis of TPC1 cells by a TCR mimic antibody. (A) ESK1, a TCR mimic antibody, was fluorescently labeled to probe binding after 72 hours RET inhibitor treatment by flow cytometry. (B) Chromium-51 labeled TPC1 cells were incubated with ESK1 and human PBMCs for 5 hrs at 37° C. and percent specific lysis was calculated for DMSO (control) and AST487 treated groups. P values were calculated with GraphPad Prism 7 using an unpaired t test for flow cytometry experiments. Error bars indicate SD for flow cytometry. All flow cytometry experiments were performed in technical triplicates and with a minimum of 2 biological replicates. Values are reported in figures with “*” equal to P≤0.05, “**” equal to P≤0.01, “***” equal to P≤0.001, and “****” equal to P≤0.0001. No symbol indicates not statistically significant (P>0.05). Chromium release was done twice. One plot is shown.

FIG. 33: Simplified schema of signaling pathway for HLA upregulation. MEK, ALK or RET positively regulate the output of the MAPK pathway, which in turn downregulates STAT1, which leads to reduced HLA. Inhibitors of these kinases, reverse the process.

FIG. 34: Summary of the inhibitors effects on HLA, antigen processing machinery, and checkpoint ligands.

### 5. DETAILED DESCRIPTION

The present invention provides methods of regulating processes involving presentation of peptides by class I MHC (in humans, HLA). The present invention provides methods of treating a cancer, an infection, an autoimmune disease, and graft-versus-host disease (GvHD), respectively, using kinase modulators, and methods of reducing the risk of solid organ transplant rejection using modulators of specific kinases. In a preferred embodiment, the kinase is anaplastic lymphoma kinase (ALK). In a specific embodiment, the kinase is erb-b2 receptor tyrosine kinase 2 (ERBB2). The invention identifies ALK and ERBB2 as kinases that are negative regulators of class I MHC gene expression. An inhibitor of a kinase selected from ALK and ERBB2 can be used, preferably in combination with immune-promoting immunotherapy, to increase an immune response where such is desired, ex vivo, or in vivo (by administration to a patient), e.g., to treat cancer, viral infection, etc. An activator of a kinase selected from ALK and ERBB2 can be used, preferably in combination with immunosuppressive therapy, to suppress an immune response where such is desired, ex vivo, or in vivo (by administration to a patient), e.g., to treat autoimmune disease, GvHD, or to reduce the risk of solid organ transplant rejection, etc.

The inhibitor of a kinase used in the methods of the invention decreases or blocks the activity of the kinase. The activator of a kinase used in the methods of the invention increases or initiates the activity of the kinase.

5.1. Treatment of Cancer

In one aspect, provided herein are methods of treating a cancer in a patient comprising: (i) administering to the patient an inhibitor of the activity of a kinase, and (ii) administering to the patient an immunotherapy that promotes an immune response against the cancer; wherein the kinase is ALK.

In another aspect, provided herein are methods of treating a cancer in a patient comprising: (i) administering to the patient an inhibitor of the activity of a kinase, and (ii) administering to the patient an immunotherapy that promotes an immune response against the cancer; wherein the kinase is ERBB2.

According to the invention, and without intending to be bound by a mechanism, inhibition of the activity of a kinase that is ALK or ERBB2 upregulates class I MHC gene expression on cancer cells (in human patients, such inhibition upregulates HLA-A expression, and preferably also upregulates HLA-B expression and HLA-C expression on cancer cells). In various embodiments, the inhibitor is administered in a subclinical amount. A subclinical amount of the inhibitor refers to an amount of the inhibitor at which no clinical effect or less than optimal clinical effect is detected when the inhibitor is administered alone (i.e., not in combination with the immunotherapy). In specific embodiments, the subclinical amount is lower than the amount of the inhibitor commonly used in the standard-of-care therapy for the cancer to be treated. In specific embodiments wherein the inhibitor is FDA (Food and Drug Administration)-approved for treating the cancer, the subclinical amount is lower than the FDA-approved amount for treating the cancer.

In another aspect, provided herein are methods of generating a population of antigen-presenting cells for therapeutic administration to a patient having a cancer, comprising culturing antigen-presenting cells that are loaded with or genetically engineered to express one or more immunogenic peptides or proteins derived from one or more antigens of the cancer in the presence of an inhibitor of the activity of a kinase, wherein the kinase is ALK. In another aspect, provided herein are methods of generating a population of antigen-presenting cells for therapeutic administration to a patient having a cancer, comprising culturing antigen-presenting cells that are loaded with or genetically engineered to express one or more immunogenic peptides or proteins derived from one or more antigens of the cancer in the presence of an inhibitor of the activity of a kinase, wherein the kinase is ERBB2. In another aspect, provided herein are methods of treating a cancer in a patient comprising generating a population of antigen-presenting cells according to methods described herein and administering to the patient the population of antigen-presenting. The antigen-presenting cells can be, for example, dendritic cells, cytokine-activated monocytes, or PBMCs. Preferably, the antigen-presenting cells are dendritic cells. Preferably, the antigen-presenting cells are autologous to the human patient (e.g., dendritic cells autologous to the human patient).

In another embodiment, provided herein are ex vivo methods of generating a population of antigen-specific T cells for therapeutic administration to a patient having a cancer, comprising co-culturing T cells with antigen-presenting cells that are loaded with or genetically engineered to express one or more immunogenic peptides or proteins derived from one or more antigens of the cancer in the presence of an inhibitor of the activity of a kinase, wherein the kinase is ALK. In another embodiment, provided herein are ex vivo methods of generating a population of antigen-specific T cells for therapeutic administration to a patient having a cancer, comprising co-culturing T cells with antigen-presenting cells that are loaded with or genetically engineered to express one or more immunogenic peptides or proteins derived from one or more antigens of the cancer in the presence of an inhibitor of the activity of a kinase, wherein the kinase is ERBB2. In another aspect, provided herein are ex vivo methods of treating a cancer in a patient comprising generating a population of antigen-specific T cells according to methods described herein and administering to the patient the population of antigen-specific T cells. The antigen-presenting cells can be, for example, dendritic cells, cytokine-activated monocytes, or PBMCs. Preferably, the antigen-presenting cells are dendritic cells. Preferably, the antigen-presenting cells are autologous to the human patient (e.g., dendritic cells autologous to the human patient).

While the methods of treating a cancer described in this disclosure are largely methods of combination therapy, the present invention also contemplates monotherapies using kinase inhibitors alone to treat cancer. Therefore, in another aspect, provided herein are methods of treating a cancer in a patient comprising administering to the patient an inhibitor of the activity of a kinase, wherein the kinase is ALK; and in another aspect, provided herein are methods of treating a cancer in a patient comprising administering to the patient an inhibitor of the activity of a kinase, wherein the kinase is ERBB2.

Inhibitors of the activity of a kinase that is ALK and inhibitors of the activity of a kinase that is ERBB2, that can be employed in the methods described herein are described in Section 5.6, infra.

Immunotherapies that promote immune response that can be employed in the methods described herein are described in Section 5.8, infra.

In some embodiments, the cancer to be treated is a blood cancer. The blood cancer can be a leukemia, a lymphoma, a myeloma, or a combination thereof. A blood cancer that can be treated in accordance with the methods described in this disclosure can be, but is not limited to: acute lymphoblastic leukemia, chronic lymphocytic leukemia, acute myelogenous leukemia, chronic myelogenous leukemia, hairy cell leukemia, T-cell prolymphocytic leukemia, Large granular lymphocytic leukemia, adult T-cell leukemia, plasma cell leukemia, Hodgkin lymphoma, Non-Hodgkin lymphoma, or multiple myeloma

In other embodiments, the cancer to be treated is a solid tumor cancer. The solid tumor cancer can be, but is not limited to, a sarcoma, a carcinoma, a lymphoma, a germ cell tumor, a blastoma, or a combination thereof. A solid tumor cancer that can be treated in accordance with the methods described in this disclosure can be, but is not limited to: breast cancer, lung cancer, ovary cancer, stomach cancer, pancreatic cancer, larynx cancer, esophageal cancer, testes cancer, liver cancer, parotid cancer, biliary tract cancer, colon cancer, rectum cancer, cervix cancer, uterus cancer, endometrium cancer, renal cancer, bladder cancer, prostate cancer, thyroid cancer, melanoma, or non-small cell lung cancer. In a specific embodiment, the cancer is lung cancer (e.g., non-small cell lung cancer), thyroid cancer, or melanoma.

In a specific embodiment, the patient's cancer is resistant to a therapy for the cancer previously administered to the patient. In some embodiments, the therapy for the cancer previously administered to the patient is chemotherapy. In other embodiments, the therapy for the cancer previously administered to the patient is radiation therapy.

In certain embodiments, the methods of treating a cancer as described above involve the killing or inhibition of proliferation of cancer cells, cancer stem cells, cancer progenitor cells, and/or cancer initiating cells, which do not have detectable MHC expression or have low levels of MHC expression (e.g., the cancer stem cells described in International Patent Application Publication No. WO 2011/038300 A1). In such embodiments, inhibition of the activity of a kinase that is ALK or ERBB2 upregulates class I MHC gene expression on cancer cells, cancer stem cells, cancer progenitor cells, and/or cancer initiating cells (in human patients, such inhibition upregulates HLA-A expression, and preferably also upregulates HLA-B expression and HLA-C expression on cancer cells, cancer stem cells, cancer progenitor cells, and/or cancer initiating cells).

5.2. Treatment of Infectious Disease

In another aspect, provided herein are methods of treating an infection in a patient comprising: (i) administering to the patient an inhibitor of the activity of a kinase, and (ii) administering to the patient an immunotherapy that promotes an immune response against the infection; wherein the kinase is ALK.

In another aspect, provided herein are methods of treating an infection in a patient comprising: (i) administering to the patient an inhibitor of the activity of a kinase, and (ii) administering to the patient an immunotherapy that promotes an immune response against the infection; wherein the kinase is ERBB2.

According to the invention, and without intending to be bound by a mechanism, inhibition of the activity of a kinase that is ALK or ERBB2 upregulates class I MHC gene expression on infected cells (in human patients, such inhibition upregulates HLA-A expression, and preferably also upregulates HLA-B expression and HLA-C expression on infected cells). In various embodiments, the inhibitor is administered in a subclinical amount. A subclinical amount of the inhibitor refers to an amount of the inhibitor at which no clinical effect or less than optimal clinical effect is detected when the inhibitor is administered alone (i.e., not in combination with the immunotherapy). In specific embodiments, the subclinical amount is lower than the amount of the inhibitor commonly used in the standard-of-care therapy for the infection to be treated. In specific embodiments wherein the inhibitor is FDA (Food and Drug Administration)-approved for treating the infection, the subclinical amount is lower than the FDA-approved amount for treating the infection.

In another aspect, provided herein are methods of generating a population of antigen-presenting cells for therapeutic administration to a patient having an infection, comprising culturing antigen-presenting cells that are loaded with or genetically engineered to express one or more immunogenic peptides or proteins derived from one or more antigens of the pathogen causing the infection in the presence of an inhibitor of the activity of a kinase, wherein the kinase is ALK. In another aspect, provided herein are methods of generating a population of antigen-presenting cells for therapeutic administration to a patient having an infection, comprising culturing antigen-presenting cells that are loaded with or genetically engineered to express one or more immunogenic peptides or proteins derived from one or more antigens of the pathogen causing the infection in the presence of an inhibitor of the activity of a kinase, wherein the kinase is ERBB2. In another aspect, provided herein are methods of treating an infection in a patient comprising generating a population of antigen-presenting cells according to methods described herein and administering to the patient the population of antigen-presenting cells. The antigen-presenting cells can be, for example, dendritic cells, cytokine-activated monocytes, or PBMCs. Preferably, the antigen-presenting cells are dendritic cells. Preferably, the antigen-presenting cells are autologous to the human patient (e.g., dendritic cells autologous to the human patient).

In another embodiment, provided herein are ex vivo methods of generating a population of antigen-specific T cells for therapeutic administration to a patient having an infection, comprising co-culturing T cells with antigen-presenting cells that are loaded with or genetically engineered to express one or more immunogenic peptides or proteins derived from one or more antigens of the pathogen causing the infection in the presence of an inhibitor of the activity of a kinase, wherein the kinase is ALK. In another embodiment, provided herein are ex vivo methods of generating a population of antigen-specific T cells for therapeutic administration to a patient having an infection, comprising co-culturing T cells with antigen-presenting cells that are loaded with or genetically engineered to express one or more immunogenic peptides or proteins derived from one or more antigens of the pathogen causing the infection in the presence of an inhibitor of the activity of a kinase, wherein the kinase is ERBB2. In another aspect, provided herein are ex vivo methods of treating an infection in a patient comprising generating a population of antigen-specific T cells according to methods described herein and administering to the patient the population of antigen-specific T cells. The antigen-presenting cells can be, for example, dendritic cells, cytokine-activated monocytes, or PBMCs. Preferably, the antigen-presenting cells are dendritic cells. Preferably, the antigen-presenting cells are autologous to the human patient (e.g., dendritic cells autologous to the human patient).

While the methods of treating an infection described in this disclosure are largely methods of combination therapy, the present invention also contemplates monotherapies using kinase inhibitors alone to treat infection. Therefore, in another aspect, provided herein are methods of treating an infection in a patient comprising administering to the patient an inhibitor of the activity of a kinase, wherein the kinase is ALK; and in another aspect, provided herein are methods of treating an infection in a patient comprising administering to the patient an inhibitor of the activity of a kinase, wherein the kinase is ERBB2.

Inhibitors of the activity of a kinase that is ALK and inhibitors of the activity of a kinase that is ERBB2, that can be employed in the methods described herein are described in Section 5.6, infra.

Immunotherapies that promote immune response that can be employed in the methods described herein are described in Section 5.8, infra.

In certain embodiments, the infection to be treated is an infection with a virus, bacterium, fungus, helminth or protist. In specific embodiments, the infection is an infection with a virus, such as herpesvirus, cytomegalovirus, Epstein Bar virus, polyoma virus, polyoma BK virus, John Cunningham virus, adenovirus, human immunodeficiency virus, influenza virus, ebola virus, poxvirus, norovirus, rotavirus, rhabdovirus, or paramyxovirus, etc. In a specific embodiment, the infection is an infection with herpesvirus. In another specific embodiment, the infection is an infection with cytomegalovirus. In another specific embodiment, the infection is an infection with Epstein Bar virus. In another specific embodiment, the infection is an infection with polyoma virus.

In a specific embodiment, the patient's infection is resistant to a therapy for the infection previously administered to the patient. In some embodiments, the therapy for the infection previously administered to the patient is antibiotics. In other embodiments, the therapy for the infection previously administered to the patient is anti-viral therapy.

5.3. Treatment of Autoimmune Disease

In another aspect, provided herein are methods of treating an autoimmune disease in a patient comprising: (i) administering to the patient an activator of the activity of a kinase, and optionally (ii) administering to the patient an immunosuppressive therapy that suppresses the immune response associated with the autoimmune disease; wherein the kinase is ALK.

In another aspect, provided herein are methods of treating an autoimmune disease in a patient comprising: (i) administering to the patient an activator of the activity of a kinase, and optionally (ii) administering to the patient an immunosuppressive therapy that suppresses the immune response associated with the autoimmune disease; wherein the kinase is ERBB2.

According to the invention, and without intending to be bound by a mechanism, activation of the activity of a kinase that is ALK or ERBB2 downregulates class I MHC gene expression on cells to which an autoimmune response is directed (in human patients, such activation downregulates HLA-A expression, and preferably also downregulates HLA-B expression and HLA-C expression on cells to which an autoimmune response is directed). In various embodiments, the activator is administered in a subclinical amount. A subclinical amount of the activator refers to an amount of the activator at which no clinical effect or less than optimal clinical effect is detected when the activator is administered alone (i.e., not in combination with the immunosuppressive therapy). In specific embodiments, the subclinical amount is lower than the amount of the activator commonly used in the standard-of-care therapy for the autoimmune disease to be treated. In specific embodiments wherein the activator is FDA (Food and Drug Administration)-approved for treating the autoimmune disease, the subclinical amount is lower than the FDA-approved amount for treating the autoimmune disease.

While the methods of treating an autoimmune disease described in this disclosure are largely methods of combination therapy, the present invention also contemplates monotherapies using kinase activators alone to treat autoimmune disease. Therefore, in another aspect, provided herein are methods of treating an autoimmune disease in a patient comprising administering to the patient an activator of the activity of a kinase, wherein the kinase is ALK; and in another aspect, provided herein are methods of treating an autoimmune disease in a patient comprising administering to the patient an activator of the activity of a kinase, wherein the kinase is ERBB2.

Activators of the activity of a kinase that is ALK and activators of the activity of a kinase that is ERBB2, that can be employed in the methods described herein are described in Section 5.7, infra.

Immunosuppressive therapies that suppress immune response that can be employed in the methods described herein are described in Section 5.9, infra.

An autoimmune disease that can be treated in accordance with the methods described in this disclosure can be, but is not limited to: Addison's disease, alopecia areata, ankylosing spondylitis, celiac sprue disease, Graves' disease, Hashimoto's thyroiditis, inflammatory bowel disease, lupus, multiple sclerosis, polymyalgia rheumatic, psoriasis, reactive arthritis, rheumatoid arthritis, scleroderma, Sjögren's syndrome, systemic lupus erythematosus, type 1 diabetes, temporal arteritis, vasculitis, or vitiligo. In a specific embodiment, the autoimmune disease is multiple sclerosis, type 1 diabetes, ankylosing spondylitis, or Hashimoto's thyroiditis.

In a specific embodiment, the patient's autoimmune disease is resistant to a therapy for the autoimmune disease previously administered to the patient. In some embodiments, the therapy for the autoimmune disease previously administered to the patient is an immunosuppressive therapy, such as those immunosuppressive therapies described in Section 5.9, supra.

5.4. Treatment of Graft-versus-Host Diseases

In another aspect, provided herein are methods of treating graft-versus-host disease (GvHD) in a patient comprising: (i) administering to the patient an activator of the activity of a kinase, and optionally (ii) administering to the patient an immunosuppressive therapy that suppresses the immune response associated with the GvHD; wherein the kinase is ALK.

In another aspect, provided herein are methods of treating graft-versus-host disease (GvHD) in a patient comprising: (i) administering to the patient an activator of the activity of a kinase, and optionally (ii) administering to the patient an immunosuppressive therapy that suppresses the immune response associated with the GvHD; wherein the kinase is ERBB2.

According to the invention, and without intending to be bound by a mechanism, activation of the activity of a kinase that is ALK or ERBB2 downregulates class I MHC gene expression on grafted cells (in human patients, such activation downregulates HLA-A expression, and preferably also downregulates HLA-B expression and HLA-C expression on grafted cells). In various embodiments, the activator is administered in a subclinical amount. A subclinical amount of the activator refers to an amount of the activator at which no clinical effect or less than optimal clinical effect is detected when the activator is administered alone (i.e., not in combination with the immunosuppressive therapy). In specific embodiments, the subclinical amount is lower than the amount of the activator commonly used in the standard-of-care therapy for the GvHD to be treated. In specific embodiments wherein the activator is FDA (Food and Drug Administration)-approved for treating the GvHD, the subclinical amount is lower than the FDA-approved amount for treating the GvHD.

While the methods of treating a GvHD described in this disclosure are largely methods of combination therapy, the present invention also contemplates monotherapies using kinase activators alone to treat GvHD. Therefore, in another aspect, provided herein are methods of treating a GvHD in a patient comprising administering to the patient an activator of the activity of a kinase, wherein the kinase is ALK; and in another aspect, provided herein are methods of treating a GvHD in a patient comprising administering to the patient an activator of the activity of a kinase, wherein the kinase is ERBB2.

Activators of the activity of a kinase that is ALK and activators of the activity of a kinase that is ERBB2, that can be employed in the methods described herein are described in Section 5.7, infra.

Immunosuppressive therapies that suppress immune response that can be employed in the methods described herein are described in Section 5.9, infra.

In some embodiments, the GvHD to be treated is an acute GvHD. In other embodiments, the GvHD to be treated is a chronic GvHD.

In a specific embodiment, the GvHD to be treated results from an allogeneic donor leukocyte infusion. In another specific embodiment, the GvHD to be treated results from an allogeneic hematopoietic stem cell transplantation (e.g., a bone marrow transplantation, a peripheral blood stem cell transplantation, or a cord blood transplantation). In another specific embodiment, the GvHD to be treated results from an allogeneic blood transfusion.

In a specific embodiment, the patient's GvHD is resistant to a therapy for the GvHD previously administered to the patient. In some embodiments, the therapy for the GvHD previously administered to the patient is an immunosuppressive therapy, such as those immunosuppressive therapies described in Section 5.9, supra.

5.5. Reduction of Risk of or Prevention of Solid Organ Transplant Rejection

In another aspect, provided herein are methods of reducing the risk of (e.g., prevention of) solid organ transplant rejection in a patient comprising: (i) administering to the patient an activator of the activity of a kinase, and optionally (ii) administering to the patient an immunosuppressive therapy that suppresses the immune response against the solid organ transplant; wherein the kinase is ALK.

In another aspect, provided herein are methods of reducing the risk of (e.g., prevention of) solid organ transplant rejection in a patient comprising: (i) administering to the patient an activator of the activity of a kinase, and optionally (ii) administering to the patient an immunosuppressive therapy that suppresses the immune response against the solid organ transplant; wherein the kinase is ERBB2.

According to the invention, and without intending to be bound by a mechanism, activation of the activity of a kinase that is ALK or ERBB2 downregulates class I MHC gene expression on solid organ transplant cells (in human patients, such activation downregulates HLA-A expression, and preferably also downregulates HLA-B expression and HLA-C expression on solid organ transplant cells). In various embodiments, the activator is administered in a subclinical amount. A subclinical amount of the activator refers to an amount of the activator at which no clinical effect or less than optimal clinical effect is detected when the activator is administered alone (i.e., not in combination with the immunosuppressive therapy). In specific embodiments, the subclinical amount is lower than the amount of the activator commonly used in the standard-of-care therapy for reducing the risk of (e.g., prevention of) solid organ transplant rejection. In specific embodiments wherein the activator is FDA (Food and Drug Administration)-approved for reducing the risk of (e.g., prevention of) solid organ transplant rejection, the subclinical amount is lower than the FDA-approved amount for reducing the risk of (e.g., prevention of) solid organ transplant rejection.

While the methods of reducing the risk of (e.g., prevention of) solid organ transplant rejection described in this disclosure are largely methods of combination therapy, the present invention also contemplates monotherapies using kinase activators alone for reducing the risk of (e.g., prevention of) solid organ transplant rejection. Therefore, in another aspect, provided herein are methods of reducing the risk of (e.g., prevention of) solid organ transplant rejection in a patient comprising administering to the patient an activator of the activity of a kinase, wherein the kinase is ALK; and in another aspect, provided herein are methods of reducing the risk of (e.g., prevention of) solid organ transplant rejection in a patient comprising administering to the patient an activator of the activity of a kinase, wherein the kinase is ERBB2.

Activators of the activity of a kinase selected from the group consisting of GRK7, EGFR, RET, and BRSK1, that can be employed in the methods described herein are described in Section 5.7, infra.

Immunosuppressive therapies that suppress immune response that can be employed in the methods described herein are described in Section 5.9, infra.

In specific embodiments, the solid organ transplant is a kidney transplant, a liver transplant, a heart transplant, an intestinal transplant, a pancreas transplant, a lung transplant, a small bowel transplant, a thymus transplant, or a combination thereof.

In a specific embodiment, the patient's solid organ transplant is resistant to a therapy for reducing the risk of (e.g., prevention of) solid organ transplant rejection previously administered to the patient. In some embodiments, the therapy for reducing the risk of (e.g., prevention of) solid organ transplant rejection previously administered to the patient is an immunosuppressive therapy, such as those immunosuppressive therapies described in Section 5.9, supra.

5.6. Inhibitors of ALK and Inhibitors of ERBB2

In some embodiments, the inhibitor of the activity of a kinase that is ALK or ERBB2 (as the case may be), is a small molecule inhibitor. In other embodiments, the inhibitor is an antibody or an antigen-binding fragment thereof that specifically binds to the kinase. In specific embodiments, the antibody or antigen-binding fragment thereof antagonizes the activity of the kinase. Antibodies or an antigen-binding fragments thereof that can be the inhibitor include, but are not limited to, monoclonal antibodies, polyclonal antibodies, multispecific antibodies (e.g., bispecific antibodies), and antibody fragments retaining antigen-binding activity, such as Fv, Fab, Fab′, F(ab′)₂, diabodies, linear antibodies, single-chain antibody molecules (e.g., single chain fragment variable fragment (scFv)), multispecific antibodies formed from antibody fragments. In a specific embodiment, the antibody is a monoclonal antibody, for example, a neutralizing monoclonal antibody. In other embodiments, the inhibitor is an oligonucleotide such as an aptamer, an shRNA, miRNA, siRNA, or antisense DNA.

In a specific embodiment, the kinase is ALK and the inhibitor is crizotinib, ceritinib, or alectinib.

In another specific embodiment, the kinase is ERBB2 and the inhibitor is trastuzumab or lapatinib. 5.7. Activators of ALK and Activators of ERBB2

In some embodiments, the activator of the activity of a kinase that is ALK or ERBB2 (as the case may be), is a soluble ligand (e.g., an activating protein ligand) of the kinase (e.g., where the kinase is a receptor), or a soluble ligand (e.g., an activating protein ligand) of a receptor that activates the kinase in vivo. In other embodiments, the activator is an antibody or an antigen-binding fragment thereof that specifically binds to the kinase. In specific embodiments, the antibody or antigen-binding fragment thereof agonizes the activity of the kinase. Antibodies or an antigen-binding fragments thereof that can be the activator include, but are not limited to, monoclonal antibodies, polyclonal antibodies, multispecific antibodies (e.g., bispecific antibodies), and antibody fragments retaining antigen-binding activity, such as Fv, Fab, Fab′, F(ab′)₂, diabodies, linear antibodies, single-chain antibody molecules (e.g., scFv), multispecific antibodies formed from antibody fragments. In a specific embodiment, the antibody is a monoclonal antibody.

5.8. Immunotherapies that Promote Immune Response

An immunotherapy promotes an immune response if it initiates an immune response or enhances a pre-existing immune response. In some embodiments of the methods of treating a cancer and the methods of treating an infection, which comprise administering to the patient an immunotherapy that promotes an immune response (in addition to administering to the patient an inhibitor of the activity of ALK or ERBB2), the immunotherapy initiates an immune response against the cancer or the infection (as the case may be). In other embodiments of the methods of treating a cancer and the methods of treating an infection, which comprise administering to the patient an immunotherapy that promotes an immune response (in addition to administering to the patient an inhibitor of the activity of ALK or ERBB2), the immunotherapy enhances a pre-existing immune response against the cancer or the infection (as the case may be)

In various embodiments, the immunotherapy can be a vaccine, an immune checkpoint blockade, an adoptive immunotherapy, a TCR (T-Cell Receptor) mimic antibody, a TCR based construct, an interferon (preferably interferon alpha or gamma), an anti-CD47 antibody, a SIRP alpha antagonist, an HDAC inhibitor, a cytokine, a TLR (Toll-Like Receptor) agonist, an epigenetic modulator that upregulates the expression of one or more MHCs (Major Histocompatibility Complexes) or upregulates antigen presentation, or a combination thereof.

In some embodiments, the immunotherapy is a vaccine. The vaccine can be any biological preparation that stimulates or elicits an endogenous immune response in the human patient against one or more antigens of the cancer or the pathogen causing the infection (as the case may be), such as, but are not limited to the ones described in Melief et al., 2015, J Clin Invest 125:3401-3412; Melero et al., 2014, Nat Rev Clin Oncol 11:509-524; and Guo et al., 2013, Adv Cancer Res 119:421-475; Nabel, 2013, N Engl J Med 368:551-560; and Saroja et al., 2011, Int J Pharm Investig 1: 64-74. In a specific embodiment, the vaccine comprises a peptide(s) or a protein(s) derived from the one or more antigens of the cancer or the pathogen causing the infection (as the case may be). In another specific embodiment, the vaccine comprises a nucleotide (e.g., a vector) expressing a peptide or a protein derived from the one or more antigens of the cancer or the pathogen causing the infection (as the case may be). In another specific embodiment, the vaccine is an antigen-presenting cell vaccine. In certain embodiments, the antigen-presenting cells in the antigen-presenting cell vaccine are loaded with one or more immunogenic peptides or proteins derived from one or more antigens of the cancer or the pathogen causing the infection (as the case may be). In other embodiments, the antigen-presenting cells in the antigen-presenting cell vaccine are genetically engineered to express one or more immunogenic peptides or proteins derived from one or more antigens of the cancer or the pathogen causing the infection (as the case may be). In preferred embodiments, the antigen-presenting cell vaccine is a dendritic cell vaccine.

In other embodiments, the immunotherapy is an immune checkpoint blockade. In specific embodiments, the immune checkpoint blockade is an antibody or an antigen-binding fragment thereof that specifically binds to and reduces the activity of an immune checkpoint protein. In a specific embodiment, the immune checkpoint blockade is an antibody or an antigen-binding fragment thereof that specifically binds to and blocks the activity of an immune checkpoint protein. Antibodies or an antigen-binding fragments thereof that can be the immune checkpoint blockade include, but are not limited to, monoclonal antibodies, polyclonal antibodies, multispecific antibodies (e.g., bispecific antibodies), and antibody fragments retaining antigen-binding activity, such as Fv, Fab, Fab′, F(ab′)₂, diabodies, linear antibodies, single-chain antibody molecules (e.g., scFv), multispecific antibodies formed from antibody fragments. In a specific embodiment, the antibody is a monoclonal antibody. In certain embodiments, the immune checkpoint blockade inhibits the activity of CTLA-4, PD-1, PD-L1, PD-L2, TIM-3, or LAG-3. In specific embodiments, the immune checkpoint blockade is an antibody or antigen-binding fragment thereof that specifically binds to and reduces the activity of CTLA-4, PD-1, PD-L1, PD-L2, TIM-3, or LAG-3. In a specific embodiment, the immune checkpoint blockade is tremelimumab. In another specific embodiment, the immune checkpoint blockade is nivolumab. In another specific embodiment, the immune checkpoint blockade is pembrolizumab. In another specific embodiment, the immune checkpoint blockade is ipilimumab.

In other embodiments, the immunotherapy is an adoptive immunotherapy, such as an adoptive T cell therapy. In specific embodiments, the adoptive T cell therapy involves the ex vivo stimulation, enrichment and/or expansion of non-genetically engineered antigen-specific T cells for infusion, for example as described in Yee, 2014, Immunol Rev 257:250-263; O'Reilly et al., 2011, Best Practice & Research Clinical Haematology 24:381-391; or O'Reilly et al., 2010, Semin Immunol 2010, 22:162-172. In other specific embodiments, the adoptive T cell therapy involves the infusion of genetically engineered T cells. In a specific embodiment, the adoptive T cell therapy is TCR-engineered T cells. A TCR-engineered T cell is a T cell that is genetically engineered to express on its surface a TCR that recognizes an antigen (which may be an intracellular antigen) of the cancer or the pathogen causing the infection (as the case may be). Preferably, a TCR expressed on the surface of a TCR-engineered T cell has high affinity for an antigen (which may be an intracellular antigen) of the cancer or the pathogen causing the infection (as the case may be). TCR-engineered T cells that can be employed in accordance with the present invention and technologies for generating TCR-engineered T cells are described in, for example, Stauss et al., 2015, Curr Opin Pharmacol 24:113-118; Sharpe and Mount, 2015, Dis Model Mech 8:337-350; Kunert et al., 2013, Front Immunol 4: 363; Stone et al., 2012, Methods Enzymol 503:189-222; and Park et al., 2011, Trends Biotechnol 29:550-557. In another specific embodiment, the adoptive T cell therapy is CAR T cells, wherein the antigen-binding domain of the CAR specifically binds to an antigen of the cancer. CARs are engineered receptors that provide both antigen binding and immune cell activation functions (Sadelain et al., 2013, Cancer Discovery 3:388-398). They usually comprise an antigen-binding domain (e.g., derived from a monoclonal antibody or the extracellular domain of a receptor), a transmembrane domain, an intracellular domain, and optionally a co-stimulatory domain. CARs can be used to graft the specificity of an antigen-binding domain onto an immune cell such as a T cell. CART cells are T cells that are genetically engineered to express CARs on their surface. CAR T cells that can be employed in accordance with the present invention and technologies for generating CAR T cells are described in, for example, Stauss et al., 2015, Curr Opin Pharmacol 24:113-118; Sharpe and Mount, 2015, Dis Model Mech 8:337-350; and Park et al., 2011, Trends Biotechnol 29:550-557.

In other embodiments, the immunotherapy is a TCR mimic antibody. TCR mimic antibodies are monoclonal antibodies that target against the WIC/antigen-peptide complexes presented on diseased cells (e.g., cancer cells or infected cells) (Dao et al., 2013, Oncolmmunology 2:e24678). They combine the recognition of antigen peptides (which may be peptides derived from intracellular antigens), analogous to that of a TCR, with the therapeutic potency and versatility of monoclonal antibodies. TCR mimic antibodies that can be employed in accordance with the present invention and technologies for generating TCR mimic antibodies, are described in, for example, Dubrovsky et al., 2015, Oncoimmunology 5:e1049803; Dao et al., 2013, Oncolmmunology 2:e24678; Cohen and Reiter, 2013, Antibodies, 2:517-534; and Dahan and Reiter, 2012, Expert Rev Mol Med 14:e6.

In other embodiments, the immunotherapy is a TCR based construct that encodes a soluble protein comprising the antigen recognition domain of a TCR. In certain embodiments, the immunotherapy is a soluble protein comprising the antigen recognition domain of a TCR. In preferred embodiments, the protein comprising the antigen recognition domain of a TCR comprises a second moiety for killing or inhibiting the proliferation of the cancer cells or infected cells (as the case may be) that are recognized by the TCR moiety. In a specific embodiment, the protein comprising the antigen recognition domain of a TCR is conjugated to a cytotoxic moiety. Such a cytotoxic moiety can be a cytotoxin, such as a radioisotope (e.g., a beta or alpha emitter), a cytotoxic drug (e.g., aureostatin), or a protein toxin (e.g., ricin). In another specific embodiment, the protein comprising the antigen recognition domain of a TCR also comprises an inflammatory cytokine, such as IL-2, TNF, or interferon gamma. In another specific embodiment, the protein comprising the antigen recognition domain of a TCR also comprises an antibody that specifically binds to a surface antigen on immune cells, such as T cells (e.g., an anti-CD3 antibody, such as an anti-CD3 scFv). In a further specific embodiment, the protein comprising the antigen recognition domain of a TCR is an immune mobilizing monoclonal TCR against cancer (ImmTAC). Soluble protein comprising the antigen recognition domain of a TCR and TCR based constructs that express such proteins, which can be employed in accordance with the present invention, and technologies for generating such soluble proteins and TCR based constructs are described in, for example, Oates et al., 2015, Mol Immunol 67:67-74; and Walseng et al., 2015, PLoS One 10:e0119559. The TCR based construct or the soluble protein comprising the antigen recognition domain of a TCR can be incorporated genetically or biochemically into a cell that affects the killing of the cancer, such as a T cell, a Natural Killer cell, or a monocyte.

In other embodiments, the immunotherapy is an interferon (preferably interferon alpha or gamma), an anti-CD47 antibody, a SIRP alpha antagonist, an HDAC inhibitor, a cytokine, a TLR agonist, or an epigenetic modulator that upregulates the expression of one or more MHCs (Major Histocompatibility Complexes) or upregulates antigen presentation. In a specific embodiment, the immunotherapy is an epigenetic modulator that upregulates the expression of one or more MHCs or upregulates antigen presentation that is a hypomethylating agent (e.g., azacytidine or decitabine). In another specific embodiment, the immunotherapy is an interferon that is interferon alpha or interferon gamma. In another specific embodiment, the immunotherapy is a cytokine that is IL2 (Interleukin-2), TNF (Tumor Necrosis Factor), interferon alpha or interferon gamma. In another specific embodiment, the immunotherapy is a TLR agonist that is a dsDNA (double-stranded DNA) TLR agonist. In another specific embodiment, the immunotherapy is a TLR agonist that is a dsRNA (double-stranded RNA) TLR agonist (e.g., polyinosinic-polycytidylic acid (poly(I:C)).

5.9. Immunosuppressive Therapies

An immunosuppressive therapy suppresses an immune response if it reduces or blocks an immune response. In some embodiments of the methods of treating an autoimmune disease, the methods of treating a GvHD, and the methods of reducing the risk of (e.g., prevention of) solid organ transplant rejection, which comprise administering to the patient an immunosuppressive therapy that suppresses an immune response (in addition to administering to the patient an activator of the activity of ALK or ERBB2), the immunosuppressive therapy reduces an immune response associated with the autoimmune disease or the GvHD or against the solid organ transplant (as the case may be). In other embodiments of the methods of treating an autoimmune disease, the methods of treating a GvHD, and the methods of reducing the risk of (e.g., prevention of) solid organ transplant rejection, which comprise administering to the patient an immunosuppressive therapy that suppresses an immune response (in addition to administering to the patient an activator of the activity of ALK or ERBB2), the immunosuppressive therapy blocks an immune response associated with the autoimmune disease or the GvHD or against the solid organ transplant (as the case may be).

The immunosuppressive therapy that can be employed in the methods of treating an autoimmune disease or a GvHD and the methods of reducing the risk of (e.g., prevention of) solid organ transplant rejection as described in this disclosure can be, but is not limited to, a glucocorticoid, a cytostatic (e.g., an alkylating agent, such as coclophosphamide, nitrosoureas, or platinum compound; or an antimetabolite, such as folic acid, purine analogue, pyrimidine analogue, protein synthesis inhibitor, methotrexate, azathioprine, mercaptopurine, fluorouracil, or a cytotoxic antibiotic), an antibody that can antagonize the activity of immune cells or cytokines (e.g., anti-CD20 antibody, anti-CD3 antibody, anti-IL2R antibody), a drug acting on immunophilins (e.g., ciclosporin, tacrolimus, or sirolimus), interferon beta, an opiod, a TNF antagonist (e.g., etanercept, infliximab, or adalimumab), mycophenolic acid, mycophenolate, fingolimod, or myriocin.

In various embodiments, the immunosuppressive therapy can be sirolimus, everolimus, rapamycin, one or more steroids, cyclosporine, cyclophosphamide, azathioprine, mercaptopurine, fluorouracil, fludarabine, interferon beta, a TNF decoy receptor, a TNF antibody, methotrexate, a T-cell antibody, an anti-CD20 antibody, a complement inhibitor, an anti-IL6 (Interleukin-6) antibody, an anti-IL2R (Interleukin-2 Receptor) antibody, anti-thymocyte globulin, fingolimod, mycophenolate, or a combination thereof.

In some embodiments, the immunosuppressive therapy is a TNF decoy receptor (e.g., etanercept). In other embodiments, the immunosuppressive therapy is a TNF antibody (e.g., infliximab). In other embodiments, the immunosuppressive therapy is a T-cell antibody (e.g., an anti-CD3 antibody, such as OKT3). In other embodiments, the immunosuppressive therapy is an anti-CD20 antibody (e.g., rituximab). In other embodiments, the immunosuppressive therapy is a complement inhibitor (e.g., eculizumab). In other embodiments, the immunosuppressive therapy is an anti-IL2R antibody (e.g., daclizumab).

5.10. Routes of Administration and Dosage

The inhibitors of kinases and activators of kinases as described above may be administered to patients by a variety of routes. These include, but are not limited to, parenteral, intranasal, intratracheal, oral, intradermal, topical, intramuscular, intraperitoneal, transdermal, intravenous, intratumoral, conjunctival, subcutaneous, and pulmonary routes.

The amount of an inhibitor of kinase or an activator of a kinase described herein or a pharmaceutical composition thereof to be administered to the patient will depend on the nature of the disease and the condition of the patient, and can be determined by standard clinical techniques and the knowledge of the physician.

The precise dose and regime to be employed in a composition will also depend on the route of administration, and the seriousness of the disease, and should be decided according to the judgment of the physician and each patient's circumstance.

In embodiments of combination therapies, the inhibitor of a kinase or the activator of a kinase (as the case may be) is administered concurrently or sequentially with the administration of the immunotherapy that promotes an immune response or the immunosuppressive therapy that suppresses an immune response (as the case may be), for example, at about the same time, the same day, or same week, or same period (treatment cycle) during which the immunotherapy that promotes an immune response or the immunosuppressive therapy that suppresses an immune response is administered, or on similar dosing schedules, or on different but overlapping dosing schedules. Preferably, the inhibitor of a kinase or the activator of a kinase (as the case may be) is administered concurrently with or shortly before (e.g., about 1, 2, 3, 4, 6, 8, 10, 12, 16, 20, or 24 hours before, or about 1, 2, 3, 4, 5, 6, or 7 days before) the administration of the immunotherapy that promotes an immune response or the immunosuppressive therapy that suppresses an immune response (as the case may be), as described above. The inhibitor of a kinase or the activator of a kinase (as the case may be), and the immunotherapy that promotes an immune response or the immunosuppressive therapy that suppresses an immune response (as the case may be) can be in the same pharmaceutical formulation or in separate formulations.

In a specific embodiment of the methods of treating cancer, the inhibitor of a kinase described in Sections 5.6, supra, is coupled with (e.g., conjugated to) an antibody that specifically binds to a cell surface marker uniquely expressed or expressed at higher levels (relative to non-cancerous cells) on the cancer cells, so that the inhibitor of a kinase is delivered specifically to the cancer cells. In another specific embodiment of the methods of treating cancer, the inhibitor of a kinase described in Section 5.6, supra, is coupled with (e.g., conjugated to) an antibody that specifically binds to a cell surface marker uniquely expressed or expressed at higher levels (relative to cells that are not cancer stem cells, cancer progenitor cells, and/or cancer initiating cells) on cancer stem cells, cancer progenitor cells, and/or cancer initiating cells of the cancer, so that the inhibitor of a kinase is delivered specifically to the cancer stem cells, cancer progenitor cells, and/or cancer initiating cells of the cancer.

In a specific embodiment of the methods of treating an infection, the inhibitor of a kinase described in Section 5.6, supra, is coupled with (e.g., conjugated to) an antibody that specifically binds to a cell surface marker uniquely expressed or expressed at higher levels (relative to uninfected cells) on the infected cells, so that the inhibitor of a kinase is delivered specifically to the infected cells.

In a specific embodiment of the methods of treating an autoimmune disease, the activator of a kinase described in Section 5.7, supra, is coupled with (e.g., conjugated to) an antibody that specifically binds to a cell surface marker uniquely expressed or expressed at higher levels (relative to wild-type cells) on cells to which an autoimmune response is derected, so that the activator of a kinase is delivered specifically to the cells that are the target of an autoimmune response.

In a specific embodiment of the methods of treating GvHD, the activator of a kinase described in Section 5.7, supra, is coupled with (e.g., conjugated to) an antibody that specifically binds to a cell surface marker uniquely expressed or expressed at higher levels (relative to non-grafted cells) on grafted cells, so that the activator of a kinase is delivered specifically to the grafted cells.

In a specific embodiment of the methods of reducing the risk of solid organ transplant rejection, the activator of a kinase described in Section 5.7, supra, is coupled with (e.g., conjugated to) an antibody that specifically binds to a cell surface marker uniquely expressed or expressed at higher levels (relative to cells not of the transplant) on the solid organ transplant, so that the activator of a kinase is delivered specifically to the solid organ transplant.

5.11. Patients

The patient referred to in this disclosure, can be, but is not limited to, a human or non-human vertebrate such as a wild, domestic or farm animal. In certain embodiments, the patient is a mammal, e.g., a human, a cow, a dog, a cat, a goat, a horse, a sheep, a pig, a rabbit, a rat, or a mouse. In a preferred embodiment, the patient is a human patient.

In a specific embodiment, the human patient is an adult (at least age 16). In another specific embodiment, the human patient is an adolescent (age 12-15). In another specific embodiment, the patient is a child (under age 12).

5.12. Methods of Treating a Patient Who has Failed a Previous Immunotherapy

The present invention also provides a method of treating a cancer or an infection in a patient who has failed a first immunotherapy for treatment of the cancer or infection (i.e., which immunotherapy is intended to promote an immune response against the cancer or infected cells), comprising: (i) administering to the patient an inhibitor of the activity of a kinase, and (ii) subsequently or concurrently administering to the patient a second immunotherapy for treatment of the cancer or infection (i.e., which immunotherapy is intended to promote an immune response. In a specific embodiment, the second immunotherapy targets different antigens associated with the cancer or infection than the first immunotherapy. In another specific embodiment, the second immunotherapy targets the same antigens associated with the cancer or infection as the first immunotherapy. In another specific embodiment, the second immunotherapy and the first immunotherapy are the same. While not intending to be bound by mechanism, as shown in Example 1 below, treatment with an inhibitor of a kinase that negatively regulates MHC Class I expression such as ALK or RET changed the antigen peptide repertoire of cancer cells.

In a specific embodiment, the kinase is ALK. In another specific embodiment, the kinase is ERBB2. In another specific embodiment, the kinase is a kinase that negatively regulates MHC Class I expression as described in International Patent Application No. PCT/US2017/022099 (International Patent Application Publication No. WO 2017/160717) (e.g., GRK (G protein-coupled receptor kinase 7), MAP2K1 (mitogen-activated protein kinase kinase 1), EGFR (epidermal growth factor receptor), RET (ret proto-oncogen), or BRSK1 (BR serine/threonine kinase 1)), which is incorporated by reference herein in its entirety.

### 6. EXAMPLES

The following non-limiting examples report the discovery of a set of kinases, including ALK and ERBB2, that are negative regulators of class I MHC gene expression. In addition, the examples demonstrate that inhibitors of certain kinases that negatively regulates class I MHC gene expression can alter the antigen peptide repertoire presented by HLA molecules.

### 6.1. Example 1: Regulation of Human Leukocyte Antigen (HLA) Class I Surface Expression Through the Inhibition of ALK and RET

6.1.1. Summary

HLA class I is a glycoprotein that binds to peptides of intracellular origin and displays them on the cell surface to be surveyed by T cells. Anaplastic lymphoma kinase (ALK) and ret proto-oncogene (RET) are both receptor tyrosine kinase (RTK) that are mutated in certain cancers and minimally expressed in other tissues. Using small molecule inhibitors against these RTKs, this Example showed that increasing concentrations of drug lead to a dose related increase in surface HLA. Corresponding increases in transcript and protein levels of HLA and antigen processing machinery were assayed through qPCR and western blots, respectively. Upregulation was seen in vivo as well. Killing assays in vitro showed increase tumor lysis with RET inhibition. Mass spectrometry of the eluted presented peptides showed that RET inhibition also lead to a change in the surface HLA-peptide repertoire. Hence, this Example illustrated that pharmacological inhibitors of ALK and RET could be a useful adjuvant for T cell based therapies and that pharmacological inhibitors of certain kinases may allow for targeting of novel epitopes that arise.

6.1.2. Materials and Methods

6.1.2.1. Cells Lines, Inhibitors, and Antibodies

The Karpas 299 and SUDHL-1 cells were obtained from the Dr. Anas Younes laboratory in Memorial Sloan Kettering Cancer Center (MSKCC). They were maintained in RPMI-1640 with 10% FBS and 2 mM L-glutamine. The TPC1 cell line was obtained from the Dr. James Fagin laboratory in MSKCC and maintained in DME media with 5% FBS and 2 mM L-glutamine. SKOV3 and U266 were purchased from the American Type Culture Collection (ATCC) and cultured in RPMI-1640 with 10% FBS and 2 mM L-glutamine. ALK inhibitors crizotinib, ceritinib and alectinib were purchased from Selleck Chemicals. RET inhibitor, AST 487, was purchased from MedChemExpress. Cabozantinib was purchased from Selleck Chemicals. Lapatinib and vemurafenib were purchased from Selleck Chemicals. Trastuzumab was from Genentech. Western antibodies for phospho-ERK (catalog 4370S), ERK (catalog 4696S), beta-2-microglobulin (catalog 12851S), and GAPDH (catalog 3683S) were purchased from Cell Signaling. HLA-A western antibodies (catalog sc-23446) were from Santa Cruz. The secondary antibodies, goat anti-mouse IgG-horseradish peroxidase (HRP), mouse anti-rabbit IgG-HRP, and donkey anti-goat IgG-HRP were purchased from Santa Cruz. Flow cytometry antibodies HLA-A02 (BB7.2) and HLA-A,B,C (W6/32) were purchased from eBioscience. PD-L1 (MIH1) antibody for flow was purchased from eBioscience.

6.1.2.2. Flow Cytometry

5×10⁴ cells were seeded in a 12 well plate and treated with drug for 72 hrs. If adherent cells, they were seeded one day before treatment. At 72 hrs, cells were harvested, washed and incubated on ice with appropriate fluorophore conjugated antibodies for 1 hr. Cells were then washed and incubated with a viability dye (propidium iodide at 1 μg/mL) and flow cytometry was run.

6.1.2.3. Western Blots

Cells were seeded in a 60 mm dish or 6 well plates. After the appropriate time point, cells were harvested and lysed and protein was quantified by a Lowry assay (Bio-Rad DC Protein Assay; #5000116). Protein levels were normalized and run on SDS PAGE gels (Bio-Rad). Protein was transferred to a nitrocellulose membrane using semi-dry transfer. Membrane was blocked and incubated with respective antibodies. When needed, secondary antibodies with HRP were incubated for 1 hr. Enhanced chemiluminescent substrate for HRP enzymes was used to image protein levels (Thermo-Fischer; #34095).

6.1.2.4. Real Time PCR

Cells were treated and incubated with appropriate small molecule inhibitors for 48 hrs and RNA was extracted using Qiagen RNA Easy Plus (Qiagen; #74134). Afterwards, cDNA was created using qScript cDNA SuperMix (Quantabio; #95048). qPCR was performed using PerfeCTa FastMix II (Quantabio; #95118) and TaqMan real time probes were purchased from Life Technologies: HLA-A (Hs01058806_g1), beta-2 microgobulin (Hs00187842_m1), TAP1 (Hs00388677_m1), TAP2 (Hs00241060_m1), and TBP (Hs00427620_m1).

6.1.2.5. ADCC

Peripheral blood mononuclear cells were derived from healthy donors by Ficoll density centrifugation after receiving informed consent on Memorial Sloan Kettering Institutional Review board-approved protocols. TPC1 cells treated with RET inhibitor drugs and DMSO control were labeled with chromium-51 and co-cultured with PBMCs and ESK (or its isotype control, hIgG1). Different E:T ratios were used and after 5 hrs of incubation at 37° C., the supernatant was harvested and chromium levels were measured by Chromium-51 release assay (Perkin Elmer). Higher chromium levels indicated higher levels of cytotoxicity.

6.1.2.6. Animal Studies

Female NSG (NOD.Cg-Prkdc^{scid}Il2rg^{tm1Wj1}/SzJ) and NRG (NOD.Cg-Rag1^{tm1Mom}Il2rg^{tm1Wj1}/SzJ) mice were purchased from the Jackson Laboratory or MSKCC breeding facility at 5-10 weeks old. For RET and ALK experiments, 2.5-6×10⁶ tumor cells were subcutaneously injected into the flank of mice and when tumors were palpable, mice were treated daily with drug or vehicles through oral gavage. At day 7, tumors were harvested and flow cytometry was run to determine effect of inhibitors on HLA and PD-L1 on the tumor cells.

6.1.2.7. Mass Spectrometry

TPC1 cells were treated with DMSO, 10 nM AST487, or 100 nM cabozantinib. Karpas 299 cells were treated with DMSO, 100 nM crizotinib or 100 nM ceritinib. After 72 hrs, cells were harvested, washed, lysed. The lysate was run through an activated Sepharose-CNBr column coupled to the W6/32 antibody to bind HLA-A,B,C from the cell. The peptides bound by those HLAs and captured on the column were then eluted and mass spectrometry was run on those peptides.

6.1.3. Results

6.1.3.1. ALK Inhibition Increases Cell Surface HLA and Overall HLA Protein Levels Through MAPK

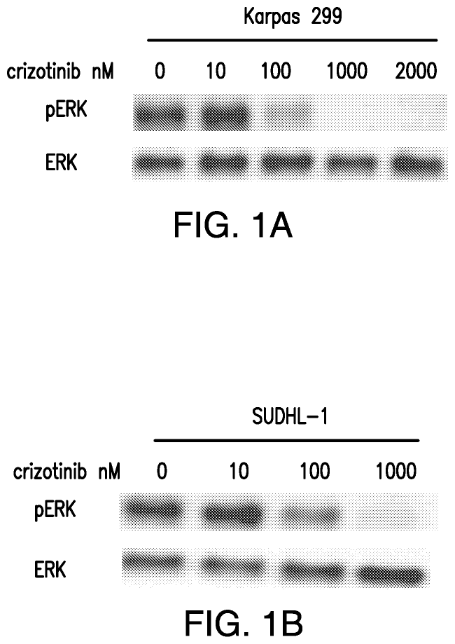

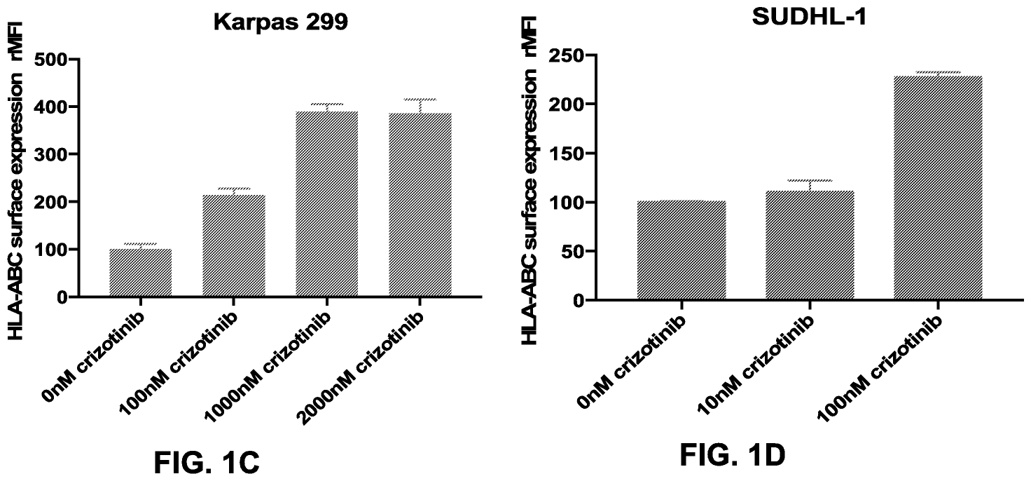

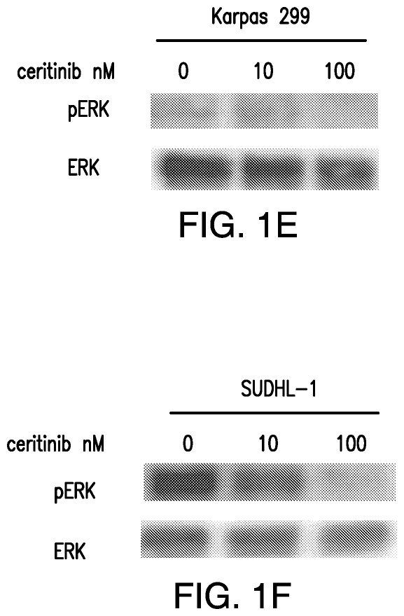

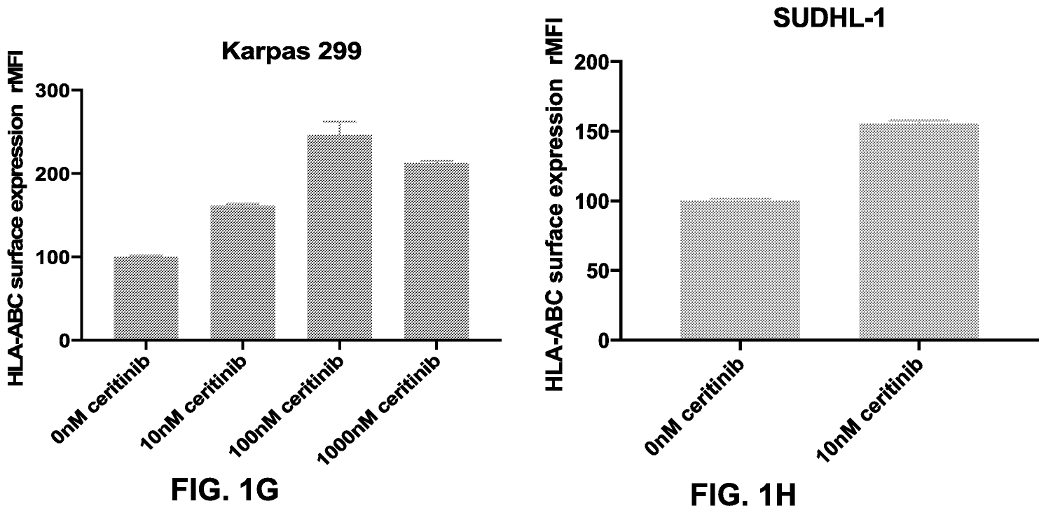

Crizotinib is a small molecule tyrosine kinase inhibitor that is FDA approved for the treatment of ALK positive non-small cell lung cancer (NSCLC). Increasing concentrations of crizotinib on Karpas 299, a NPM (nucleophosmin-anaplastic lymphoma kinase)-ALK+anaplastic large-cell lymphoma (ALCL) cell line, showed a dose-related reduction of pERK at 3 hours, indicating that inhibition of ALK shuts down the MAPK pathway (FIG. 1A). Flow cytometric analysis of HLA levels after a 72 hour incubation with crizotinib with Karpas 299 showed an inverse dose response with decreasing levels of pERK leading to increased levels of surface HLA-A,B,C (FIG. 1C). HLA levels on Karpas 299 cells treated with 1 uM crizotinib increased 4-fold compared to control cells treated with DMSO. A plateau in surface HLA upregulation was seen at higher concentrations of crizotinib due to the complete shut down of ERK phosphorylation at lower doses. Similar results were seen with SUDHL-1, another NPM-ALK+ALCL line, in which crizotinib also inhibited pERK expression and led to increased surface HLA in a dose-related manner (FIGS. 1B and 1D).

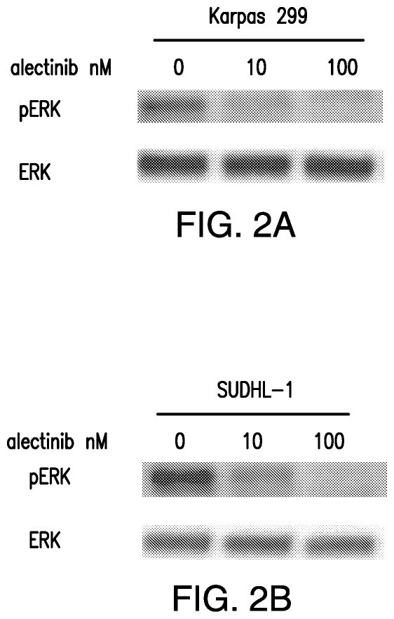

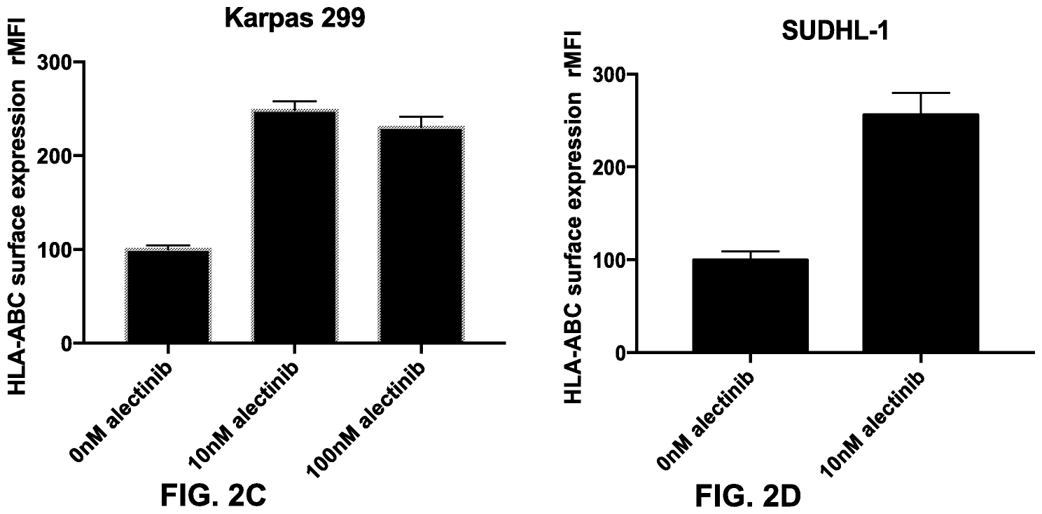

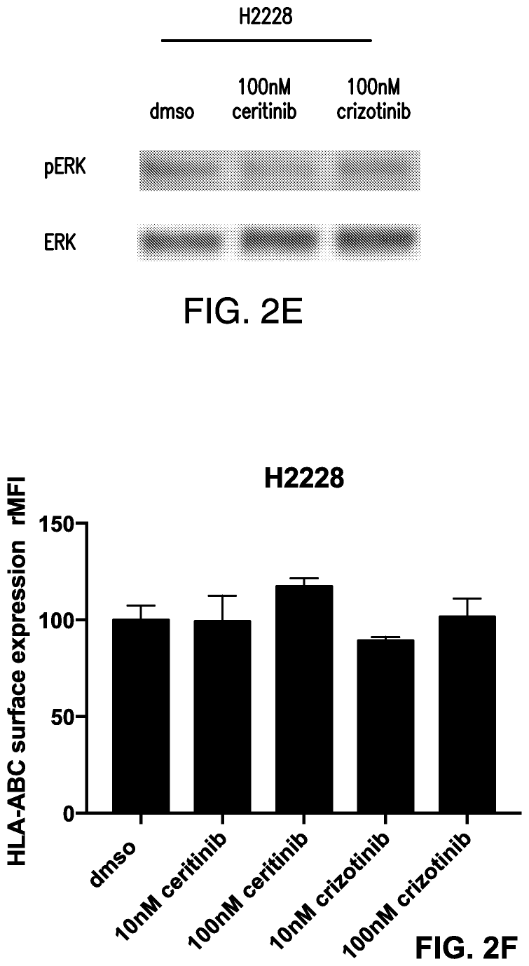

To confirm that ALK inhibition was the mechanistic target for HLA regulation, another small molecule ALK inhibitor, ceritinib (LDK378), a second-generation FDA-approved ALK inhibitor used to treat non-small cell lung cancers, was also tested. Ceritinib inhibits resistance mutations arising from crizotinib treatment and is more potent than crizotinib (Sullivan and Planchard, 2016, Ther Adv Med Oncol 8(1):32-47). Treatment of Karpas 299 and SUDHL-1 with increasing concentrations of ceritinib also shut down pERK levels (FIGS. 1E and 1F). Cells were comparatively more sensitive to ceritinib than crizotinib, and died at lower concentrations. However, cell surface HLA increased in both cell lines in a dose-dependent manner (FIGS. 1G and 1H). Similar results were seen with alectinib, another second-generation ALK inhibitor (FIG. 2A-2D). As these 3 drugs exhibit different classes of off targets, similar results provide strong confidence that the increase in HLA seen was a result of ALK inhibition. The relationship between MAPK inhibition and HLA upregulation was also seen with the EML4 (echinoderm microtubule associated protein-like 4)-ALK fusion cell line H2228. 100 nM of crizotinib did not change pERK levels and consequenctly surface HLA levels did not change. 100 nM of ceritinib slightly decreased pERK levels and a corresponding slight increase in surface HLA levels was seen (FIGS. 2E and 2F). Therefore in multiple cell lines, using several inhibitors of ALK, the inhibition of ERK output by the drug correlated with cell surface HLA levels.

6.1.3.2. RET Inhibition Also Increases Cell Surface HLA Through Inhibition of the MAPK Pathway

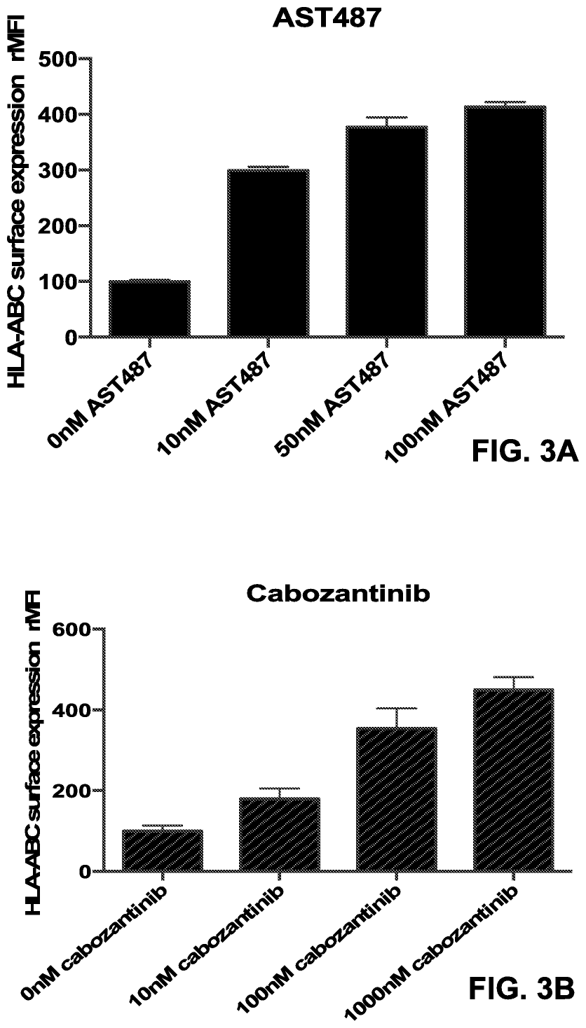

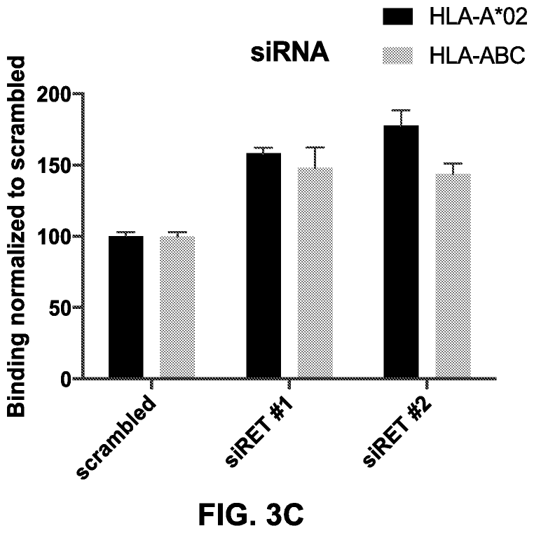

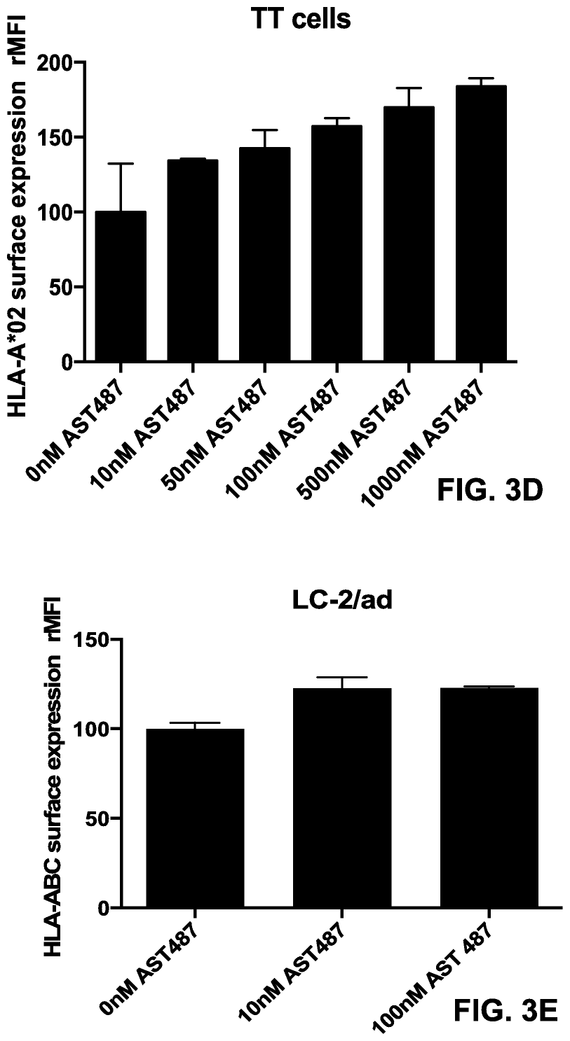

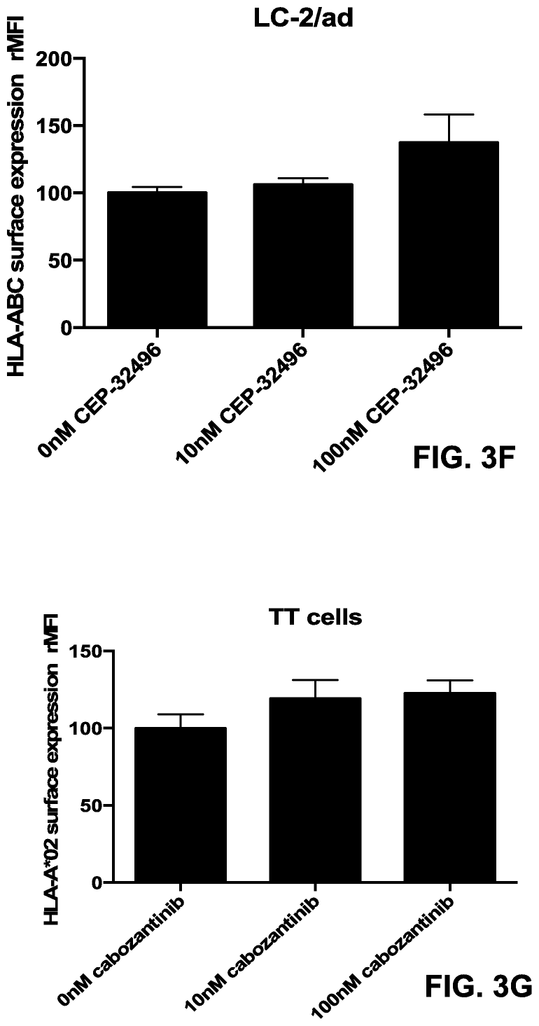

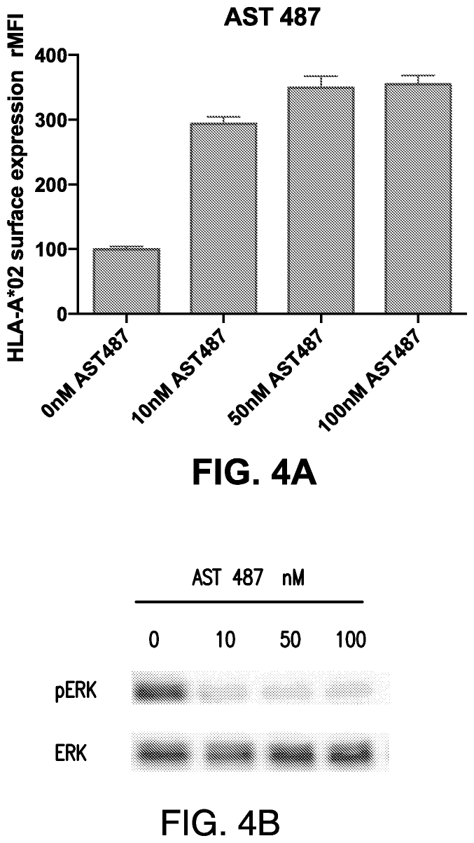

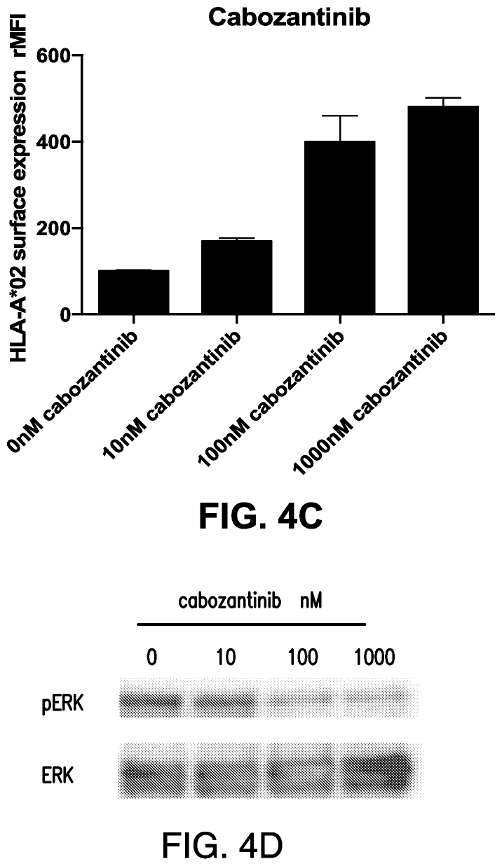

RET is found mutated in thyroid cancers and a small percentage of NSCLC. AST487 is a RET tyrosine kinase inhibitor that has been shown to inhibit growth of thyroid cell lines with activating RET mutations. TPC1 is a papillary thyroid cancer cell line that has a CCDCl6 (coiled-coil domain containing 6)-RET fusion protein driving constitutive activation of RET. Treatment of TPC1 cells with AST487 led to a 3- to 4-fold increase in surface HLA-A,B,C levels at 72 hours (FIG. 3A). In addition to pan-HLA increases, the HLA-A*02:01 molecules on TPC1 were also increased with AST487 drug treatment, indicating that the individual alleles are also increased (FIG. 4A). Inhibition of pERK was seen at low concentrations of 10 nM AST487 (FIG. 4B).

Cabozantinib, a small molecule inhibitor of RET, MET, and VEGF2 that is FDA approved for treatment of medullary thyroid cancer, showed similar regulation of HLA. Cabozantinib was incubated for 72 hours with TPC1 and cell surface HLA-A,B,C and HLA-A*02:01 were measured. At 100 nM, there wasabout a 4-fold increase in surface HLA (FIG. 4C; FIG. 3B). A dose response relationship was seen with increasing concentrations of drug. Western blot analysis confirmed decreasing pERK levels with inhibitor treatment (FIG. 4D). By use of these different RET inhibitors, which have varying off targets, it was confirmed that inhibition of RET (and not another off target) is causing the increase in HLA. To further validate that RET inhibition caused the increases in HLA, siRNAs were also applied against RET. Knockdown of RET by siRNA showed similar increases in HLA (FIG. 3C).

To determine if these findings occurred with other RET mutations in other cancers, a lung cancer cell line LC-2/ad, which has the same CCDCl6-RET fusion as TPC1 (Matsubara), was tested. TT cells, which are a medullary thyroid cell line that harbors a MEN2A mutation (cysteine to tryptophan mutation at codon 634) leading to dimerization ad activation, were also examined. Though HLA levels of these lines did not increase as much with RET inhibition, cell surface HLA was still upregulated, supporting the inhibition of RET for regulating HLA (FIGS. 3D-3G). Due to more robust upregulation in TPC1, these cells were used for other RET inhibition studies.

6.1.3.3. Increased Cytotoxicity Following MAPK Pathway Inhibition

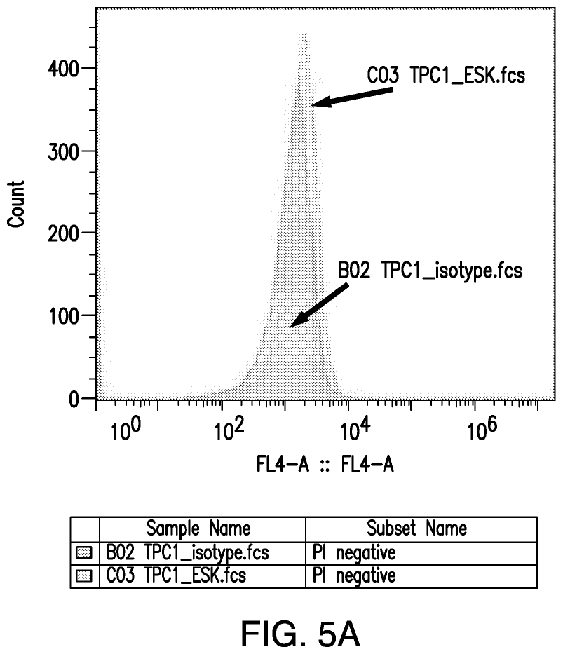

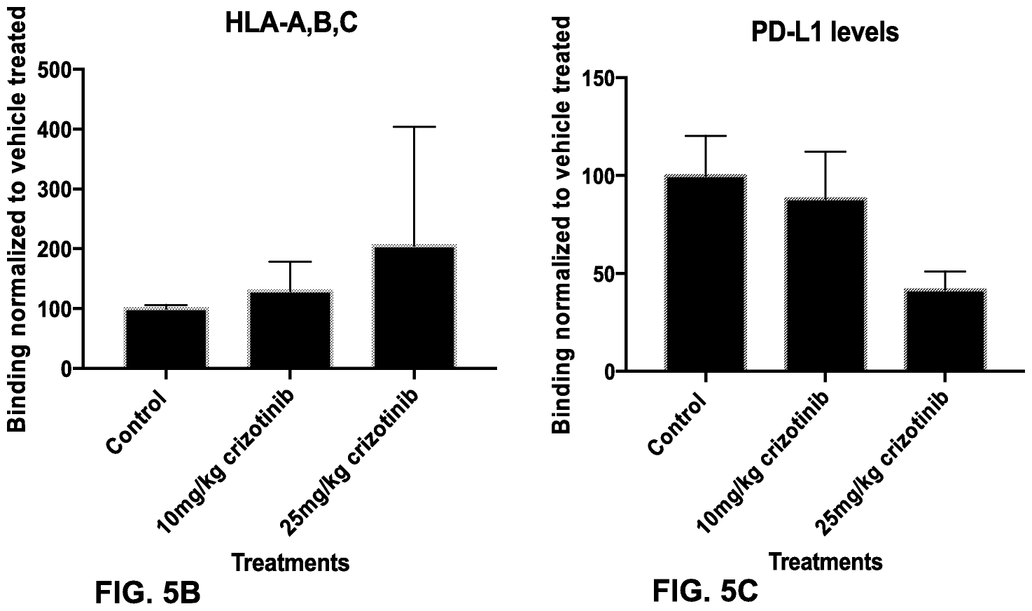

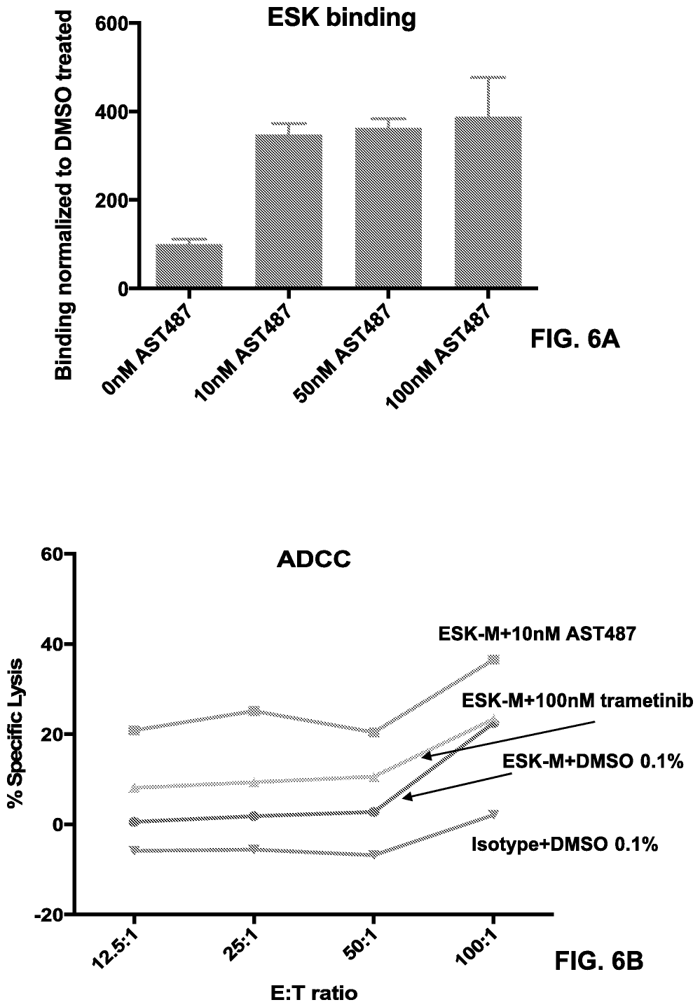

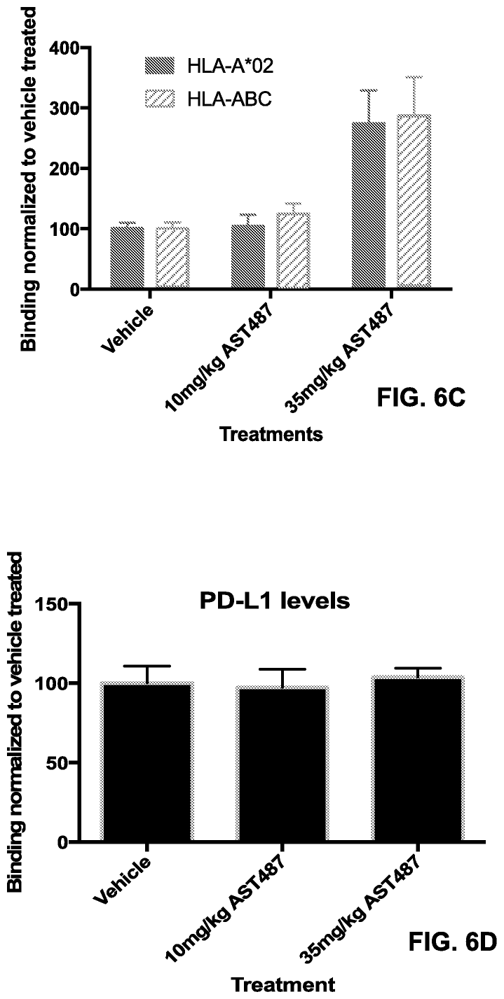

TCR mimic monoclonal antibodies (TCRm) recognize the peptide/MHC complex epitopes similar to that of a TCR or T cell, but have the advantageous pharmacological properties of an antibody, such as long half-life, therapeutic potency, and versatility (Dubrovsky et al., 2014, Blood 123(21):3296-3304). The TCRm, ESK1 (reactive with a peptide from the oncofetal antigen WT1), was used as a tool to ask if there was immune effector functional utility of the increased HLA expression following MAPK inhibition. ESK1 binds to the RMFPNAPYL (SEQ ID NO:1) peptide of Wilms' tumor gene 1 (WT1) in complex with HLA-A*02:01 (Veomett et al., 2014, Clin Cancer Res 20(15):4036-4046) and also to a non-WT1-derived peptide expressed on TPC1 cells (Gejman et al., 2018, Prospective identification of cross-reactive human peptide-MHC ligands for T cell receptor based therapies. Manuscript submitted for publication). TPC1 cells were bound to ESK1 though they do not express WT1 (FIG. 5A). Flow cytometry showed increased ESK binding following RET inhibition (FIG. 5A). Increased antibody-dependent cell cytotoxicity (ADCC) activity was observed when TPC1 cells were pre-incubated with the RET inhibitor. Cells were treated with DMSO, 10 nM AST487, or 100 nM trametinib (a MEK inhibitor) for 72 hrs before labeling with Chromium-51. After the addition of ESK and peripheral blood mononuclear cells for 5 hrs, chromium release was measured. At a 50:1 E:T (effector to target ratio), cells pretreated with AST487 showed 20% specific lysis, compared to the 3% specific lysis of the DMSO treated group. The 10% in the trametinib treated group improved lysis by ESK1 by about 10% at 3 or 4 E:T ratios (FIG. 6B).

6.1.3.4. Increase of HLA Expression In Vivo after MAPK Inhibition

To determine if the inhibitors produced similar therapeutic improvements in vivo, mice bearing the TPC1 tumor were treated with RET inhibitors. The highly immunodeficient NRG (NOD-Rag1null IL2rgnull) mice were subcutaneously injected with 2.5×10⁶ luciferase tagged TPC1 cells in their right flank. When the tumors were palpable, mice were given vehicle, 10 mg/kg AST487, or 35 mg/kg AST 487 through once daily oral gavage for 7 days. Afterwards, cells were immediately harvested and stained with antibodies against HLA-A*02:01 and HLA-ABC. Dose related increase in HLA levels were seen with AST487, indicating that HLA also can be regulated in vivo with RET inhibition (FIG. 6C). Moreover, PD-L1 levels were measured and no increase in PD-L1 was seen (FIG. 6D). These data suggest a potential for RET inhibition in combination with T cell based therapies. NRG mice were also injected with Karpas 299 cells and treated with vehicle, 10 mg/kg, or 25 mg/kg crizotinib. Levels of HLA increased slightly in a dose-response manner, though not all mice responded (FIG. 5B). Interestingly, the mouse with the largest tumor shrinkage was also the one that had the largest increase in surface HLA. PD-L1 levels decreased with increasing doses of crizotinib (FIG. 5C).

6.1.3.5. ALK and RET Inhibition Increase Surface HLA Through Transcript and Protein Levels

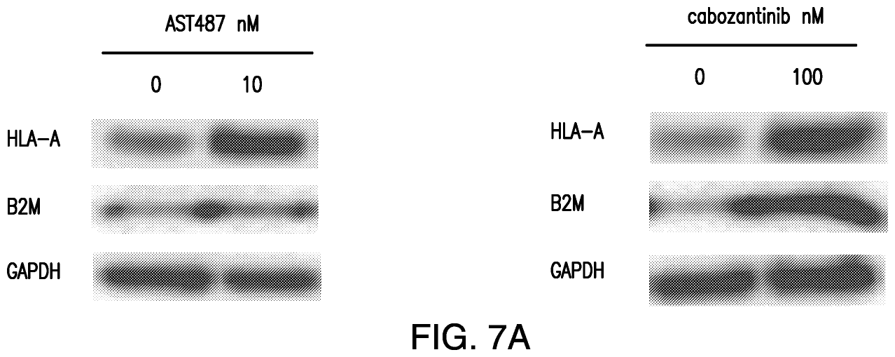

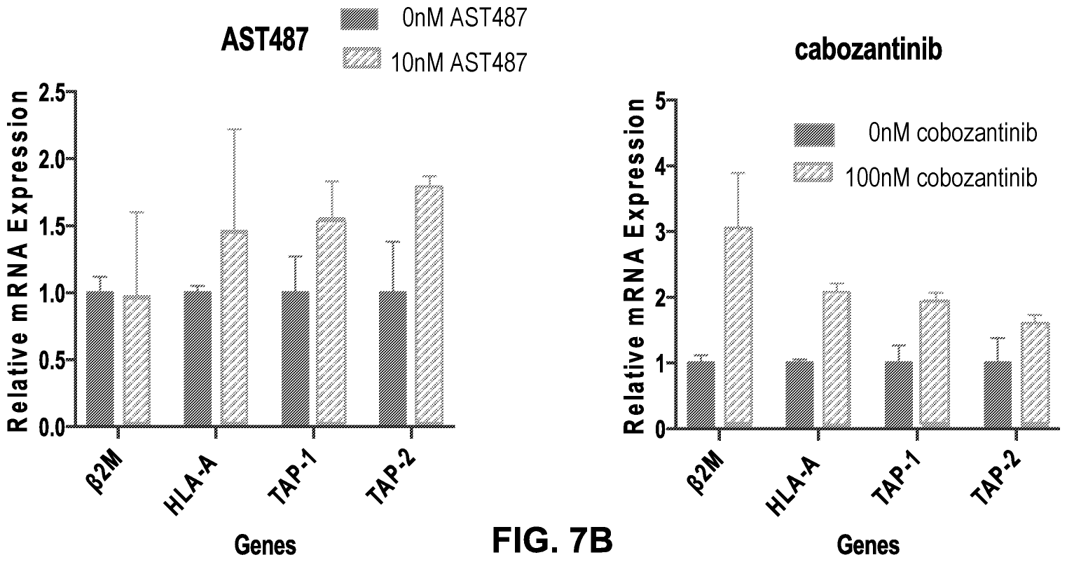

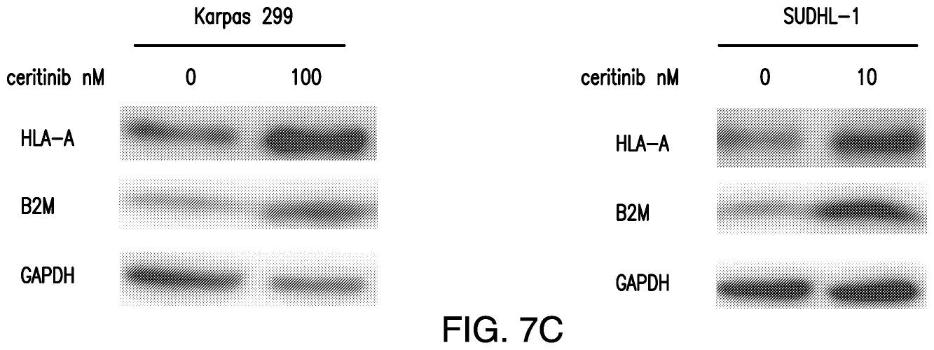

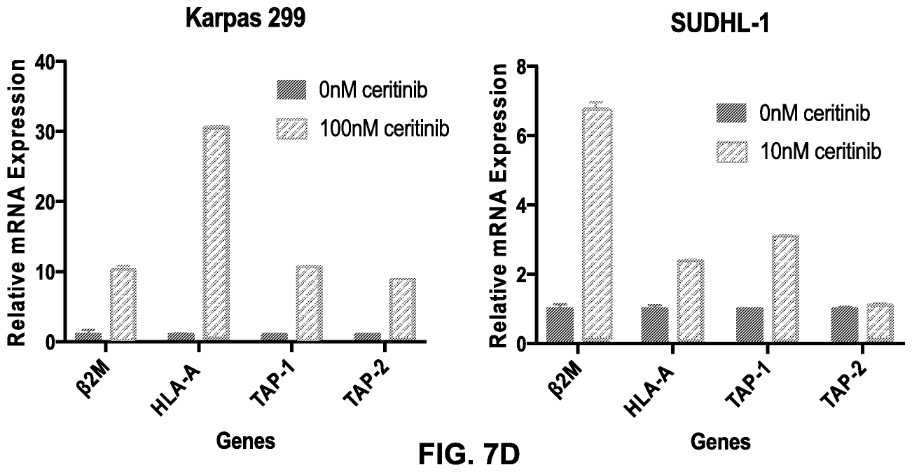

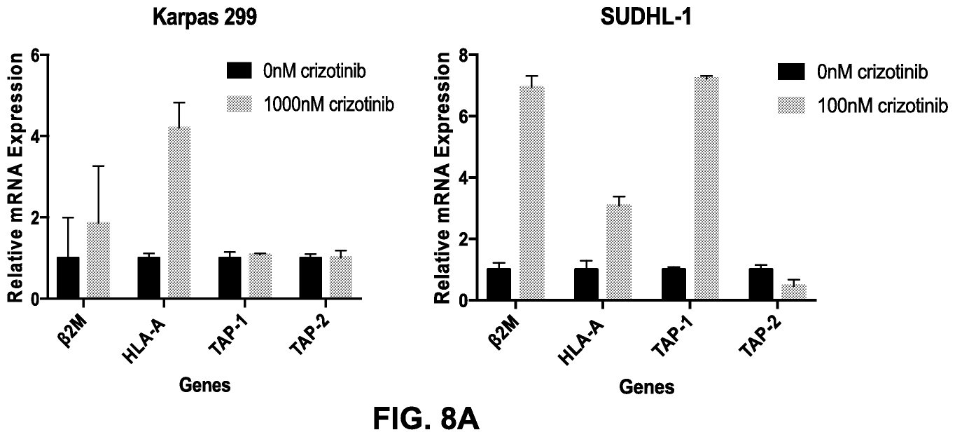

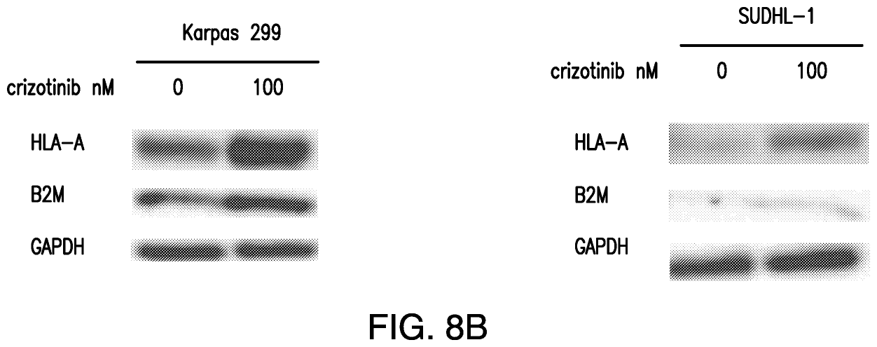

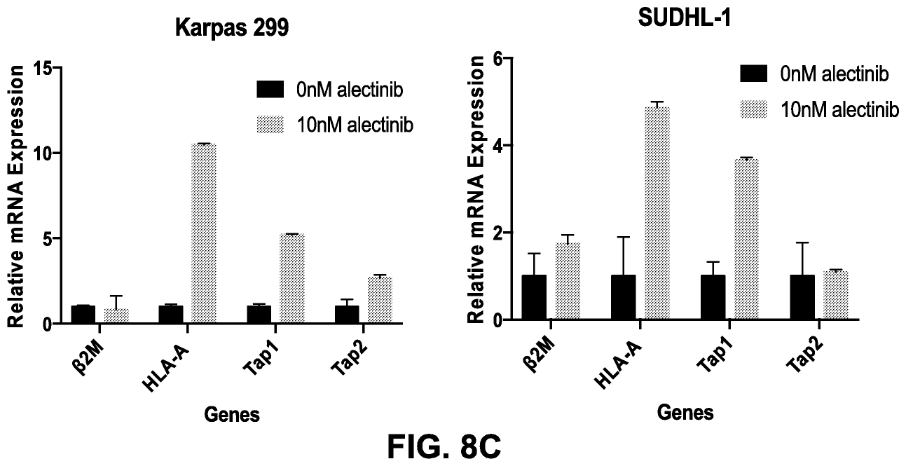

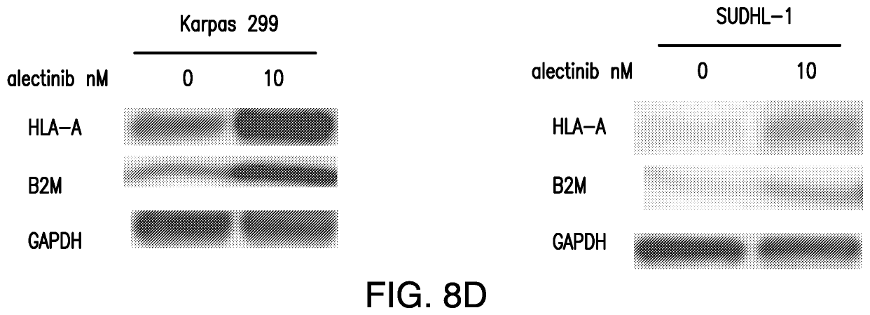

Nascent HLA molecules reside in the endoplasmic reticulum (ER) until the association of beta-2-microglobulin (β2M) and the proper loading of antigenic peptides; after this process, the complex is shuttled to the cell surface and is later recycled back through endosomes. Therefore the increase in net cell surface HLA could have been the result of increased transcription or translation, or increased stabilization by peptide loading and beta-2-microglobulin association. The increase in surface HLA seen from RTK inhibition resulted from an increase in transcript and protein levels of HLA as assayed through qPCR and western blots, respectively, indicating an effect on molecule number (FIGS. 7A-7D; FIGS. 8A-8D). Moreover, there was an increase in transcript levels of antigen processing machinery as well. The increase in TAP1 (transporter 1, ATP binding cassette subfamily B member) and TAP2 (transporter 2, ATP binding cassette subfamily B member), transporters responsible for shuttling proteasome-cleaved peptides into the ER, and beta-2-microglobulin, indicated a potential for more peptide loading in the ER and stabilization of the cell surface HLA. Furthermore, increase in antigen processing machinery can alter the peptides that are processed and displayed.

6.1.3.6. Upregulation of HLA Through RTK Inhibition Acts Through New Pathways Aside from MAPK

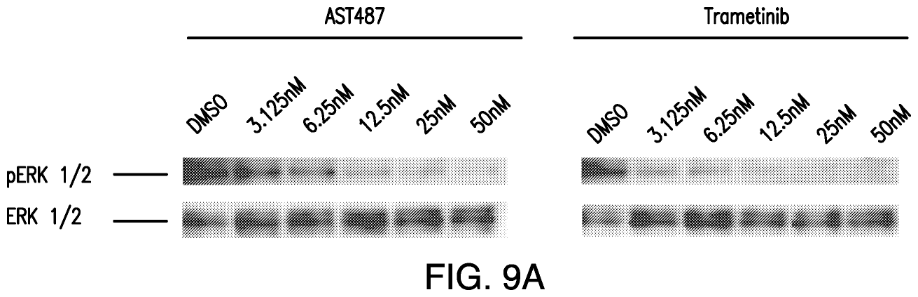

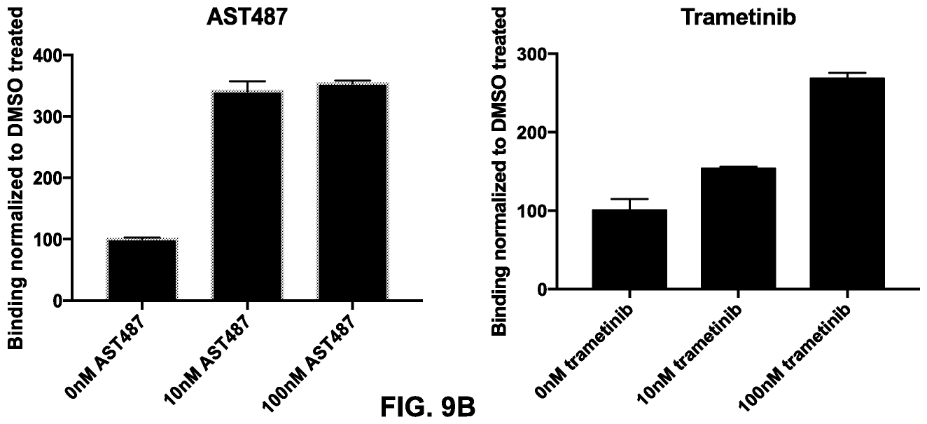

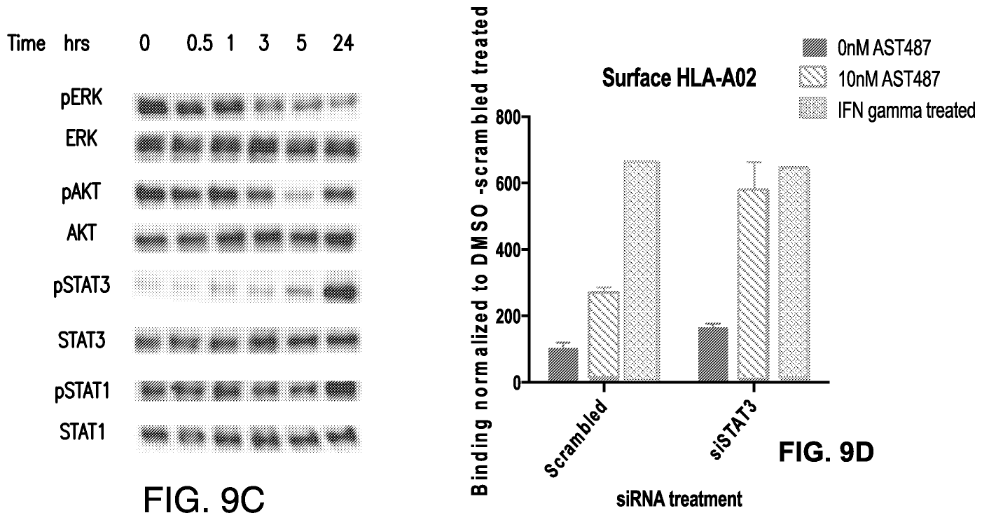

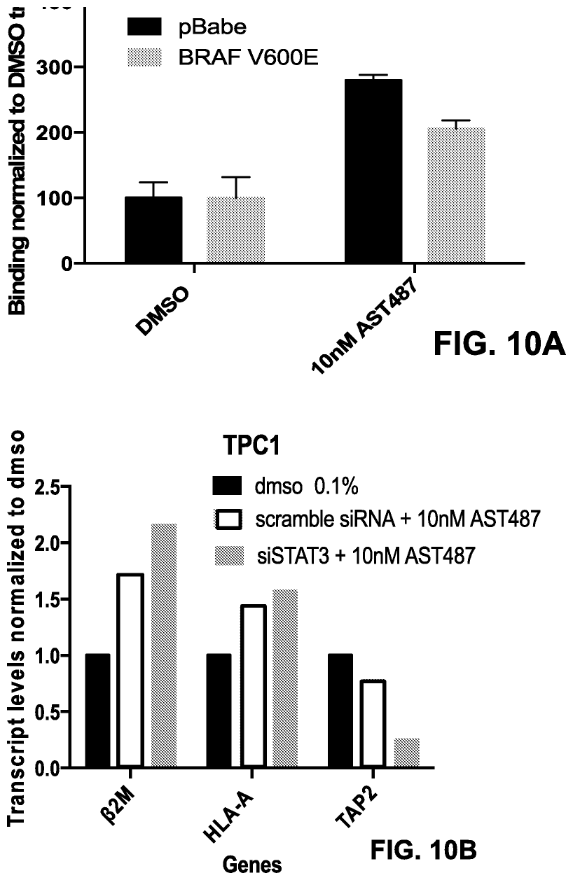

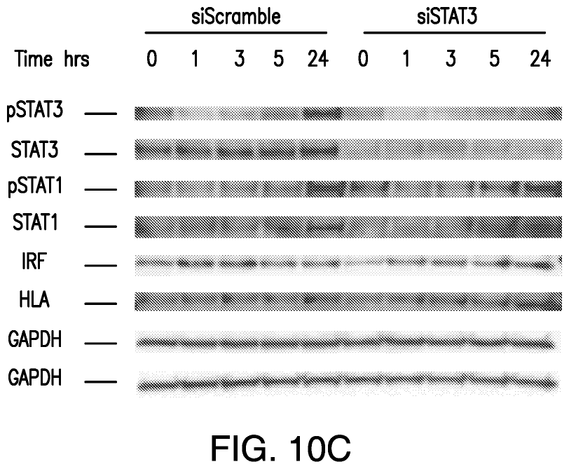

Next, it was analyzed if there could be other pathways involved in HLA upregulation. Since RET is upstream of many additional pathways such as PI3K, JAK-STAT, and PKC, AST487 was used as a tool to block upstream multiple pathways at once. A western blot after 24 hrs of AST487 and trametinib treatment, showed that trametinib shut down pERK at lower doses of drug than AST487 (FIG. 9A). However, AST487 was more potent and increased HLA levels at lower doses of drug (FIG. 9B). This indicated that there could be other pathways at play. To confirm this, a BRAF V600E mutation was introduced into TPC1 cells to induce a constitutively active MAPK pathway. Because RET is upstream of BRAF, inhibition with a RET inhibitor should not shut down pERK expression and if inhibition of the MAPK pathway is the sole pathway in which RET regulates HLA, then RET inhibition should not effect surface HLA expression. The control group (pBabe) had about a 3-fold increase, as normally seen with AST487 treatment at 72 hours. Treatment of BRAF V600E cells with AST487 increased surface HLA about 2-fold, indicating another pathway was involved in regulation (FIG. 10A).

6.1.3.7. Knockdown of STAT3 Prior to RET Inhibition can Increase the Upregulation

To probe the potential pathway involved, the phosphorylation events during the early time points after AST487 treatment were examined (FIG. 9C). Of note was the level of STAT3 phosphorylation that increased significantly with RET inhibition at 24 hrs. RET is upstream of the IL6/JAK/STAT3 pathway and RET/PTC activates the STAT3 by phosphorylation at the tyrosine 705 residue (Hwang et al., 2003, Mol Endocrinol 17(6):1155-1166). Therefore RET inhibition should lead to decreased levels of phosphorylated STAT3 (pSTAT3). Since signaling changes occur within the first few hours, the late onset of phosphorylation lead us to believe the increase in STAT3 activation to be a rebound effect. Treatment with siSTAT3 for 24 hrs before treating with AST487 caused HLA levels to increase almost 6-folds, compared to the 3-fold seen without STAT3 knockdown (FIG. 9D). Transcript levels of beta-2-microglobulin and HLA-A increased slightly with the knockdown of STAT3 as well (FIG. 10B). This indicates the potential negative role of pSTAT3 on the regulation of HLA. STAT1 is known to have a role in HLA regulation and due to the reciprocal nature of STAT1 and STAT3, experiments were performed to make sure that the upregulation of HLA was not due to the increased activation of STAT1 (Zhou, 2009, 28(3-4):239-260; and Avalle et al., 2012, JAKSTAT 1(2):65-72). Knockdown of STAT3 did not cause higher levels of pSTAT1 compared to the control group, while protein levels of HLA started showing increase at 24 hrs, indicating STAT3 as a potential novel regulator of HLA (FIG. 10C). Hence, preventing the phosphorylation of STAT3 that arises after RET could lead to further increases in HLA levels.

6.1.3.8. RET Inhibition Alters the Surface HLA-Peptide Repertoire and Presents Novel Peptides

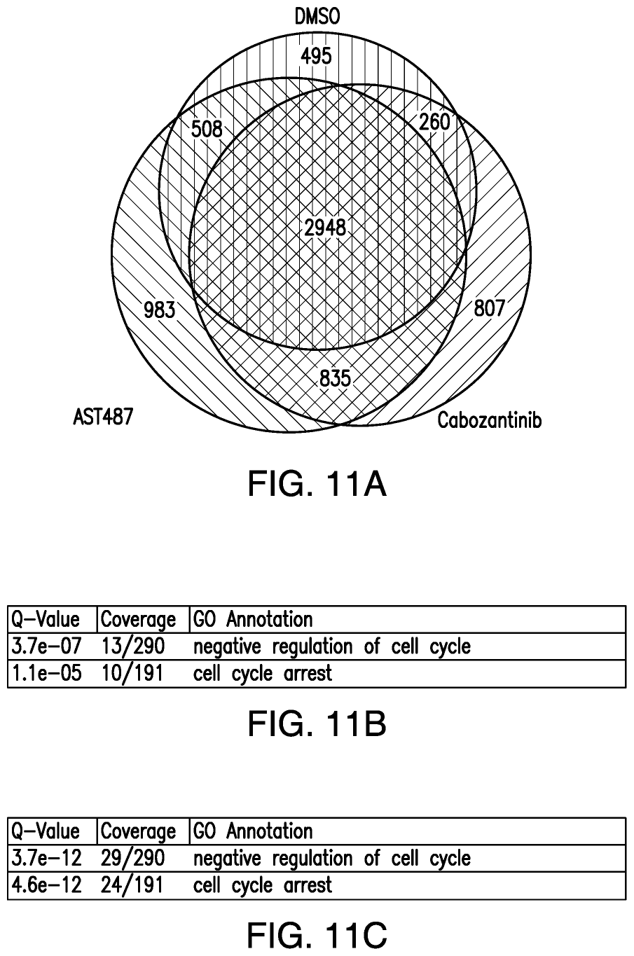

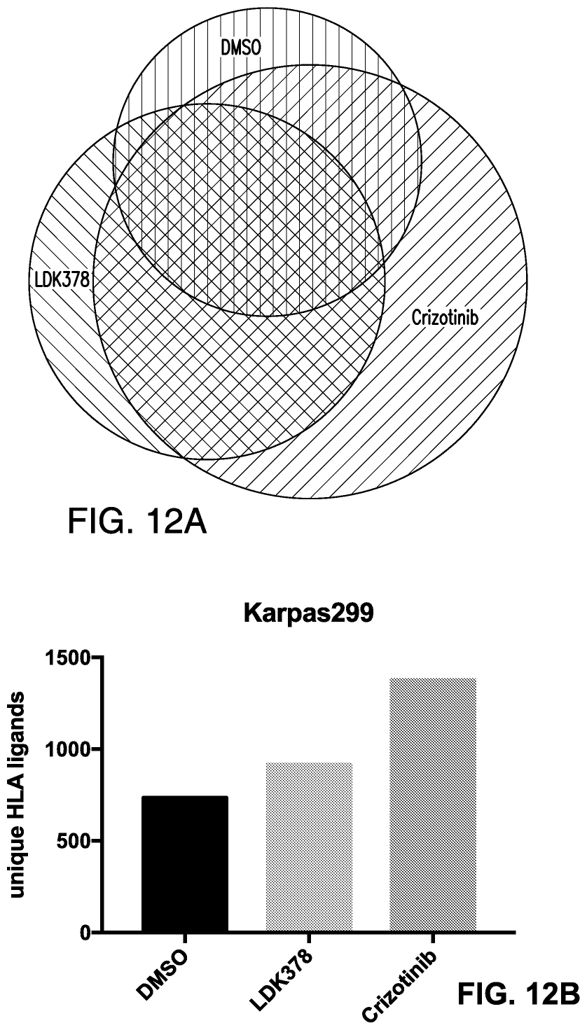

In addition to increases in cell surface HLA and antigen presentation pathways, it is possible that the peptide repertoire was altered with RTK inhibition. This would have a profound impact on the use of these inhibitors as immune adjuvants or therapies. Cells were treated with dimethyl sulfoxide (DMSO), 10 nM AST487, or 100 nM cabozantinib for 3 days and then used pan-HLA antibody bound sephorose column to bind to HLA complexes. Peptides were eluted and mass spectrometry was performed. The venn diagram was used to show the profile of peptides acquired from all three runs, where each count is a unique peptide that was found (therefore repeated peptides in the same or different run was counted only once) (FIG. 11A). Overlap of the circles indicated a peptide that appeared in multiple treatment groups. The control group (red circle) had 4211 unique ligands presented, whereas the AST487 group (purple circle) and cabozantinib group (green circle) had 5274 and 4850 unique ligands presented, respectively. The 25% increase in AST487 unique peptides and 15% increase in cabozantinib unique peptides indicate the presentation of new peptide targets arising after kinase inhibitor treatment. Even more interesting is the 1818 and 1642 unique peptides that were not present in the DMSO group but arose after AST487 and cabozantinib treatment, respectively. Because these peptides were not present in the control cells, this increases the chance that there are some immunogenic peptides in the thousands of new peptides. Moreover, the overlap between unique peptides from the two different RET inhibited groups is double the size of the overlap between either of the RET inhibitors with control, which indicates that altering antigen presentation through kinase inhibition can lead to the peptide repertoire to convene in a similar fashion. Network analysis showed that peptides found in the overlap were most enriched in the cell cycle arrest and negative regulation of the cell cycle pathways (FIGS. 11B-11C). A similar shift in peptide repertoire was seen with ALK inhibition in Karpas 299 (FIGS. 12A-12B). Though the change in unique peptide is lower than change in HLA molecule number, this likely can be accounted for greater presentation of the same peptides as well. Interestingly, network analysis of the DMSO-treated, control peptides showed highest enrichment in the antigen processing and presentation pathway. This may be due to the decrease in degradation of antigen processing components in the kinase-inhibited cells. Due to the upregulation of antigen processing machinery in RET inhibited cells, this indicates an importance of these proteins.

6.1.4. Discussion

This Example shows that inhibition of ALK and RET leads to increased levels of HLA in cells that contain the respective mutant oncogenes. This Example also shows the correlation of these inhibitors shutting down the phosphorylation of ERK with the upregulation of HLA. In combination with T cell based therapies, increased HLA levels can enhance the ability of the T cells to recognize their target. With the plethora of T cell based therapies in the clinic, the ability to increase HLA levels would be useful across a wide variety of treatments. The minimal or absence of expression of ALK and RET on normal cells make these RTKs clinically appealing targets due to the selective HLA upregulation on cancer cells and the decreased side effects from inhibitors deterring essential functions of normal cells. Though these receptors activate the MAPK pathway, this Example shows that they can be involved in HLA regulation through other pathways as well. ALK and RET inhibitors act upstream of multiple pathways and if more than one pathway affecting HLA is inhibited, this can lead to an additive effect on HLA upregulation. For a protein with important immunological function, HLA's regulation is still in the early stages of discovery.

Additionally, this Example shows that the inhibitors of certain kinases can potentially allow for new T cell therapies by uncovering new targets on the surface that arise only after treatment. T cells are tolerized to peptides they constantly see to prevent autoimmune reactions. However, a shift in peptide repertoire could lead to new peptides that are not found in other cells in the body and only on the inhibitor-reactive cancer cells. For instance, with inhibition of proteins upstream of multiple pathways, this can alter the transcription and translation of certain genes and this could potentially include tumor-associated antigens. Further the generation of new peptides could be resulting from altered cleavage patterns of proteins normally presented. Ideally, use of kinase inhibitors, for example, ALK and RET inhibitors, could generate peptides that the T cells are not used to seeing and therefore creating a “personalized neoantigen”.

### 6.2. Example 2: Regulation of HLA Class I Surface Expression Through the Inhibition of BRAF and ERBB2

Experiments in this Example were performed as described in Section 6.1.2.

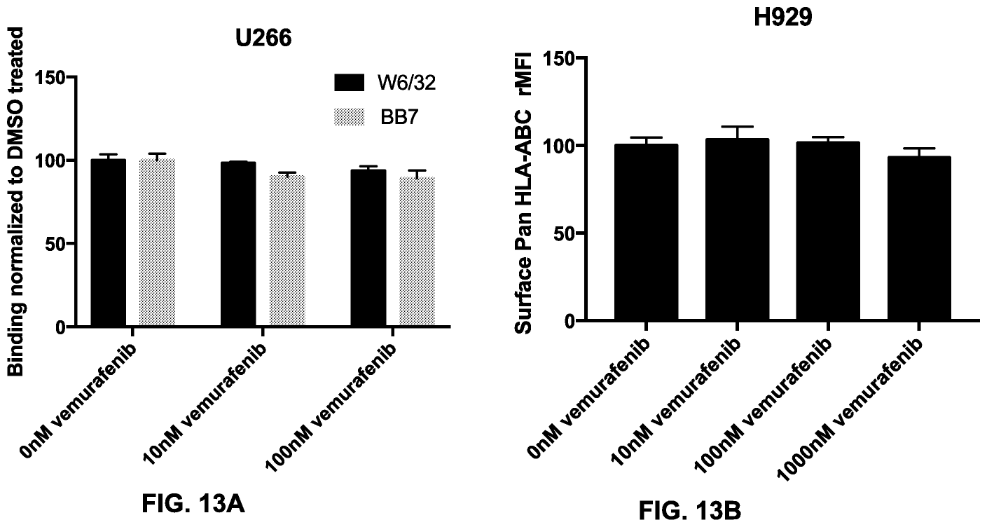

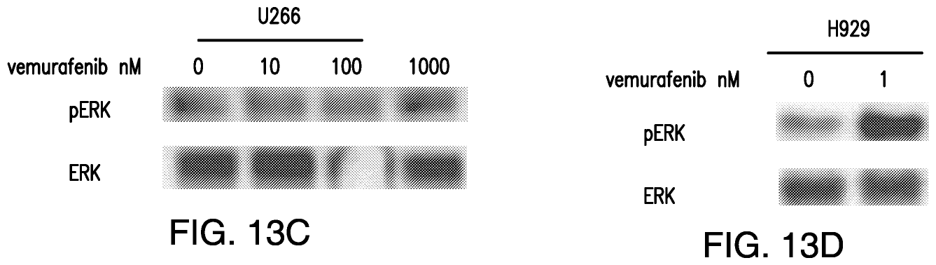

FIGS. 13A-13D show that vemurafenib, a BRAF (B-Raf proto-oncogene, serine/threonine kinase) inhibitor, did not upregulate HLA in BRAF mutant myeloma cell lines.

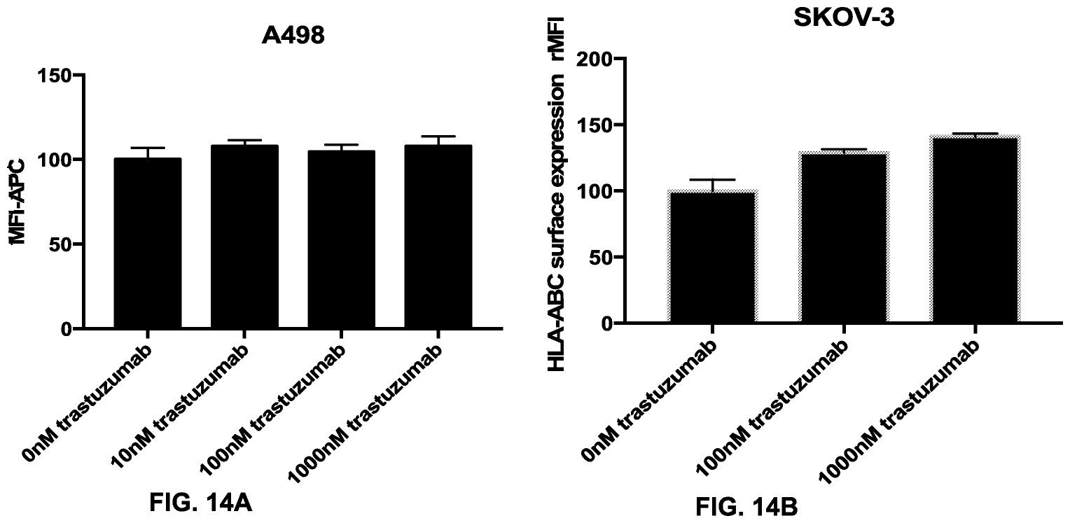

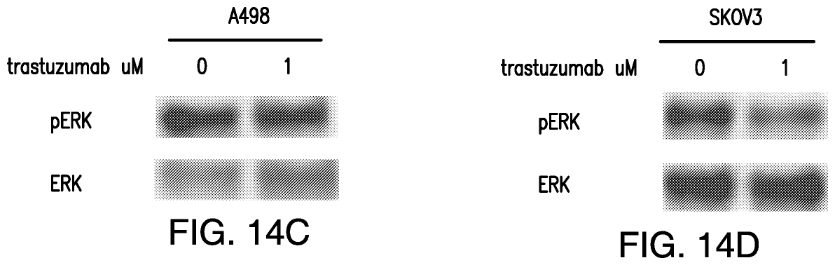

FIGS. 14A-14D show that trastuzumab, an ERBB2 (erb-b2 receptor tyrosine kinase 2) inhibitor, decreased pERK in SKOV3 and not A498 cells, hence HLA upregulation was only seen in SKOV3 cells.

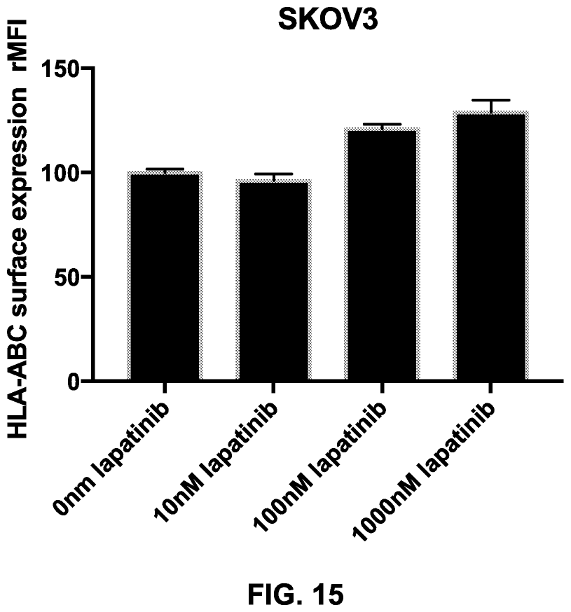

FIG. 15 shows that lapatinib, an ERBB2 inhibitor, upregulated HLA in SKOV3 cells.

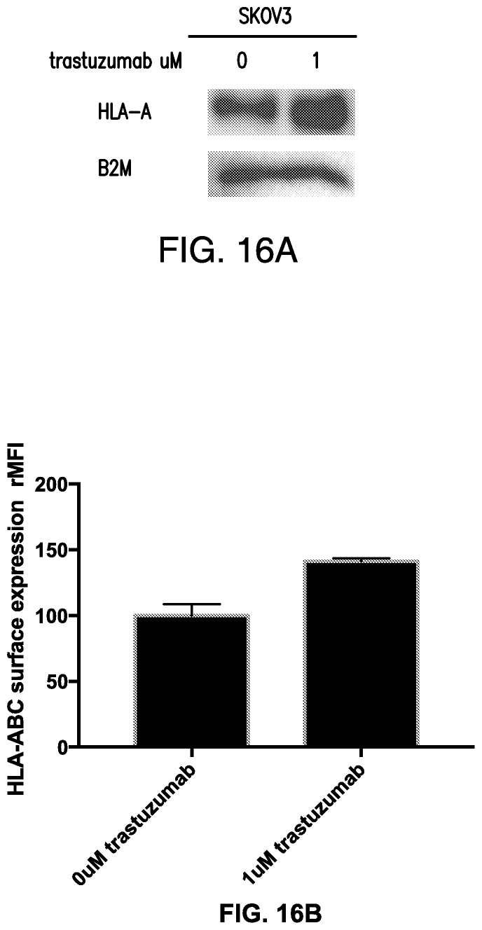

FIGS. 16A-16B show that surface HLA on trastuzumab treated SKOV3 cells could potentially be limited by beta-2-microglobulin protein. This is because HLA presentation requires one beta-2-microglobulin for each HLA molecule on the surface. However, in other cancer cells, where beta-2-microglobulin levels are not limiting, more pronounced changes would be expected.

### 6.3. Example 3: ALK and RET Inhibitors Promote HLA Class I Antigen Presentation and Unmask New Antigens within the Tumor Immunopeptidome

The following Example presents some of the same data as described in Example 1 plus some additional data. This Example is disclosed in Oh et al., 2019, “ALK and RET inhibitors promote HLA class I antigen presentation and unmask new antigens within the tumor immunopeptidome,” Cancer Immunology Research doi: 10.1158/2326-6066.CIR-19-0056 (published in a manuscript form online on Sep. 20, 2019).

6.3.1. Summary

T cell immunotherapies are often thwarted by the limited presentation of tumor-specific antigens abetted by the downregulation of human leukocyte antigen (HLA). This Example shows that drugs inhibiting ALK and RET produced dose-related, increases in cell surface HLA in tumor cells bearing these mutated kinases in vitro and in vivo, as well as elevated transcript and protein expression of HLA and other antigen processing machinery. Subsequent analysis of HLA presented peptides after ALK and RET inhibitor treatment identified large changes in the immunopeptidome with the appearance of hundreds of new antigens, including T cell epitopes associated with impaired peptide processing (TEIPP) peptides. ALK inhibition additionally decreased PD-L1 levels by 75%. Therefore, these oncogenes may enhance cancer formation by allowing tumors to evade the immune system by down regulating HLA expression. Altogether, RET and ALK inhibitors could enhance T cell-based immunotherapies by upregulating HLA, decreasing checkpoint blockade ligands, and revealing new, immunogenic, cancer-associated antigens.

6.3.2. Introduction

Emerging therapies such as checkpoint blockade, CAR T cells, TCR engineered cells, and adoptive T cell transfer have focused attention on the presentation of cancer-associated antigens that are the target of these T cell-based therapies. Though the mechanisms of action behind these therapies vary tremendously, the core component of them is inducing the ability of T cells to kill cancer cells after their T cell receptors (TCRs) recognize the appropriate peptides complexed with human leukocyte antigen (HLA) (Thorsby, 1984, Hum Immunol 9(1):1-7; Rosenberg, 1999, Immunity 10(3):281-287). Peptide/HLA complexes that are recognized trigger a cytolytic response by the T cell (Blum et al., 2013, Annu Rev Immunol 31:443-473; Andersen et al., 2006, Journal of Investigative Dermatology 126(1):32-41). However, the low density surface presentation of tumor-associated peptide/HLA antigens, the lack of immunogenic new antigens, and the ability of some cancers to downregulate the antigen presentation machinery can hinder the ability of T cells to recognize and destroy their target (Demanet et al., 2004, Blood 103(8):3122-3130; Hicklin et al., 1999, Molecular Medicine Today 5(4):178-186; Gejman et al., 2018, elife 7:e41090). Multiple studies, including those performed in lung, melanoma, bladder, and colorectal carcinomas, have shown that up to two-thirds of tissue samples or cell lines harbor alterations in HLA (Campoli and Ferrone, 2008, Oncogene 27(45):5869-5885). These alterations include loss of the entire HLA class I locus, defective antigen presentation machinery (like beta 2-microglobulin mutations), and loss of specific HLA loci (Mcgranahan et al., 2017, Cell 171(6):1259-1271; Mendez et al., 2009, Cancer Immunol Immunother 58(9):1507-1515; Cabrera et al., 2003, Tissue Antigens 62(4):324-327; Maleno et al., 2004, Immunogenetics 56(4):244-253). Thus, cancer cells use downregulation of HLA as a potential mechanism of immune escape (Garrido et al., 2012, Carcinogenesis 33(3):687-693).

It was previously hypothesized that increasing the surface levels of HLA on cancer cells utilizing small molecule drugs could increase both the number and diversity of antigens presented, thereby increasing the efficacy of T cell-based immunotherapies (Brea et al., 2016, Cancer Immunol Res 4(11):936-947). Inhibition of the mitogen-activated protein kinase (MAPK) pathway leads to increased transcript, protein, and surface levels of HLA in a STAT1-mediated manner (Brea et al., 2016, Cancer Immunol Res 4(11):936-947). STAT1 increases HLA by activating transcription of the interferon regulatory factor 1 (IRF1), a transcription factor that binds to a interferon-stimulated response element (ISRE) and activates transcription of HLA-molecules (Gobin et al., 1999, J Immunol 163(3):1428-1434). HLA increase led to amplified cytotoxicity of TCR mimic antibodies to selected epitopes in vitro. However, some MAPK inhibitors are not selective for tumor cells and may cause T cell dysfunction, potentially limiting the effectiveness of this approach (Vella et al., 2013, J Immunother Cancer 1 (Suppl 1):P93; Ebert et al., 2016, Immunity 44(3):609-621; D'Souza et al., 2008, J Immunol 181(11):7617-7629; Dushyanthen et al., 2017, Nat Commun 8(1):606).

Aberrations in the MAPK pathway or kinases that feed into this pathway are involved in the pathogenesis of many cancers. For example, 70% of papillary thyroid cancers have non-overlapping mutations in BRAF, Ras, or RET (REarranged during Transfection) (Menicali et al., 2012, Front Endocrinol (Lausanne) 3:67). Binding of RET ligands and its co-receptor leads to dimerization, autophosphorylation and activation of downstream signaling pathways like MAPK and PI3K (Menicali et al., 2012, Front Endocrinol (Lausanne) 3:67; Knauf and Fagin, 2009, Curr Opin Cell Biol 21(2):296-303; Santoro et al., 1999, Journal of Endocrinological Investigation 22(10):811-819). The most common genetic alteration, RET/PTC1, a fusion of the 3′ portion of RET with the 5′ end of CCDCl6 (Coil coil domain containing 6) (Menicali et al., 2012, Front Endocrinol (Lausanne) 3:67), drives transcriptional activation and constitutive phosphorylation (Knauf et al., 2003, Oncogene 22(28):4406-4412). RET fusions considered capable of oncogenic transformation are seen in about 30% of papillary thyroid cancer and 1-2% of non-small cell lung cancer (NSCLC) (Gainor and Shaw, 2013, Oncologist 18(7):865-875).

ALK is a receptor tyrosine kinase that signals through the MAPK pathway, and that is minimally expressed in adult tissues but mutations leading to expression are seen in a variety of cancers (Hallberg and Palmer, 2013, Nat Rev Cancer 13(10):685-700). An oncogenic fusion protein product of one such fusion, nucleophosmin-anaplastic lymphoma (NPM-ALK) results from the translocation between chromosome 2 and 5 and is found in approximately 75-80% of all ALK positive anaplastic lymphomas (ALCLs) (Webb et al., 2009, Expert Rev Anticancer Ther 9(3):331-356). Homodimers or NPM/NPM-ALK heterodimers lead to constitutive activation of ALK and subsequent activation of downstream signaling pathways like MAPK and PI3K (George et al., 2014, Oncotarget 5(14):5750-5763).

In the study described in this Example, inhibition of both mutant RET and ALK in cancer cells led to downregulation of ERK output and subsequent upregulation of antigen presentation machinery. Large changes in the HLA class I presented peptide ligandome were seen following ALK and RET inhibition, leading to the appearance of hundreds of new T cell epitopes, some of which were immunogenic to human T-cells. Among the new T-cell epitopes found in this study were “impaired peptide processing peptides” (TEIPP), which are predicted to be found only on cells with defects in antigen processing and presentation (Marijt et al., 2018, J Exp Med 215(9):2325-2337; Kiessling, 2016, Journal of Clinical Investigation 126(2):480-482; Lampen et al., 2010, J Immunol 185(11):6508-6517). RNA-Seq and mass spectrometry data gave insight into the changes in gene expression and HLA upregulation that led to this dramatic repertoire shift. Overall, this study demonstrated that the expanded HLA capacity after ALK and RET inhibition gave rise to specific T-cell epitopes that potentially represent new specific targets for immunotherapies.

6.3.3. Materials and Methods

6.3.3.1. Cells Lines, Inhibitors, and Antibodies

The Karpas 299 (HLA-A*03, HLA-A*11), SUDHL-1 (HLA-A*02), and SUP-M2 cells were obtained from the Anas Younes lab at MSKCC and were maintained in RPMI-1640 with 10% FBS and 2 mM L-glutamine. The TPC1 cell line (HLA-A*02, HLA-A*24) was obtained from the James Fagin lab at MSKCC and maintained in DME media with 5% FBS and 2 mM L-glutamine. All obtained cells were tested for Mycoplasma. TT cells and LC-2/ad cells were purchased from ATCC and Sigma-Aldrich, respectively. Cells were maintained in culture 4-12 weeks. Cells were tested for mycoplasma approximately quarterly. For cells not recently purchased, cells were authenticated by flow cytometry of relevant markers and if clonal outgrowths appeared, sorted for purity. TT cells were maintained with Ham's F12 medium supplemented with 10% FBS and 2 mM L-glutamine. LC-2/ad cells were maintained with RPMI-1640: Ham's F12 (1:1) medium supplemented with 10% FBS and 2 mM L-glutamine. ALK inhibitors crizotinib, ceritinib, alectinib, and ruxolitinib were purchased from Selleck Chemicals. The RET inhibitor, AST 487, was purchased from MedChemExpress. Multi-kinase inhibitor Cabozantinib which also targets RET and RAF inhibitor CEP-32496 were purchased from Selleck Chemicals. Antibodies for western blots for phospho-ERK (catalog 4370S), ERK (catalog 4696S), beta-2-microglobulin (catalog 12851S), STAT3 (4904S), pSTAT3 (catalog 9131S), STAT1 (catalog 9175S), pSTAT1 (catalog 9167S) and GAPDH (catalog 3683S) were purchased from Cell Signaling. Antibodies for western blot for anti-HLA-A (catalog sc-23446), goat anti-mouse IgG-HRP, mouse anti-rabbit IgG-HRP, anti-CD30, and donkey anti-goat IgG-HRP were purchased from Santa Cruz. Antibodies for flow cytometry to HLA-A02 (BB7.2) and HLA-A,B,C (W6/32) were purchased from eBioscience; the PD-L1 (MIH1) antibody was purchased from eBioscience; ESK1 and hIgG1 (catalog ET901) were provided by Eureka Therapeutics.

6.3.3.2. Flow Cytometry

5×10⁴ cells were seeded in a 12 well plate and treated with drug for 72 hrs. Adherent cells were seeded one day before treatment. At 72 hrs, cells were harvested, washed with PBS and incubated on ice with appropriate fluorophore conjugated antibodies diluted in FACS buffer for 1 hr. Gating strategy: Cells were then washed and incubated 30 min with a viability dye (propidium iodide at 1 ug/mL) and live cells only were analyzed by on a Guava flow cytometer with FlowJo software.

6.3.3.3. Western Blots

Cells were seeded in a 60 mm dish or 6 well plates. After the appropriate time point, cells were harvested, lysed in RIPA buffer (Thermo Instruments) and protein concentration was quantified by a Lowry assay (using the Bio-Rad DC Protein Assay; #5000116 on a Spectramax device from Molecular Devices). Protein loading levels were equalized per lane and separated on SDS gels (Bio-Rad). Protein was transferred to a nitrocellulose membrance using semi-dry transfer (BioRad). Membranes were blocked with Omniblock (American Bio) and incubated with respected antibodies. When needed, secondary antibodies with HRP were incubated for 1 hr. Enhanced chemiluminescent substrate for HRP enzymes was used to image protein levels (Thermo-Fischer; #34095) on a ChemiDoc MP imager with Soft Max Pro software (Biorad).

6.3.3.4. Real Time PCR

In brief, cells were treated and incubated with appropriate small molecule inhibitors for 48 hrs and RNA was extracted using Qiagen RNA Easy Plus (Qiagen; #74134). cDNA was created using qScript cDNA SuperMix (Quantabio; #95048). qPCR was performed using PerfeCTa FastMix II (Quantabio; #95118) and TaqMan real time probes purchased from Life Technologies: HLA-A (Hs01058806_g1), beta-2 microglobulin (Hs00187842_m1), TAP1 (Hs00388677_m1), TAP2 (Hs00241060_m1), and TBP (Hs00427620_m1). Data were normalized to baseline expression of each analyzed gene separately. For details see Chang et al., 2017, J Clin Invest 127(7):2705-2718.

6.3.3.5. Antibody-Dependent Cellular Cytotoxicity

For each experiment reported, fresh peripheral blood mononuclear cells were derived from a healthy donor by Ficoll density centrifugation after receiving informed consent on Memorial Sloan Kettering Institutional Review Board-approved protocols. TPC1 cells were treated with DMSO or 10 nM AST487 for 72 hrs to increase surface HLA, after which cells were thoroughly washed in PBS to remove the drugs. Cells were then labeled with 1 uCi/well chromium-51 for 1 hr at 37° C. Chromium labeled TPC1 cells were co-cultured with PBMCs and ESK1 (a human IgG1 reactive with WT1 peptide/HLA-A*02:01) or its isotype control (hIgG1). Different effector:target ratios were used and after 5 hrs of incubation at 37° C., the supernatant was harvested and chromium levels were measured through standard chromium-51 release assay on a Top Count machine (Perkin Elmer).

6.3.3.6. RNA-seq

For RNA-seq analysis total RNA was extracted using the RNeasy Mini Kit (Qiagen) after treatment of TPC-1 cells with either DMSO, AST487 or Cabozantinib for 72 hours. Purified polyA mRNA was subsequently fragmented, and first and second strand cDNA synthesis performed using standard Illumina mRNA TruSeq library preparation protocols. Double stranded cDNA was subsequently processed for TruSeq dual-index Illumina library generation. For sequencing, pooled multiplexed libraries were run on a HiSeq 2500 machine on RAPID mode. Approximately 10 million 76 bp single-end reads were retrieved per replicate condition. Resulting RNA-Seq data was analyzed by removing adaptor sequences using Trimmomatic, aligning sequencing data to GRCh37.75(hg19) with STAR, and genome wide transcript counting using HTSeq to generate a RPKM matrix of transcript counts. This RPKM matrix was further log (log 2) transformed and normalized per gene to obtain the Z-score. Differential gene expression was analyzed by looking at fold changes between experimental conditions.

6.3.3.7. Animal Studies

All animal experiments were conducted in accordance with and the approval of the MSKCC IACUC (Institutional Animal Care and Use Committee) protocols. Female NSG (NOD. Cg-Prkdc^{scid}Il2rg^{tm1Wj1}/SzJ) and NRG (NOD.Cg-Rag1^{tm1Mom}Il2rg^{tm1Wj1}/SzJ) mice were purchased from the Jackson Laboratory at 5-10 weeks old. For experiments in vivo with RET and ALK, 2.5-6×10⁶ tumor cells in PBS were subcutaneously injected into the flank of mice. When tumors were palpable (2-3 mm), mice were treated daily with drugs or vehicles through oral gavage of drugs in 200 ul of water. At day 4 or 7, tumors were harvested and flow cytometry was conducted to determine the effect of inhibitors on HLA and PD-L1 on the tumor cells. N=5 for all treatment groups in vivo (one outlier was excluded from the vehicle group in the RET experiment, but results remained significant: P value with outlier included 0.031, P value without outlier 0.016)). TPC1 cells were transduced with luciferase and GFP on an SFG vector and this allowed gating of the tumor cells in flow cytometry. A CD30 antibody was used in the ceritinib experiments.

6.3.3.8. Immunopurification of HLA Class I Ligands.

Immunopurification affinity columns were prepared as described previously (Bassani-Sternberg et al., 2015, Mol Cell Proteomics 14(3):658-673). In brief, 40 mg of Cyanogen bromide-activated-Sepharose® 4B (Sigma-Aldrich, Cat #C9142) was activated with 1 mM hydrochloric acid (Sigma-Aldrich, Cat #320331) for 30 min. Subsequently, 0.5 mg of W6/32 antibody (BioXCell, BE0079; RRID: AB 1107730) was coupled to sepharose in presence of binding buffer (150 mM sodium chloride, 50 mM sodium bicarbonate, pH 8.3; sodium chloride: Sigma-Aldrich, Cat #S9888, sodium bicarbonate: Sigma-Aldrich, Cat #56014) for at least 2 hours at room temperature. Sepharose was blocked for 1 h with glycine (Sigma-Aldrich, Cat #410225). Columns were equilibrated with PBS for 10 min. TPC1 cells were treated with DMSO, 10 nM AST487, or 100 nM cabozantinib. Karpas 299 cells were treated with DMSO, 100 nM crizotinib or 100 nM ceritinib for 72 h. 20-30×10⁶ cells were harvested and washed three times in ice-cold sterile PBS (Media preparation facility MSKCC). Afterwards, cells were lysed in 1 ml 1% CHAPS (Sigma-Aldrich, Cat #C3023) in PBS, supplemented with 1 tablet of protease inhibitors (cOmplete, Cat #11836145001) for 1 hour at 4° C. This lysate was spun down for 1 hour at 20,000 g at 4° C. Supernatant was run over the affinity column through peristaltic pumps at 1 ml/min overnight at 4° C. Affinity columns were washed with PBS for 15 min, run dry, and HLA complexes subsequently eluted three times with 200 μl 1% trifluoracetic acid (TFA, Sigma/Aldrich, Cat #02031). For separation of HLA ligands from their HLA complexes tC18 columns (Sep-Pak tC18 1 cc VacCartridge, 100 mg Sorbent per Cartridge, 37-55 μm Particle Size, Waters, Cat #WAT036820) were prewashed with 80% acetonitrile (ACN, Sigma-Aldrich, Cat #34998) in 0.1% TFA and equilibrated with two washes of 0.1% TFA. Samples were loaded, washed again with 0.1% TFA and eluted in 400 μl 30% ACN in 0.1% TFA. Sample volume was reduced by vacuum centrifugation for mass spectrometry analysis.

6.3.3.9. LC-MS/MS Analysis of HLA Ligands

Samples were analyzed by a high resolution/high accuracy LC-MS/MS (Lumos Fusion, Thermo Fisher). Peptides were desalted using ZipTips (Sigma Millipore, Cat. #ZTC18S008) according to manufactures instructions and concentrated using vacuum centrifugation prior to being separated using direct loading onto a packedin-emitter C18 column (75 um ID/12 cm, 3 μm particles, Nikkyo Technos Co., Ltd. Japan). The gradient was delivered at 300 nl/min increasing linear from 2% Buffer B (0.1% formic acid in 80% acetonitrile)/98% Buffer A (0.1% formic acid) to 30% Buffer B/70% Buffer A, over 70 minutes. MS and MS/MS were operated at resolutions of 60,000 and 30,000, respectively. Only charge states 1, 2 and 3 were allowed. 1.6 Th was chosen as the isolation window and the collision energy was set at 30%. For MS/MS, the maximum injection time was 100 ms with an AGC of 50,000.

6.3.3.10. Mass Spectrometry Data Processing

Mass spectrometry data was processed using Byonic software (version 2.7.84, Protein Metrics, PaloAlto, Calif.) through a custom-built computer server equipped with 4 Intel Xeon E5-4620 8-core CPUs operating at 2.2 GHz, and 512 GB physical memory (Exxact Corporation, Freemont, Calif.). Mass accuracy for MS1 was set to 10 ppm and to 20 ppm for MS2, respectively. Digestion specificity was defined as unspecific and only precursors with charges 1, 2, and 3 and up to 2 kDa were allowed. Protein FDR was disabled to allow complete assessment of potential peptide identifications. Oxidization of methionine, N-terminal acetylation, phosphorylation of serine, threonine and tyrosine were set as variable modifications for all samples. All samples were searched against the UniProt Human Reviewed Database (20,349 entries, uniprot.org, downloaded June 2017). Peptides were selected with a minimal log prob value of 2 resulting in a 1% false discovery rate and were HLA assigned by netMHC 4.0 with a 5% rank cutoff.

6.3.3.11. Peptide Stimulation

Peripheral blood mononuclear cells were again derived from healthy donors after receiving informed consent (see above). T cells were isolated by Ficoll density centrifugation and stimulated with pools of peptides that were selected from the population of: (1) new peptides that appeared after RET inhibitor treatment, (2) peptides found on cells before RET inhibitor treatment, and (3) irrelevant peptides not found on the target cells. CD14⁺ cells were isolated from PBMCs by negative immunomagnetic cell separation using an isolation kit (Miltenyi Biotec). CD14 cells were used for stimulation in week one and autologous dendritic cells were generated for use thereafter. The purity of the cells was always more than 98%. Monocyte-derived dendritic cells (DCs) were generated from CD14+ cells, by culturing the cells in RPMI 1640 medium supplemented with 1% AP, 500 units/mL recombinant IL-4, and 1,000 units/mL GM-CSF. On days 2 and 4 of the incubation, fresh medium with IL-4 and GM-CSF was either added or replaced half of the culture medium. On day 6, maturation cytokine cocktail was added (IL-4, GM-CSF, 500 IU/mL IL-1, 1,000 IU/mL IL-6, 10 ng/ml TNF-α, and 1 ug/mL PGE-2).

An interferon-gamma ELISpot was performed at the beginning of week three. For this HA-Multiscreen plates (Millipore) were coated with 100 uL of mouse anti-human IFN-gamma antibody (10 ug/mL; clone 1-D1K, Mabtech #3420-2A) in PBS, incubated overnight at 4° C., washed with PBS to remove unbound antibody, and blocked with RPMI 1640/10% autologous plasma (AP) for 2 h at 37° C. Purified CD3⁺ T cells (>98% pure) were plated with either autologous CD14⁺ (10:1 E:APC ratio) or autologous DCs (30:1 E:APC ratio), Various test peptides were added to the wells at 20 ug/mL. Negative control wells contained. APCs and T cells without peptides or with irrelevant peptides. Positive control wells contained T cells plus APCs plus 20 ug/mL phytohemagglutinin (PHA, Sigma). All conditions were done in triplicates. Microtiter plates were incubated for 20 h at 37° C. and then extensively washed with PBS/0.05% Tween and 100 uL/well biotinylated detection antibody against human IFN-γ (2 ug/mL; clone 7-B6-1; Mabtech) was added. Plates were incubated for an additional 2 h at 37° C. and spot development was done as described. Spot numbers were read and determined by Zellnet Consulting In. T cell killing of target cells was measured at week five as described in the section of ADCC above.

6.3.3.12. Statistics

P values were calculated with GraphPad Prism 7 using an unpaired t test for flow cytometry experiments, RNA quantitation and in vivo experiments. Error bars indicate SEM for in vivo experiments and SD for flow cytometry and RNA quantitation experiments. All flow cytometry and RNA quantitation experiments were performed in technical triplicates and with a minimum of 2 biological replicates. Western blots were done at least two-three times. Representative demonstration blots are shown only. Values are reported in figures with “*” equal to P≤0.05, “*” equal to P≤0.01, “***” equal to P≤0.001, and “****” equal to P≤0.0001. No symbol indicates not statistically significant (P>0.05).

6.3.4. Results

6.3.4.1. ALK Inhibition Increased HLA Expression Through MAPK Pathway Suppression

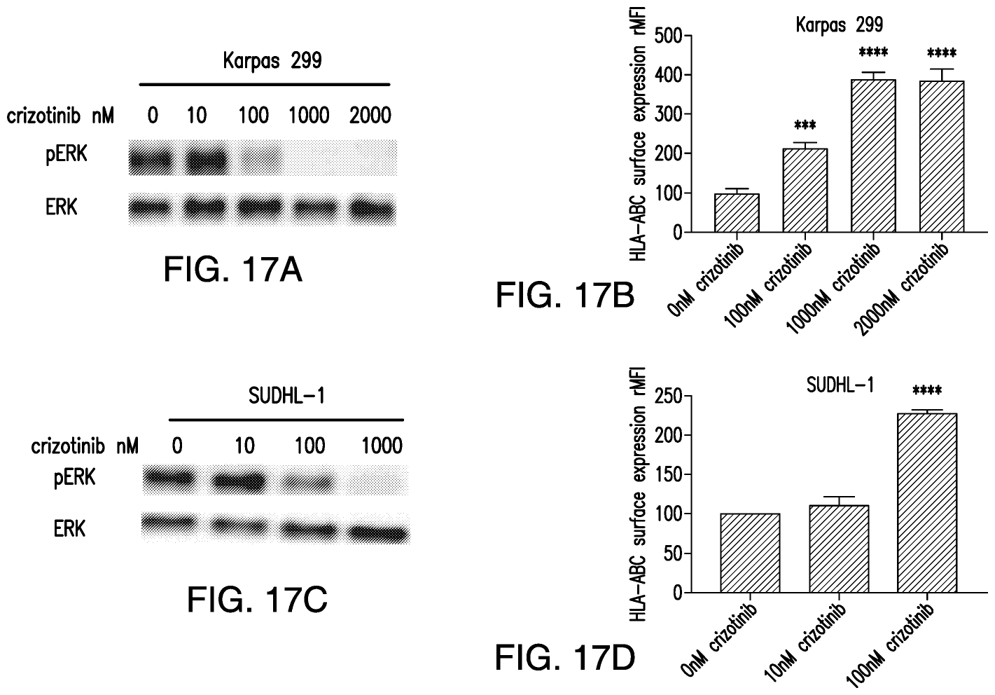

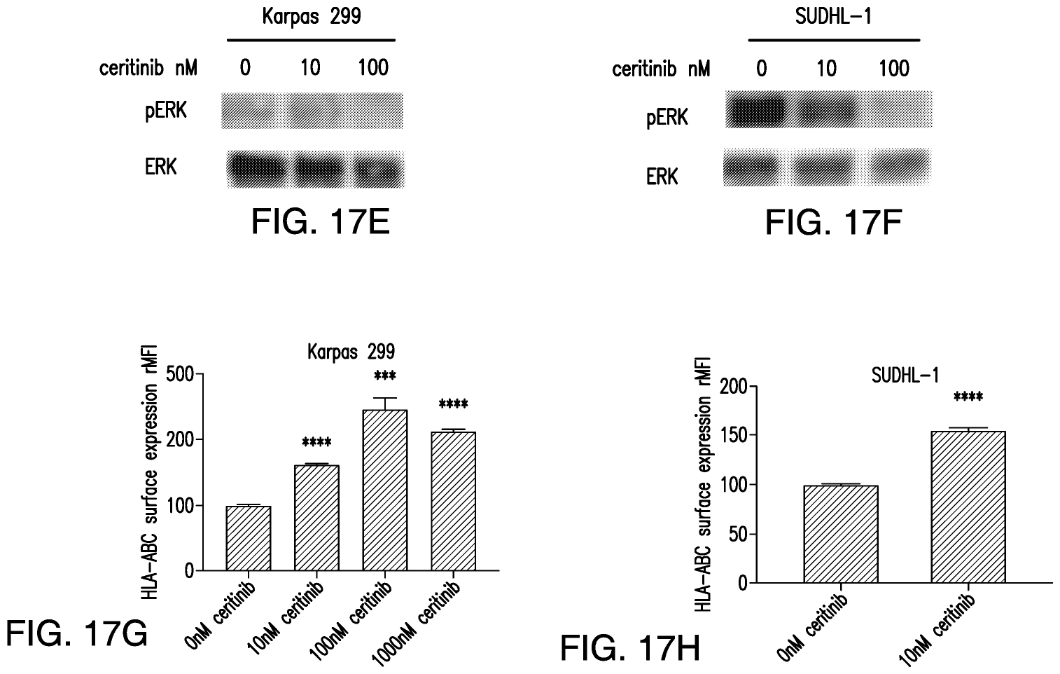

Crizotinib is a small molecule tyrosine kinase inhibitor that is Food and Drug Administration (FDA) approved for the treatment of mutated ALK positive non-small cell lung cancer (NSCLC) (Awad and Shaw, 2014, Clin Adv Hematol Oncol 12(7):429-439). Increasing concentrations of crizotinib on Karpas 299, a NPM-ALK fusion oncogene-positive ALCL cell line, showed a dose-related, reduction of pERK at 3 hours of treatment, indicating inhibition of ALK shuts down the MAPK pathway (FIG. 17A). Flow cytometric analysis of HLA levels after a 72 hour incubation of Karpas 299 cells with crizotinib showed an inverse dose-response. Decreasing levels of pERK were associated with increased levels of surface HLA class I complexes (FIG. 17B). HLA levels on Karpas 299 lymphoma cells treated with 1 uM crizotinib increased 4-fold compared to control cells treated with DMSO. The plateau in surface HLA upregulation that was seen at higher concentrations of crizotinib correlated with complete shut-down of ERK phosphorylation at lower doses. Similar results were seen with SUDHL-1, another NPM-ALK mutated fusion protein positive ALCL line (FIGS. 17C and 17D).

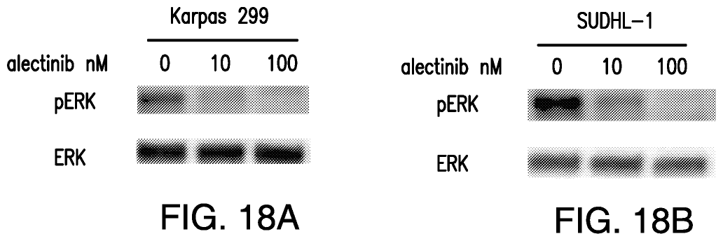

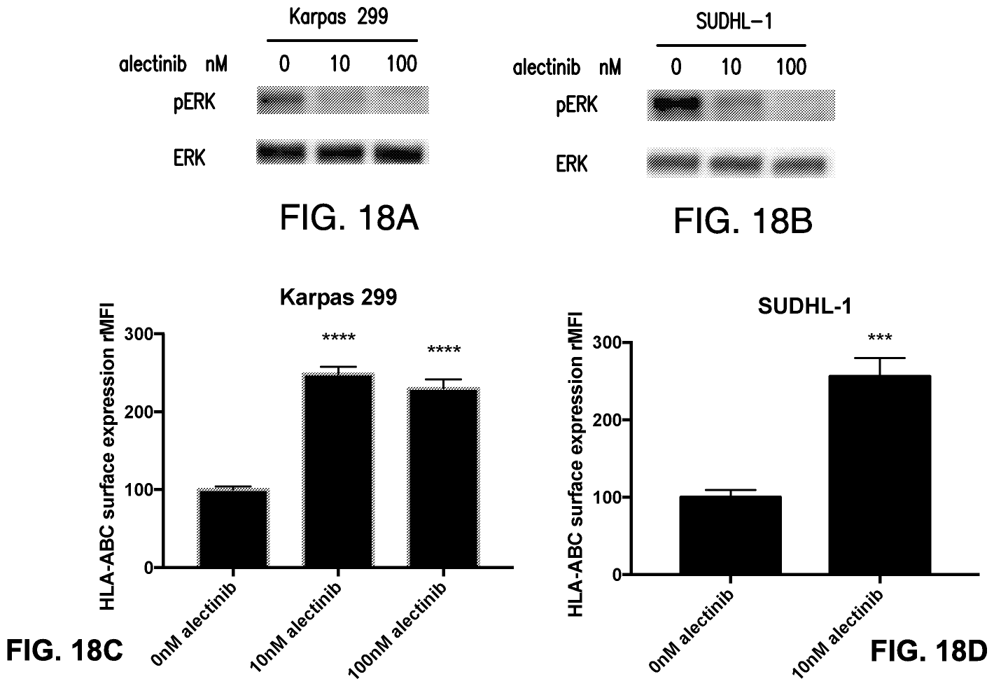

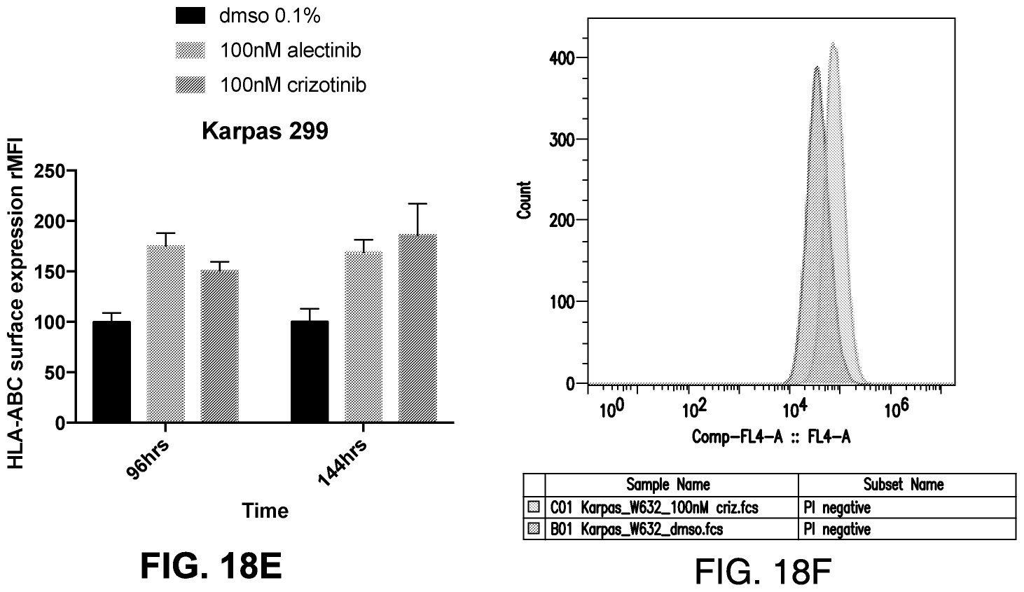

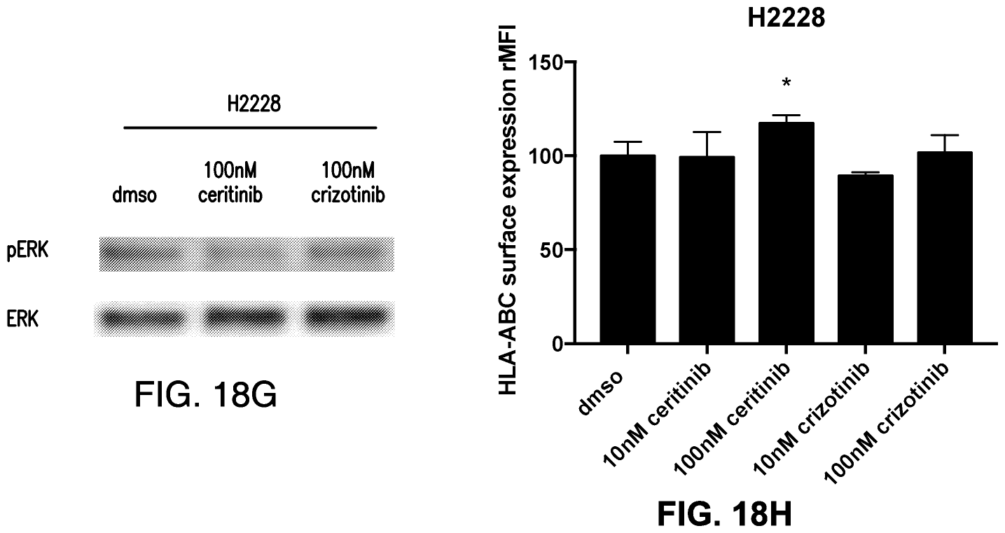

To confirm that ALK inhibition was the mechanistic target for HLA regulation, another small molecule ALK inhibitor, ceritinib (LDK378), which is approved for crizotinib-resistant NSCLC, was investigated (Sullivan and Planchard, 2016, Ther Adv Med Oncol 8(1): 32-47). Treatment of Karpas 299 and SUDHL-1 with increasing concentrations of ceritinib also shut down pERK levels (FIGS. 17E and 17F). Cells were comparatively more sensitive to ceritinib than crizotinib; however, cell surface HLA increased in both cell lines in a dose-dependent manner (FIGS. 17G and 17H). Similar results were seen with alectinib, another second-generation ALK inhibitor (FIGS. 18A-18D). It was also shown that the effect on HLA is temporary, but increased HLA levels were still seen 6 days later, indicating its potential for clinical use (FIG. 18E). A representative flow cytometry histogram of the HLA increases is also provided as an example of the typical raw data (FIG. 18F). As these three inhibitors share ALK as a target, but have different off-target kinases, similar results with the three inhibitors in two different cell lines provided strong confidence that the increase in HLA seen was a result of ALK inhibition and not another kinase (Sullivan and Planchard, 2016, Ther Adv Med Oncol 8(1): 32-47; Shaw et al., 2014, N Engl J Med 370(13):1189-1197; Kodama et al., 2014, Mol Cancer Ther 13(12):2910-2908). The dependence on MAPK inhibition for HLA upregulation was further confirmed with the EML4-ALK protein positive fusion cell line H2228. In these cells, crizotinib did not change pERK levels, and consequently cell surface HLA levels did not change either. Ceritinib resulted in minimal changes in pERK levels and only a minimal increase in surface HLA levels was seen (FIGS. 18G and 18H). Overall, using several inhibitors of ALK in multiple cell lines, the inhibition of ERK output by the drugs correlated positively with cell surface HLA levels.

6.3.4.2. RET Inhibition Increased HLA Expression Through MAPK Pathway Suppression

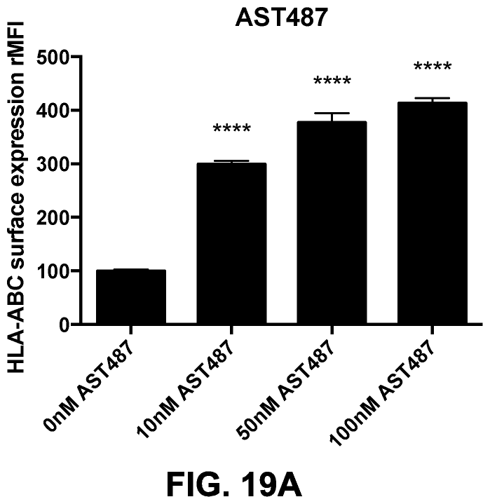

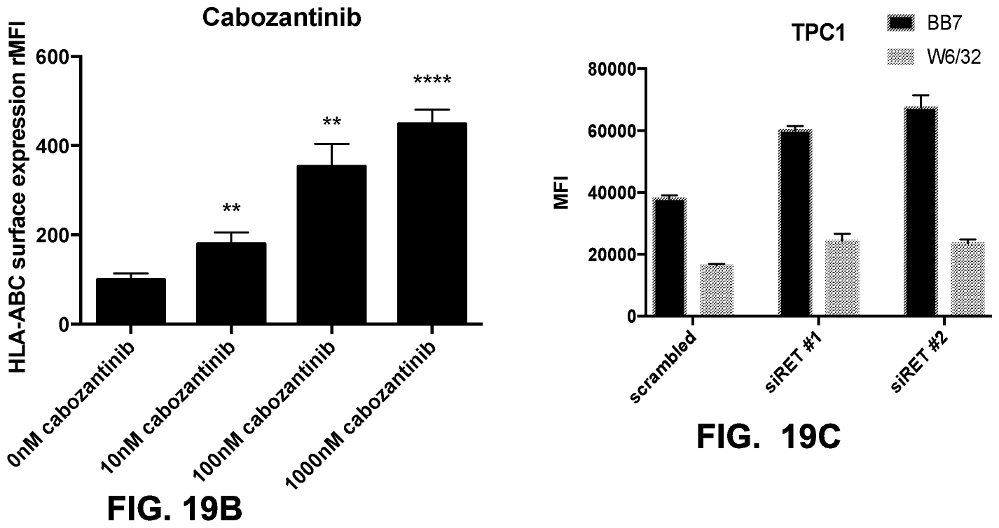

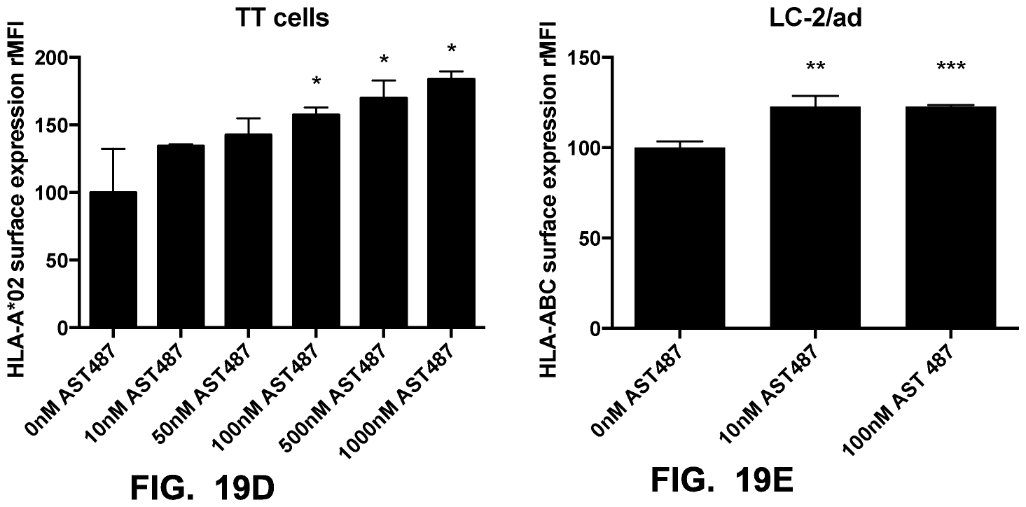

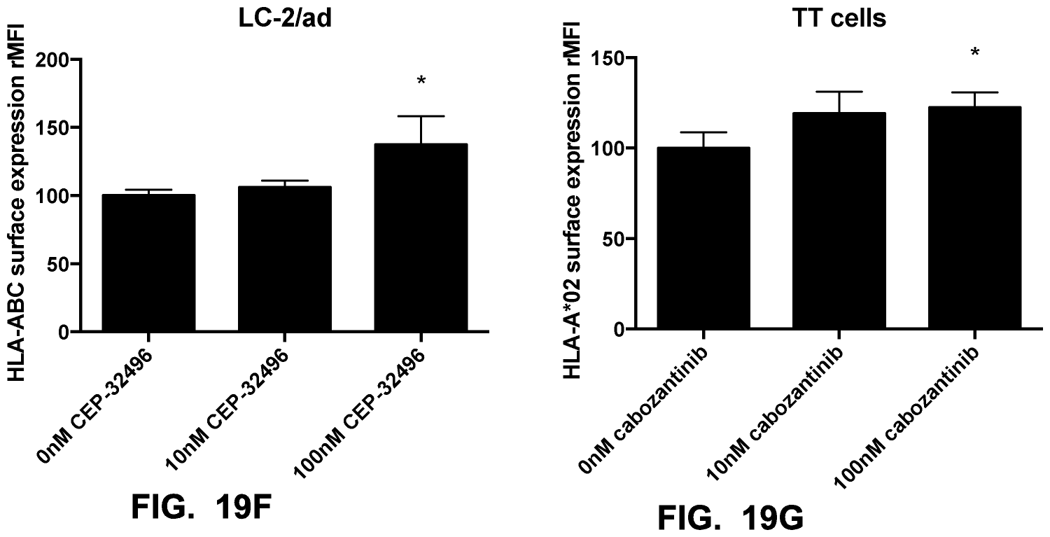

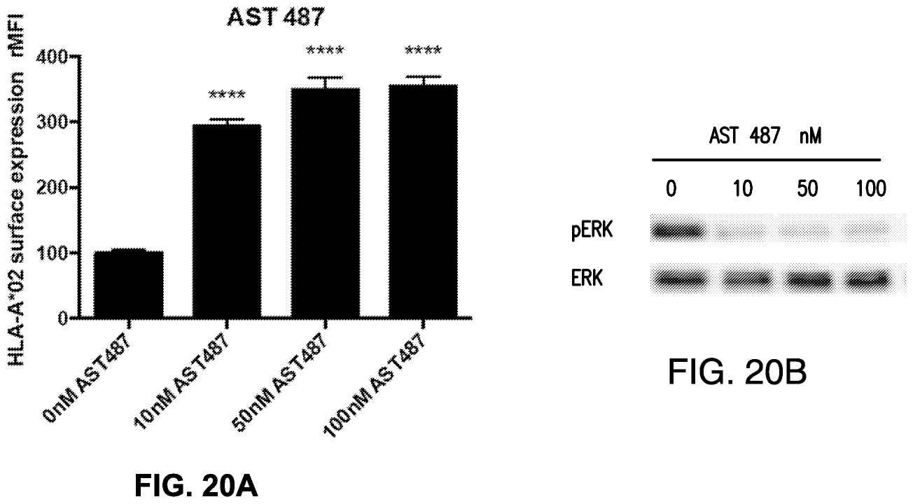

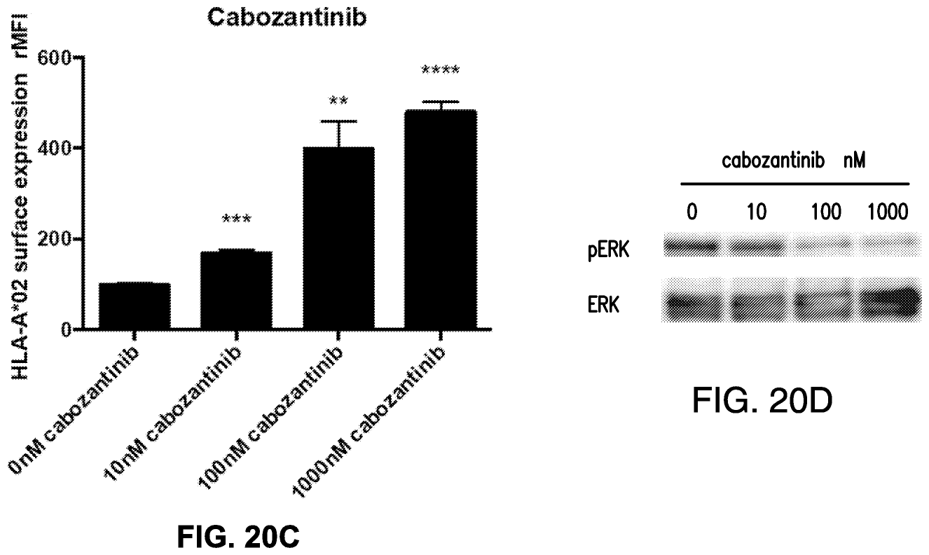

RET is a receptor tyrosine kinase that signals through the MAPK pathway. Specific targeting of RET with AST487 inhibits growth of thyroid cell lines with activating RET mutations, such as TPC1 (Akeno-Stuart et al., 2007, Cancer Res 67(14):6956-6964). Treatment of TPC1 cells with AST487 for 72 h led to a 3 to 4-fold increase in cell surface HLA class I levels (FIG. 19A). In addition to pan-HLA increases, this study investigated HLA upregulation of one of the most common HLA alleles, HLA-A*02:01, which was also increased (FIG. 20A). Inhibition of pERK was seen even at concentrations as low as 10 nM AST487 (FIG. 20B).

Another small molecule kinase inhibitor of RET showed similar effects. Cabozantinib, a small molecule inhibitor of RET, MET, and VEGF2 that is also FDA approved for treatment of medullary thyroid cancer, was tested on TPC1 cells in the same manner (Grulich, 2014, Recent Results Cancer Res 201:207-214). The inhibitor at 100 nM led to a 4-fold increase in surface pan-HLA, as well as HLA-A*02 specifically. Again, a dose response relationship was seen with increasing concentrations of drug. Western blot analysis confirmed decreasing pERK levels with inhibitor treatment (FIGS. 19B, 20C and 20D). By use of these different RET inhibitors, it was confirmed that inhibition of RET was most likely causing the increase in HLA (Akeno-Stuart et al., 2007, Cancer Res 67(14):6956-6964; Grulich, 2014, Recent Results Cancer Res 201:207-214). To further validate that RET inhibition increased HLA, siRNAs were applied to knockdown RET expression. Knockdown of RET by 2 different siRNAs resulted in increased HLA expression (FIG. 19C).

To determine if these findings were reproduced with other RET mutations or in other cancer types, a lung cancer cell line LC-2/ad, which harbors the same CCDCl6-RET fusion as TPC1 (Matsubara et al., 2012, J Thorac Oncol 7(12):1872-1876), was tested. TT cells, which are a medullary thyroid cell line that is driven by a C634W mutation leading to dimerization and activation (Carlomagno et al., 1995, Biochem Biophys Res Commun 207(3):1022-1028), were also examined. HLA levels of the TT line increased in a AST487 dose-related manner (FIG. 19D). In the other line, only small increases were seen with RET inhibition (FIG. 19E). Use of cabozantinib and CEP-32496 also had minimal effects on HLA upregulation (FIGS. 19F and 19G) in these 2 lines. Due to more robust upregulation in TPC1, these cells were used for other RET inhibition studies.

6.3.4.3. ALK and RET Inhibition Increased HLA Through Alterations in Transcript and Protein Expression

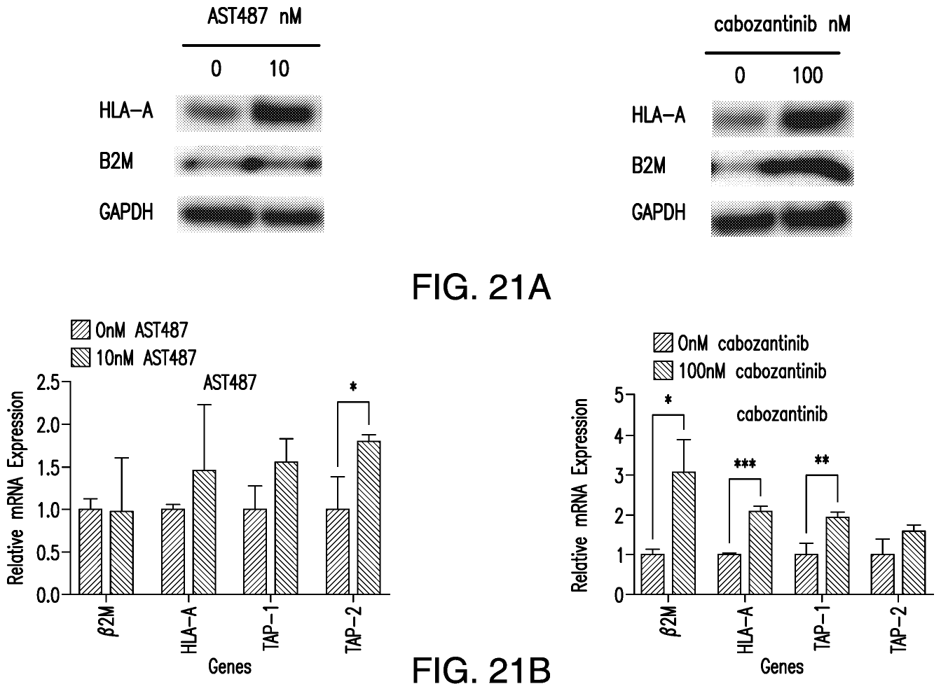

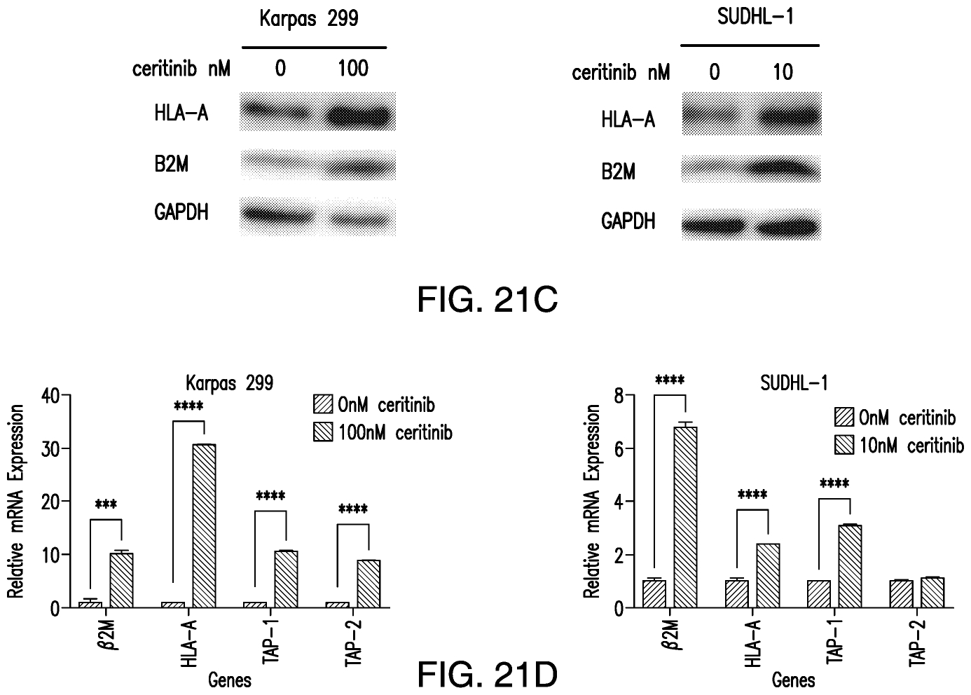

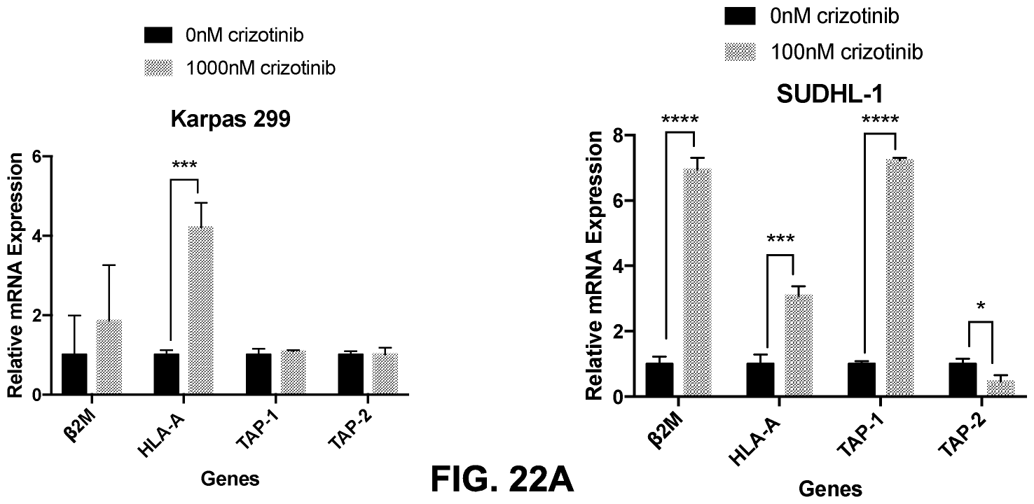

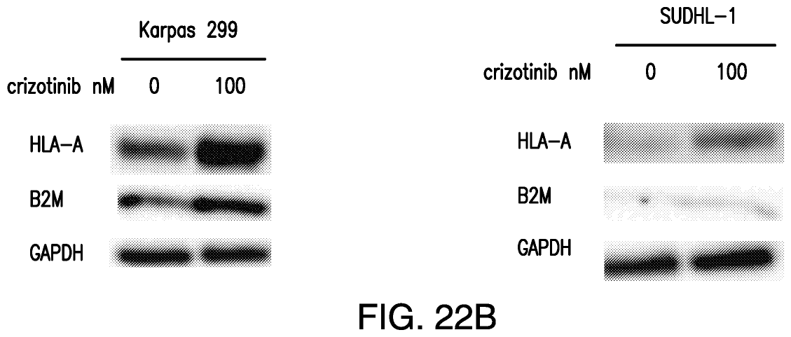

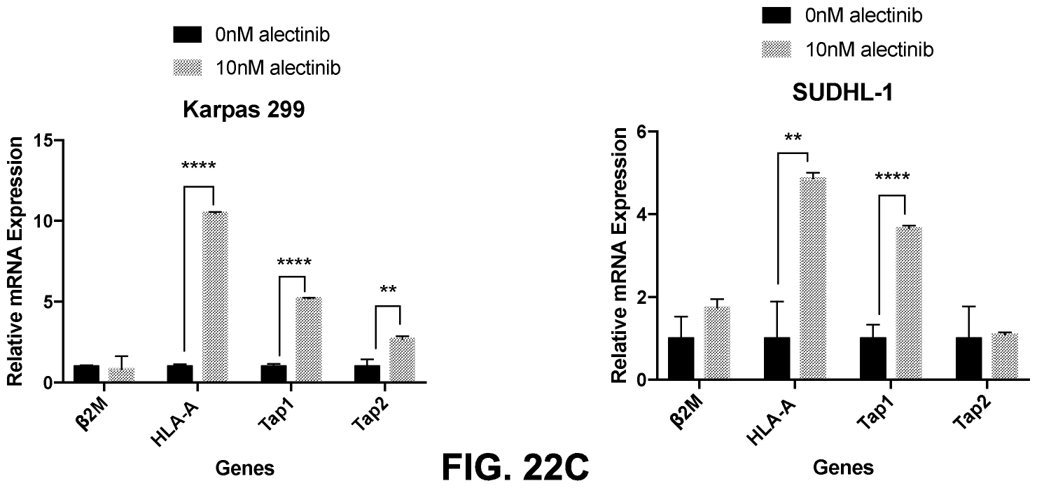

Nascent HLA molecules reside in the endoplasmic reticulum until they associate with beta-2-microglobulin, after which TAP1 and TAP2 transport the proteasome-cleaved peptides into the ER and antigenic peptides are loaded onto the complex. This complex is shuttled to the cell surface and later recycled back through endosomes (Blum et al., 2013, Annu Rev Immunol 31:443-473). Therefore, the increase in cell surface HLA could have been the result of increased transcription or translation, increased stabilization by peptide loading and beta-2-microglobulin association, or reduced degradation. The increase in surface HLA seen from RTK inhibition resulted from an increase in protein levels of HLA, indicating an effect on total molecule numbers (FIGS. 21A-21D, 22A-22D and 34). The drugs also caused variable increases in transcript levels of HLA and other proteins involved in antigen processing machinery, though in general there was an increase in either HLA and/or TAP1, TAP2, or beta-2-microglobulin (FIGS. 21A-21D, 22A-22D and 34). The magnitude of change seen in each of the proteins varied based on the cell line and inhibitor used and was not always coordinated across all proteins, suggesting other differences among these cell lines in the control of these processes. The increase in TAP1 and TAP2 and beta-2-microglobulin, indicated the potential for more peptide loading in the ER and more stabilization of the cell surface HLA.

6.3.4.4. The Role of the JAK/STAT Pathway in HLA Expression

STAT1 is a primary regulator of HLA and other antigen presentation machinery proteins (Gobin et al., 1999, J Immunol 163(3):1428-1434; Min et al., 1996, J Immunol 156(9):3174-3183). STAT1 increases HLA by activating transcription of IRF1, a transcription factor that binds to ISRE and activates transcription of HLA-A, HLA-B, HLA-C, and HLA-F (Gobin et al., 1999, J Immunol 163(3):1428-1434; Girdlestone et al., 2006, Proc Natl Acad Sci 90(24):11568-11572). This STAT1-mediated increase in surface HLA is also reported with EGFR inhibitors (Srivastava et al., 2015, Cancer Immunol Res 3(8):936-945; Pollack et al., 2011, Clin Cancer Res 17(13):4400-4413). EGFR is a receptor tyrosine kinase that feeds into the MAPK pathway, and when inhibited, leads to decreases in pERK (Marzi et al., 2016, Br J Cancer 115(10):1223-1233; Piotrowska et al., 2018, Cancer Discov 8(12):1529-1539). STAT1 is driving the changes in HLA mRNA, protein and cell surface expression after MAPK pathway inhibition (Brea et al., 2016, Cancer Immunol Res 4(11):936-947; Gobin et al., 1999, J Immunol 163(3):1428-1434; Min et al., 1996, J Immunol 156(9):3174-3183). Activated MAPK associated kinases (including ERK1 and ALK) directly reduce activated pSTAT1, which promotes proteasomal degradation of pSTAT1 via PIAS1 (Wu et al., 2015, Blood 126(3):336-345; Liu et al., 1998, Proc Natl Acad Sci 95(18):10626-10631; Zhang et al., 2018, BMC Cancer 18(1):613; Vanhatupa et al., 2008, Biochem J 409(1):179-185).

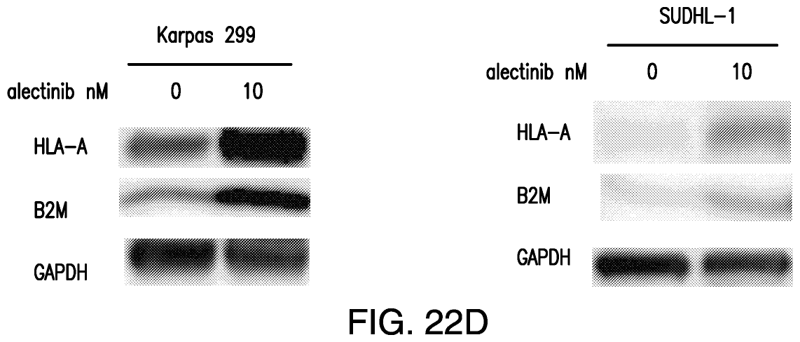

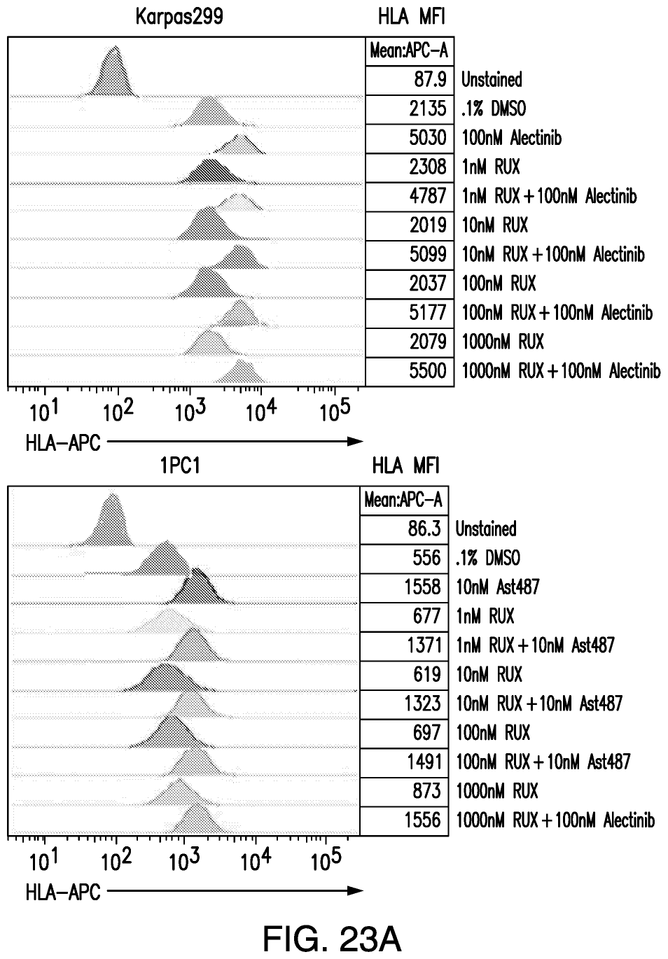

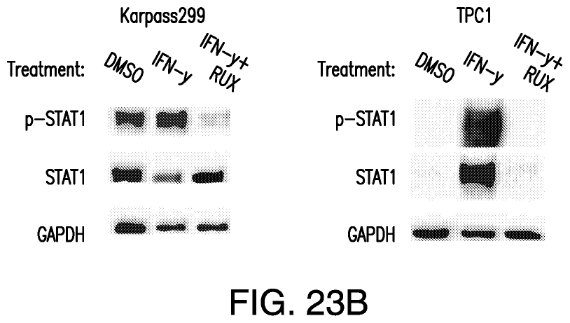

JAK is a primary activator of STAT1 when stimulated with IFN gamma (Gobin et al., 1999, J Immunol 163(3):1428-1434; Min et al., 1996, J Immunol 156(9):3174-3183); however, specific inhibition of JAK with ruxolitinib had no effect on the upregulation of HLA expression in either ALK mutated Karpas 299 cells or RET mutated TPC1 cells after specific inhibition of their respective oncogenic kinases (FIG. 23A). As a control, ruxolitinib blocked IFNγ-mediated upregulation of HLA at these doses (FIG. 23B).

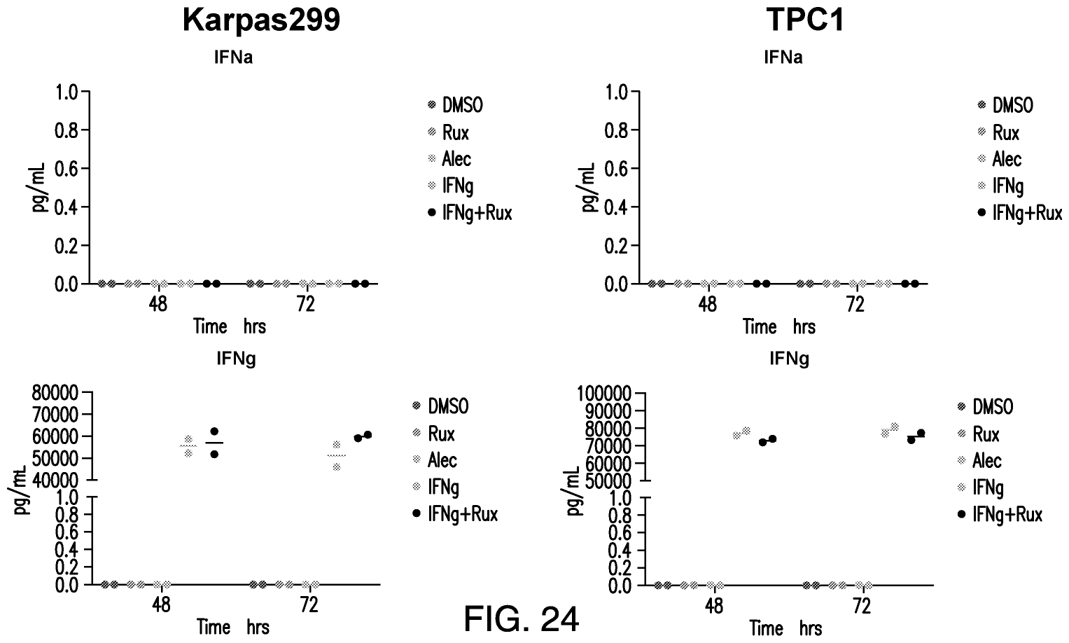

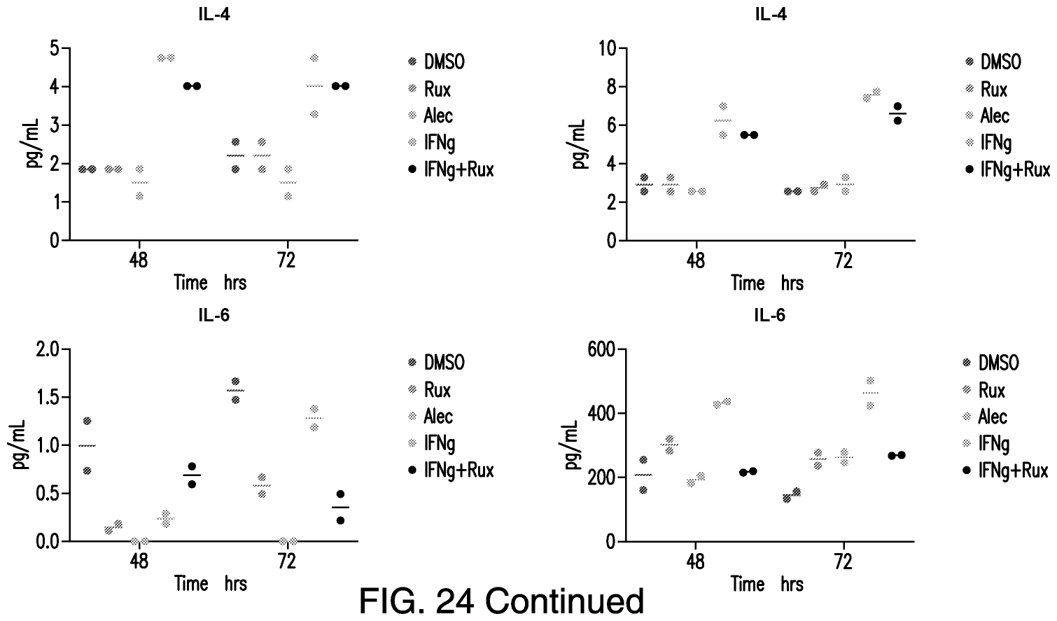

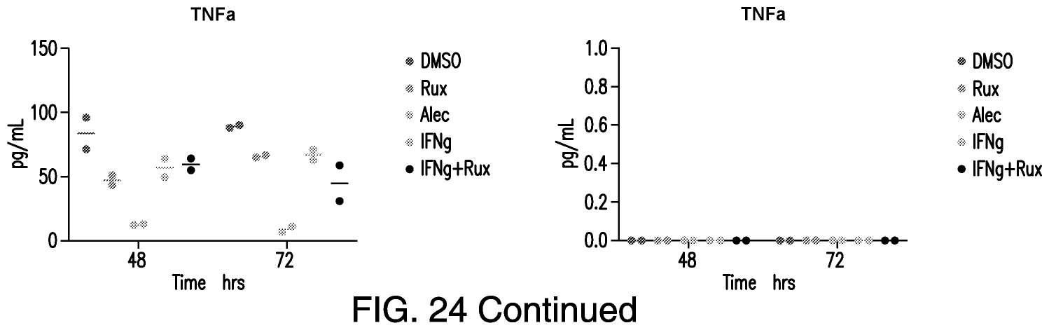

This study also examined if there was evidence of cytokine secretion by the cancer cells in response to ALK or RET inhibition that might account for indirect activation of the more traditional JAK/STAT pathway (FIG. 24). Alectinib inhibition in Karpas 299 lymphoma had no effect on IFNα, IFNγ, IL4, and reduced IL6 and TNFα secretion (FIG. 24). AST487 inhibition in TPC1 thyroid cells had no effect on IFNα, IFNγ, IL4, IL6 or TNFα secretion (FIG. 24). Therefore, it was unlikely that these inhibitors acted to upregulate the JAK/STAT pathway either directly, as shown above, or indirectly, by increased cytokine release. As a positive control, IFNγ increased both IL4 and IL6 in these cells, which was reduced by ruxolitinib (FIG. 24). Therefore, the dominant mechanism for the activity seen here in response to ALK or RET inhibition appeared to be loss of the direct reduction in pSTAT1 by the MAPK associated enzymes.

6.3.4.5. Increase of HLA Expression after MAPK Inhibition was Produced In Vivo

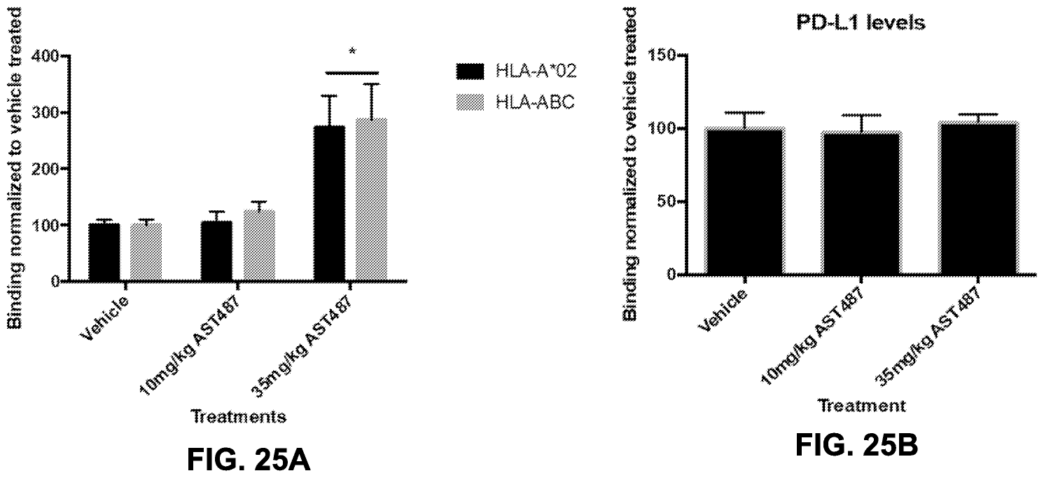

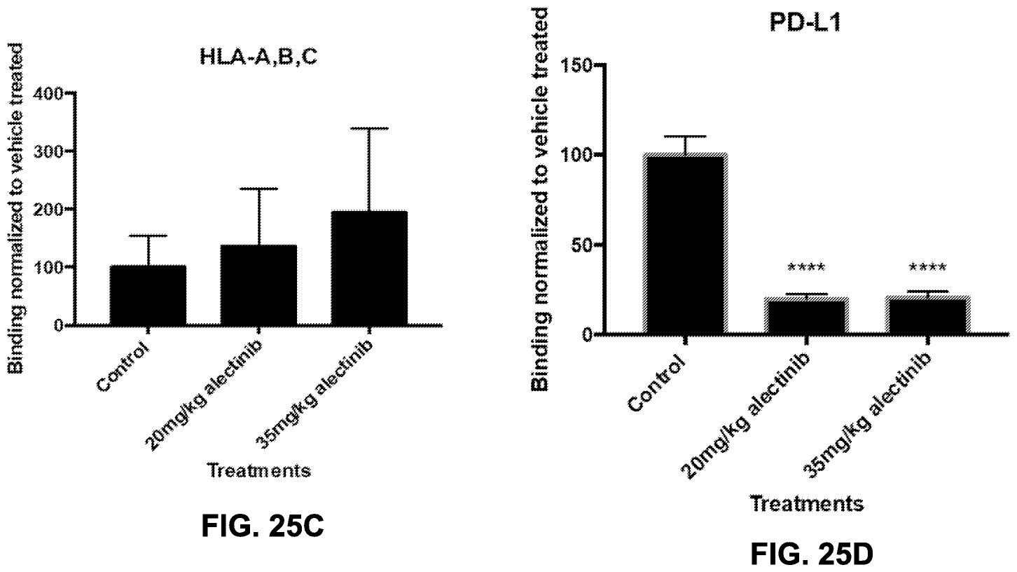

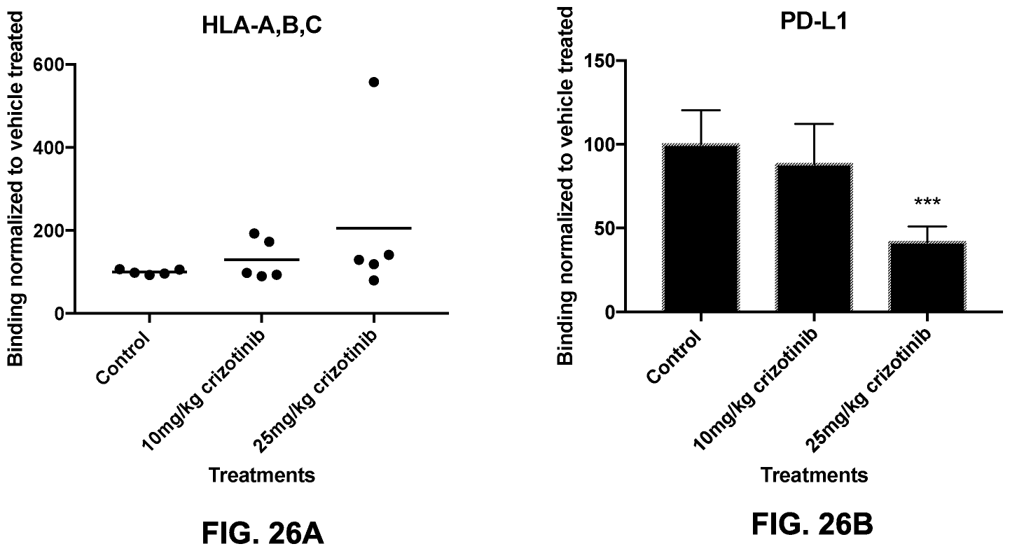

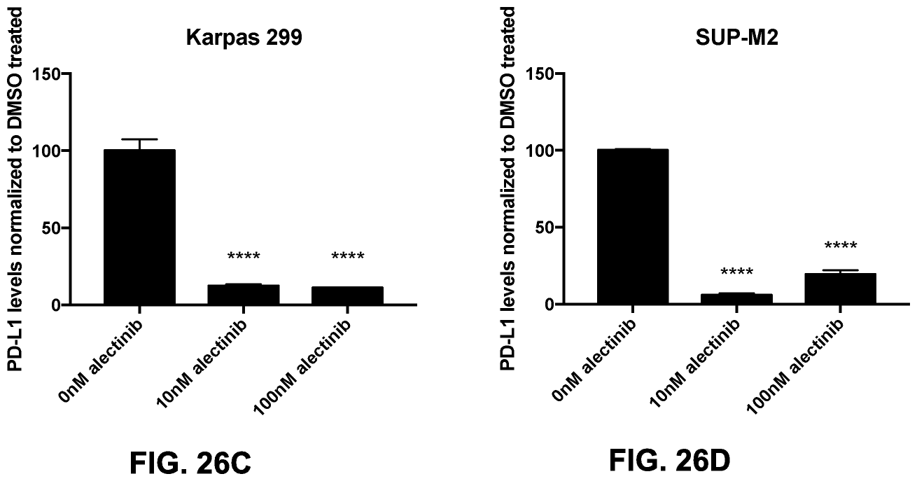

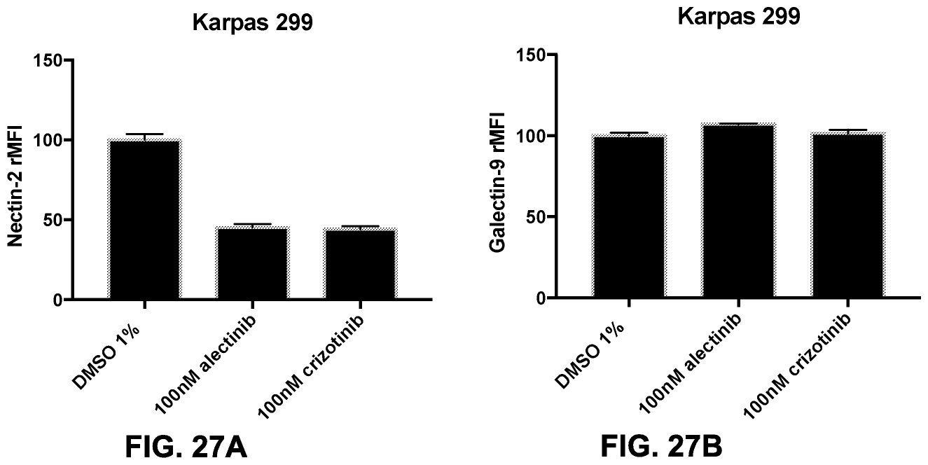

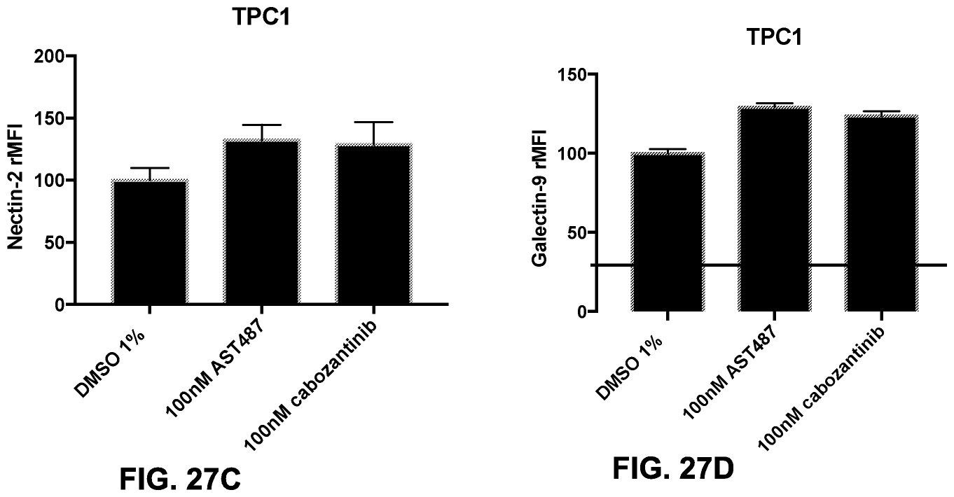

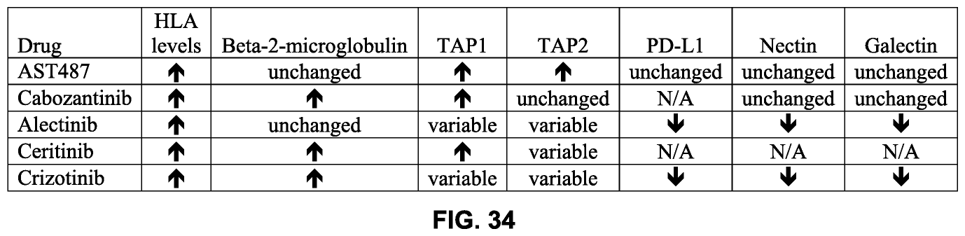

To determine if the RET and ALK inhibitors produced similar effects in live animals, mice bearing TPC1 and Karpas 299 tumors were treated with RET inhibitors. The highly immunodeficient NRG (NOD-Rag1^{null} IL2rg^{null}) mice were subcutaneously injected with luciferase tagged TPC1 cells in their flank. When the tumors were palpable, mice were given vehicle or AST 487 through once daily oral gavage for 7 days. Afterwards, cells were harvested immediately and stained with antibodies against HLA-A*02 and HLA-ABC. Dose-related increases in all HLA class I levels were seen with AST487 treatment, indicating that HLA also can be upregulated in vivo by RET inhibition (FIG. 25A). Moreover, PD-L1 levels were measured and no increase in PD-L1 was seen (FIGS. 25B and 34). These data suggest a potential use for RET inhibition in combination with T cell-based therapies or checkpoint blockade inhibition. NSG mice were also injected with Karpas 299 cells and treated with vehicle or ALK inhibitors, crizotinib or alectinib. Levels of HLA increased modestly in a dose-dependent manner for both drugs, though not all mice responded (FIGS. 25C and 26A). PD-L1 decreased with increasing doses of crizotinib and alectinib (FIGS. 25D, 26B and 34). Treatment with alectinib dropped PD-L1 by approximately 75% (FIG. 25D). The dramatic decreases in PD-L1 were seen in vitro as well (FIGS. 26C and 26D). ALK inhibition was able to decrease levels of nectin-2, another checkpoint ligand that binds to TIGIT (FIGS. 27A and 34). However, ALK inhibition did not affect all checkpoint ligands, as levels of galectin-9, the ligand of TIM-3, stayed constant (FIGS. 27B and 34). RET inhibition did not alter either of these ligands (FIGS. 27C, 27D and 34). Altogether, these effects could have a profound impact on using RET and ALK inhibitors with therapies that rely on T cells.

6.3.4.6. RET Inhibition Altered the Surface Immunopeptidome and Increased Peptide Presentation

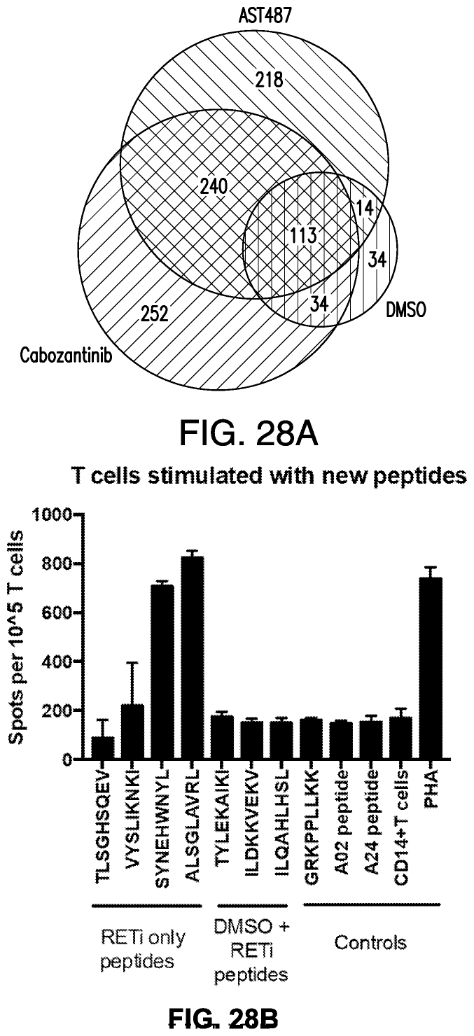

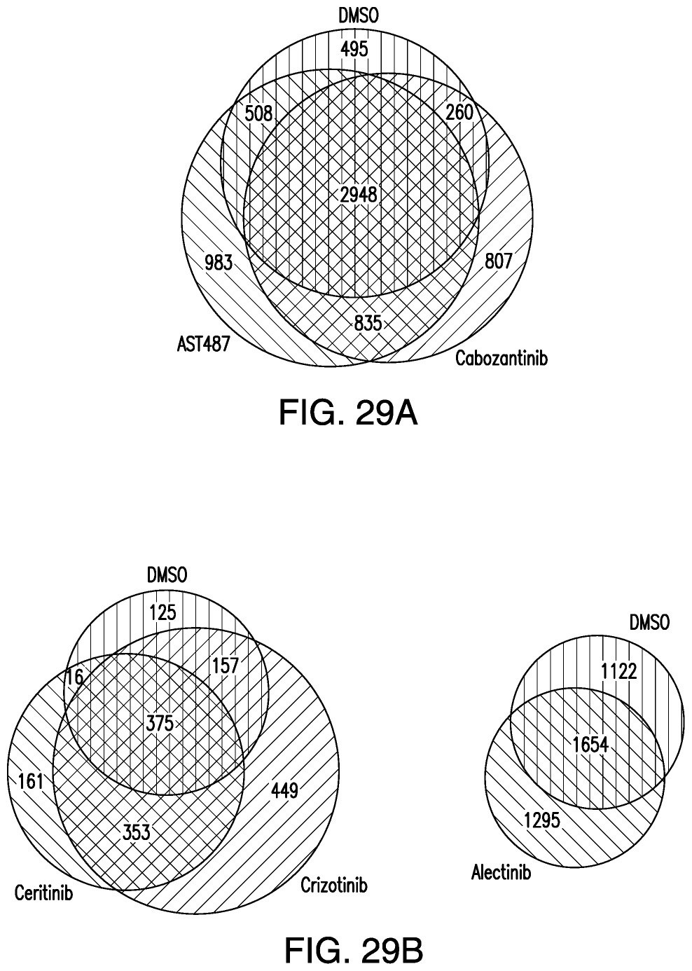

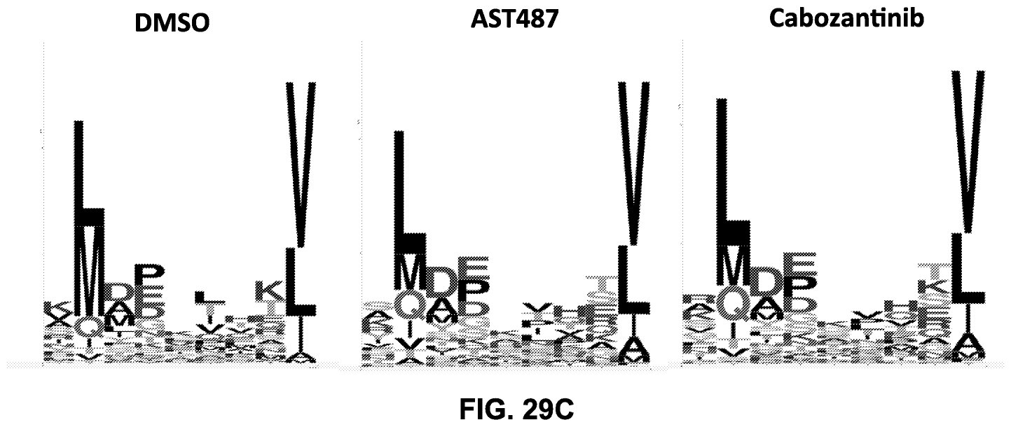

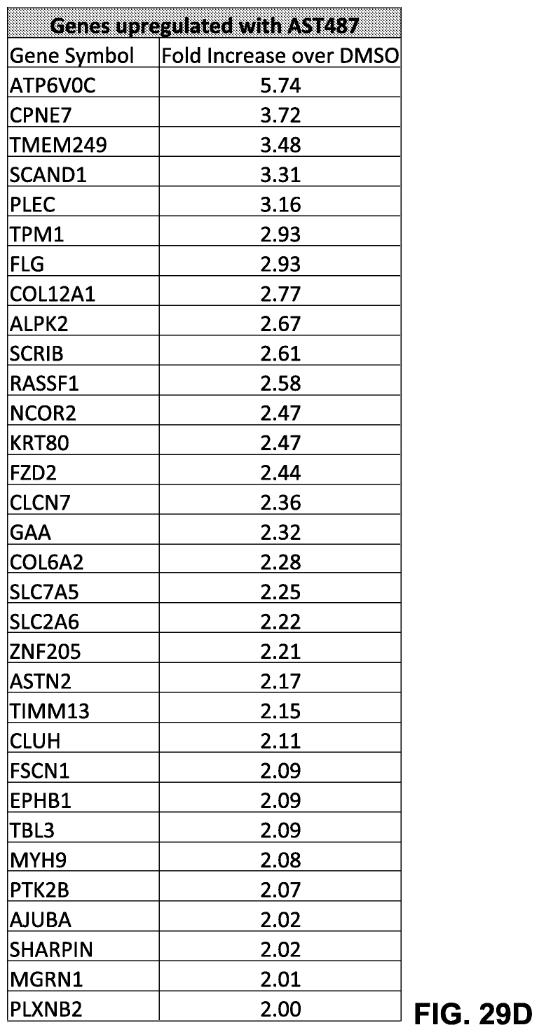

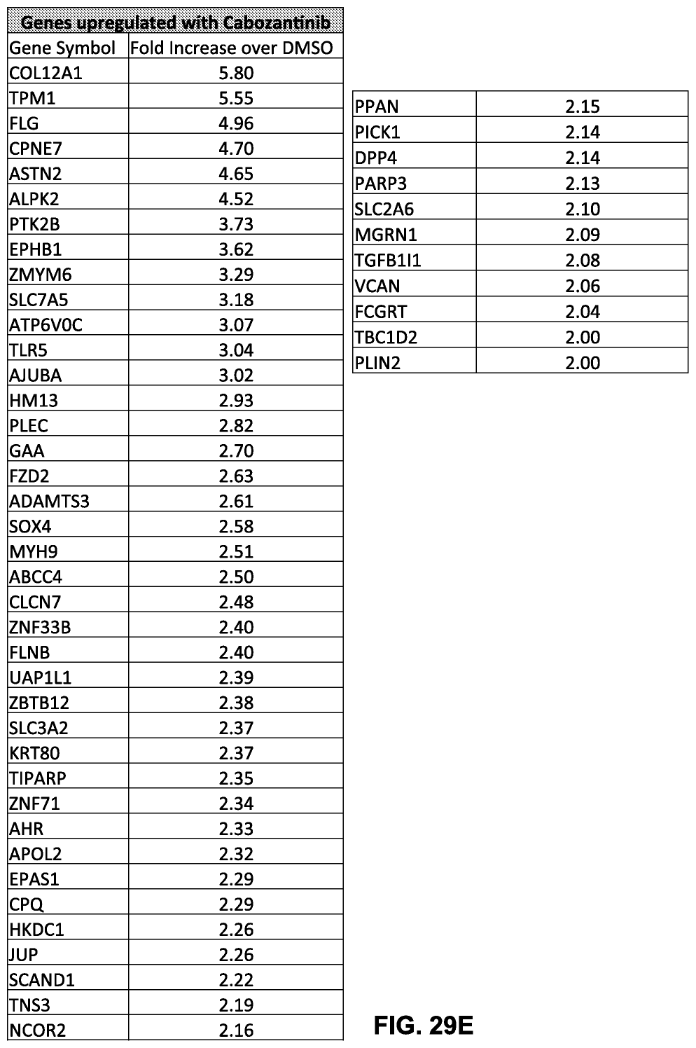

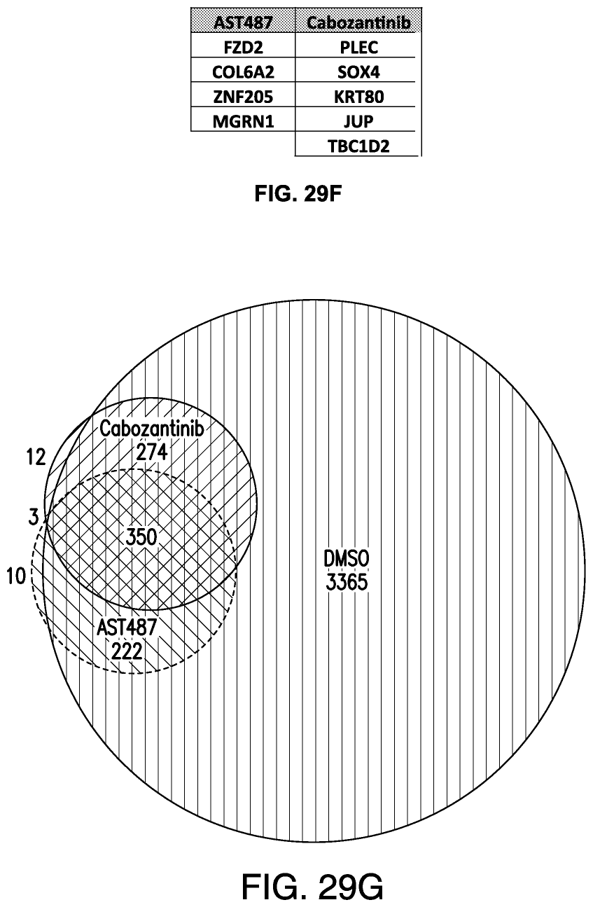

The increases in cell surface HLA and antigen presentation machinery after drug treatment suggested it was possible that the peptide repertoire would also be altered with RTK inhibition. Such changes could provide a second rationale for the use of these inhibitors as immune adjuvants or immunotherapies by enabling presentation of novel tumor associated antigens or neoantigens to be recognized by T cell based therapies (Schumacher and Schreiber, 2015, Science 348(6230):69-74; Yarchoan et al., 2017, Nature Reviews Cancer 17(4):209-222). Cells were treated with AST487 or cabozantinib for 3 days and then the peptides presented by HLA class I were analyzed by mass spectrometry. The peptides acquired from 3 independent experiments were profiled, comparing untreated cells with drug treated cells, and the number and sequences of unique peptides that were complexed with cell surface HLA molecules were determined. Peptides detected in all 3 runs were analyzed preferentially (FIG. 28A), as these HLA ligands represented the most robust group. RET inhibitor treated groups yielded 3-fold higher amounts of unique HLA ligands compared to the DMSO group: 639 for Cabozantinib, 585 for AST487 and 195 for the DMSO vehicle control group (FIG. 28A). Half of the peptides seen only in the treated subgroups (240 unique ligands) were shared between the two treatment groups (FIG. 28A). 34 HLA presented peptides from the DMSO group could not be identified anymore after drug treatment (FIG. 28A). Overall, this analysis showed a large increase in detectable HLA ligands for the TKI treated subgroups. The changes in unique HLA ligands were also analyzed when the new peptide was found in only one or two of the three biological mass spectrometry replicates performed; in this case, the number of new peptides increased 5 fold after drug treatment (FIG. 29A). A similar shift in new antigens and peptide repertoire was seen with ALK inhibition by the three different ALK inhibitors when treating Karpas 299 cells (FIG. 29B).

The appearance of hundreds of new cell surface peptides complexed with HLA class I that are not present before drug treatment increased the chance of presentation of immunogenic peptides with this group. The immune response of HLA-A*02:01 positive healthy donors to a small sample of the newly presented HLA ligands was tested. Through an IFN-gamma ELISpot assay, several of the peptides arising after drug treatment were shown to be immunogenic. Human T cells were stimulated against a sample pool of four of the HLA ligands (TLSGHSQEV (SEQ ID NO:2), VYSLIKNKI (SEQ ID NO:3), SYNEHWNYL (SEQ ID NO:4), ALSGLAVRL (SEQ ID NO:5)). Two of the four new antigens were able to elicit T cell mediated IFN gamma response to autologous CD14⁺ cells presenting those corresponding peptides (FIG. 28B). No response of these cells was seen against several control peptides that were found before drug treatment on TPC1 cells (TYLEKAIKI (SEQ ID NO:6), ILDKKVEKV (SEQ ID NO:7), ILQAHLHSL (SEQ ID NO:8)) or to an irrelevant peptide (GRKPPLLKK (SEQ ID NO:9)) (FIG. 28B).

To further understand the possible biochemical mechanisms behind the repertoire shift, the motifs of the peptides found in each treatment group were first examined to determine if drug treatment was altering protein cleavage and processing (FIG. 29C). Processing of the peptides did not appear to be altered substantially, as the frequency of amino acids in each position was similar before and after treatment, thus not accounting for these large changes in the repertoire (FIG. 29C). Next, RNA-seq was performed on the cells treated with RET inhibitors to determine if the drugs were altering protein expression and thus the ligandome. The protein derivation of the peptides in the ligandome was also analyzed in comparison to the upregulated proteins in each cell group (FIGS. 29D-29G). Network analysis of new peptides after RET inhibition showed enrichment in the cell cycle arrest and negative regulation of the cell cycle pathways (FIGS. 29D and 29E). Among the new peptides in the RET inhibitor treated cells, 2 out of the 16 known TEIPPs with spontaneous immune responses in healthy donors presented on HLA-A*02:01 were detected (Table 1) (Marijt et al., 2018, J Exp Med 215(9):2325-2337). Previously, TEIPPs have been described in cells that lack TAP or are low in HLA surface expression (Marijt et al., 2018, J Exp Med 215(9):2325-2337; Kiessling, 2016, Journal of Clinical Investigation 126(2):480-482; Lampen et al., 2010, J Immunol 185(11):6508-6517; Komov et al., 2018, Proteomics 18(12):e1700248). This study instead showed that in cells that have increased levels of HLA and TAP proteins, TEIPPs were presented, suggesting an alternate mechanism for their appearance.

TABLE 1
HLA-A*02:01 TEIPP peptides found after RET inhibitor treatment.
(The 16 known TEIPP peptides reported for HLA-A*02 and the drug
treatment groups in which those TEIPP peptides were found. Number
indicates runs TEIPP peptide were found in (n = 3)).

| TIEPP sequence | DMSO | AST487 | Cabozantinib |
|---|---|---|---|
| ALFSFVTAL (SEQ ID NO: 11) | 0 | 0 | 0 |
| FLGPWPAAS (SEQ ID NO: 12) | 0 | 1 | 1 |
| FLSELQYYL (SEQ ID NO: 13) | 0 | 0 | 0 |
| FLYPFLSHL (SEQ ID NO: 14) | 0 | 0 | 0 |
| ILEYLTAEV (SEQ ID NO: 15) | 0 | 0 | 0 |
| LLALAAGLAV (SEQ ID NO: 16) | 0 | 0 | 0 |
| LLLDVPTAAV (SEQ ID NO: 17) | 0 | 0 | 1 |
| LLLSAEPVPA (SEQ ID NO: 18) | 0 | 0 | 0 |
| LLWGRQLFA (SEQ ID NO: 19) | 0 | 0 | 0 |
| LSEKLERI (SEQ ID NO: 20) | 0 | 0 | 0 |
| LTLLGTLWGA (SEQ ID NO: 21) | 0 | 0 | 0 |
| SVLWLGALGL (SEQ ID NO: 22) | 0 | 0 | 0 |
| TLLGASLPA (SEQ ID NO: 23) | 0 | 0 | 0 |
| VIIKPLVWV (SEQ ID NO: 24) | 0 | 0 | 0 |
| VLAVFIKAV (SEQ ID NO: 25) | 0 | 0 | 0 |
| VLLDHLSLA (SEQ ID NO: 26) | 0 | 0 | 0 |

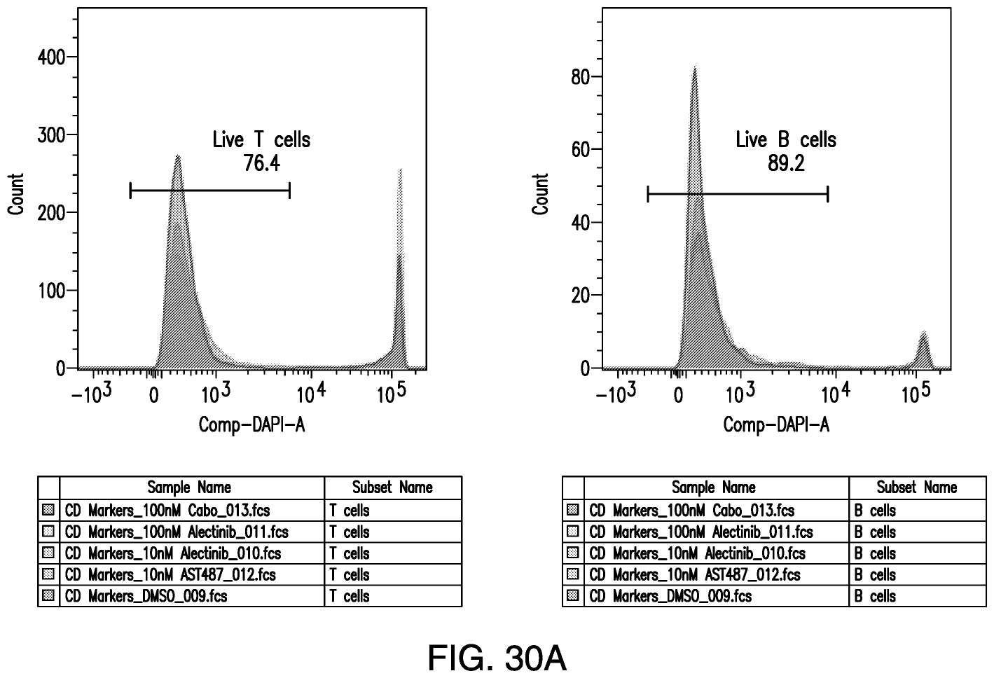

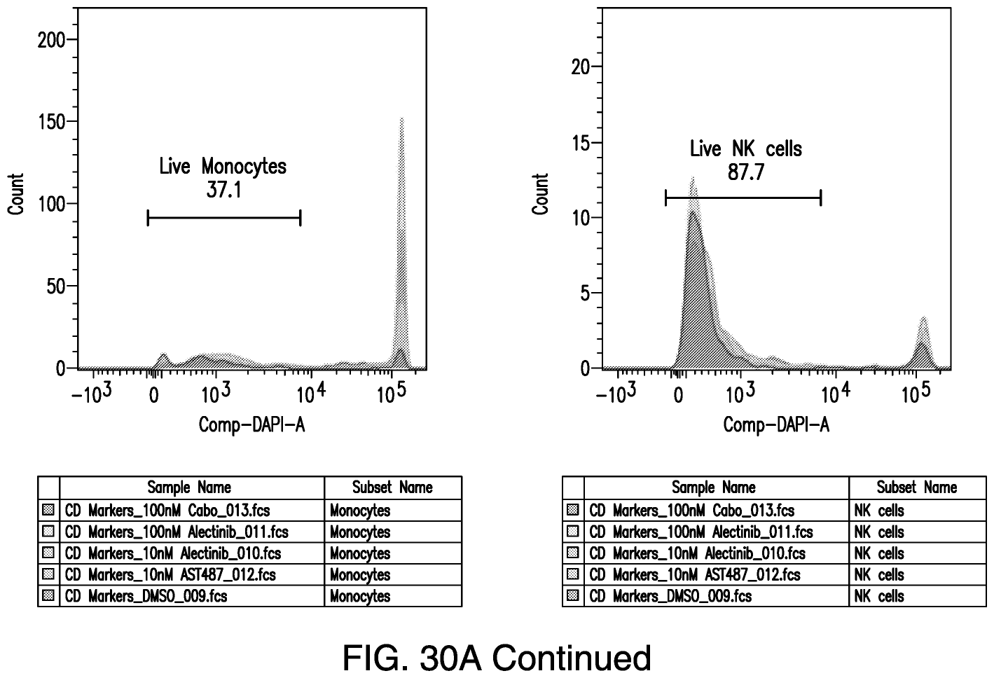

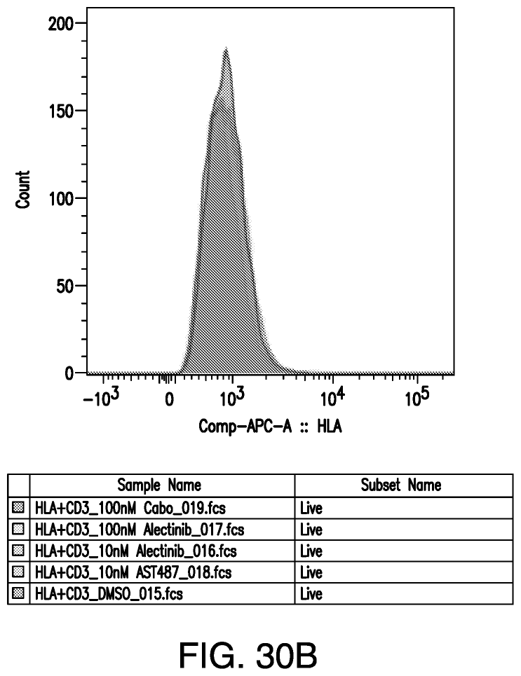

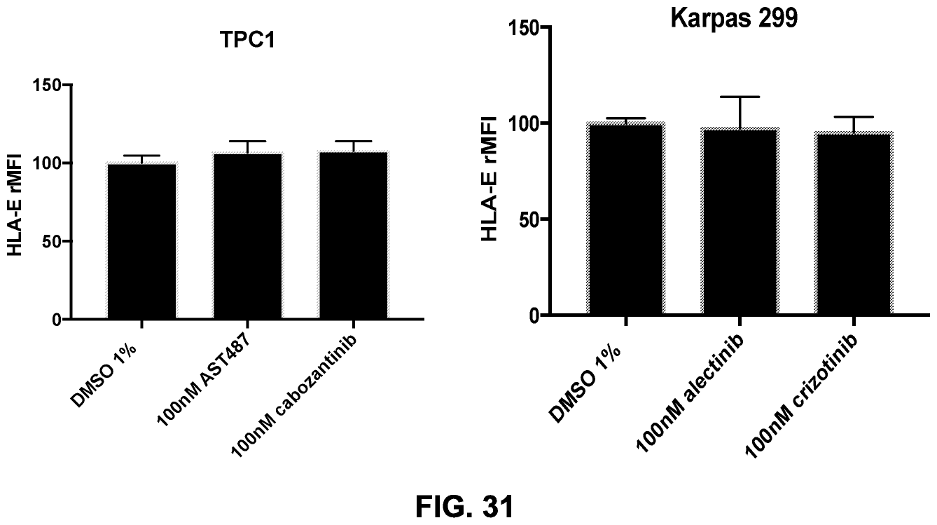

PBMC viability and HLA levels were not affected by the inhibitors, suggesting specificity of the drugs for cells with these altered pathways (FIGS. 30A and 30B). In addition, only the canonical HLA molecules were affected, while noncanonical molecules like HLA-E were unchanged (FIG. 31).

6.3.4.7. Unmasked Antigens after RET Inhibition Enhanced Cellular Cytotoxicity Against HLA Complexes

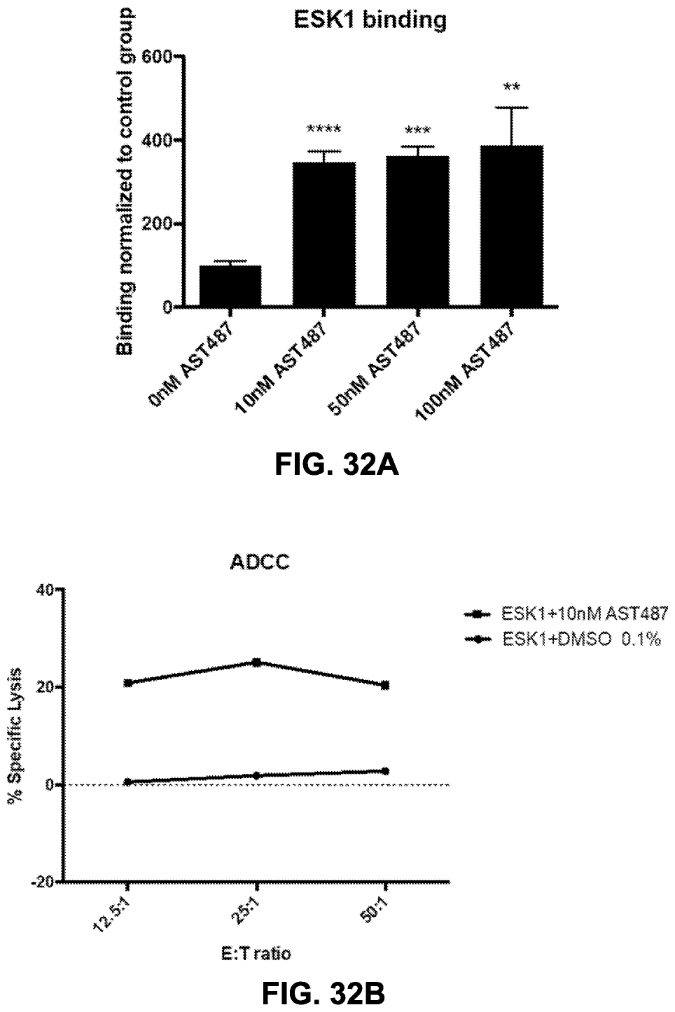

TCR mimic monoclonal antibodies (TCRm) recognize peptide/HLA complex epitopes in a manner similar to that of a TCR, but have the advantageous pharmacological properties of an antibody (Dubrovsky et al., 2014, Blood 123(21):3296-3304). ESK1 is a TCR mimic antibody that reacts with the RMFPNAPYL (SEQ ID NO:1) peptide derived from WT1 as well as several other peptides with similar sequences, when complexed with HLA-A*02:01 (Veomett et al., 2014, Clin Cancer Res 20(15):4036-4046). Although, ESK1 bound minimally to naïve TPC1 cells, increased ESK1 binding was seen following RET inhibition (FIG. 32A). Analysis of the mass spectrometry data show that TPC1 cells do not present known epitopes that could be bound by ESK1 before treatment; however, after RET inhibition, the off target peptide (RMFPGEVAL (SEQ ID NO:10)) is present and allows binding of ESK1 (Gejman et al., 2019, bioRxiv, doi: doi.org/10.1101/267047). Hence, ESK1 was used as a tool to show that the increased HLA expression and presentation of new peptides following RET inhibition resulted in improved antibody-dependent cellular cytotoxicity (ADCC) activity when TPC1 cells were pre-incubated with the RET inhibitor, AST487 (FIG. 32B). Thus, the ability to unmask new antigens has the potential to enhance TCR-based recognition and increase T-cell mediated lysis of target cells.

6.3.5. Discussion

With the plethora of effective T cell-based therapies for cancer, the ability to safely increase HLA levels could have a profound impact across a wide variety of treatments. The minimal, or absence of, ALK and RET expression on normal cells make these RTKs appealing targets for selective HLA upregulation on cancer cells that express their activated forms, with little risk for side effects. Whereas it has previously been shown that MEK inhibitors also upregulated HLA, these drugs also adversely affect T cell function (Brea et al., 2016, Cancer Immunol Res 4(11):936-947; Dushyanthen et al., 2017, Nat Commun 8(1):606; Liu et al., 2015, Clin Cancer Res 21(7):1639-1651; Mimura et al., 2013, J Immunol 191(12):6261-6272). ALK and RET inhibitors act upstream of other pathways and if more than one pathway affecting HLA is inhibited, this could lead to an additive effect on HLA upregulation.

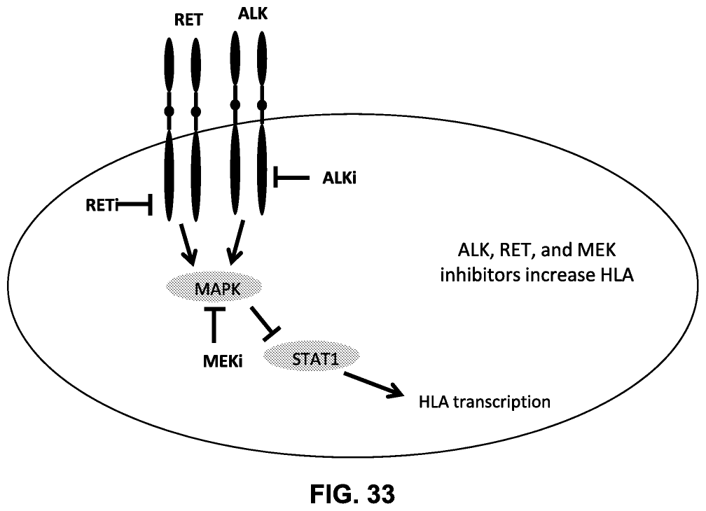

This Example has shown consistently, with multiple drugs and RNAi, using multiple cell lines, that inhibition of ALK and RET led to substantial increases in the surface levels of HLA-A,B,C. FIG. 33 summarizes a proposed model on the signaling pathway for HLA upregulation. Antigen processing machinery transcript and proteins in cells that contain the respective target mutant oncogenes also increased with inhibition. These large changes in HLA complex quantities in cancer cells have wide implications for T cell immunosurveillance. The amount of HLA complexes, but not the amount of peptides present in the ER is the limiting factor in HLA ligand presentation (Komov et al., 2018, Proteomics 18(12):e1700248). Increases in HLA complex capacities, as were demonstrated in this study, not only provides the opportunity to display more of the same peptides, but also rarely presented HLA ligands. This hypothesis was confirmed by multiple mass spectrometry experiments with TKI inhibition, resulting in large changes in the quantity and quality of the immunopeptidome, with potentially hundreds of new epitopes displayed on the cell surface. As escape from the immune system is a hallmark of cancer survival and progression, these data provide another mechanism by which oncogenesis is promoted, by downregulating antigen presentation during the oncogenic process (Hanahan and Weinberg, 2011, Cell 144(5):646-674). Thus, ALK and RET inhibitors could be used in combination with T cell immunotherapies by making cancer cells more susceptible to T recognition (French, 2013, Thyroid 23(5):529-542).

The appearance of the numerous new peptide epitopes may be a consequence of several mechanisms: 1. increased detection rate in mass spectrometry experiments due to presentation of the same peptides in higher numbers, 2. altered gene expression due to pathway inhibition, or 3. altered protein processing and new cleavage patterns of new and existing proteins. First, the several fold increases in cell surface HLA molecules (perhaps hundreds of thousands of additional HLA molecules per cell), which in concert with the increased antigen processing machinery, could lead to large increases in total presented peptides and thus the increased sensitivity of T cells to recognize the rarer epitopes. It is possible that the increase in epitopes detected by mass spectrometry may be in part due to the increase in absolute number of the same peptides that were already present, but now presented at higher frequencies due to increased HLA expression. However, the potential for increased cell surface HLA expression to bias the sensitivity of detection of rare peptides does not seem to be a sufficient explanation for their detection, since results of the overlap of the ligandome from three individual experiments showed not only a disappearance of many HLA ligands found in the control group, which otherwise should still be detected, but also identified two distinct new groups after two RET inhibitor treatments. There was only 30% overlap of HLA ligands appearing after either TKI treatment; it is hypothesized that this proportion would have to be much higher to argue that their appearance is only mediated exclusively by increase of detection rates after upregulating HLA levels. Thus, in addition to the increase in HLA expression, the ALK and RET inhibitors could also alter the cell's protein repertoire independent of the effects on antigen presentation pathway throughput, thus providing potential new antigens. The inhibited ALK and RET kinases are upstream of multiple signaling pathways that control expression of multiple target genes. This could lead to the appearance of the new peptides found in the drug treated groups, which could potentially include tumor-associated antigens.

The generation of new peptides could have resulted from altered proteasomal cleavage patterns of proteins that normally need to be degraded for HLA presentation, as has been seen with interferon gamma (Chang et al., 2017, J Clin Invest 127(7):2705-2718; Gravett et al., 2018, Oncoimmunology 7(6):e1438107). However, motif analysis of the A*02 9-mer peptides in each treatment group yielded mostly identical frequencies of amino acids over all positions, arguing against the changes in proteasomal cleavage as a major explanation for the detection of many new HLA ligands after TKI treatment.

Analysis of RNA-Seq data showed the 32 genes were upregulated at least two-fold in the AST487 treated cells and 50 genes in the cabozantinib treated cells. However, altered gene expression as an effect of TKI treatment was not sufficient to fully explain changes in the HLA ligand repertoire since only a very small fraction (four for AST487 and five for Cabozantinib, 1.2% of total new peptides) of newly identified HLA ligands in the treatment groups showed at least a 2-fold increase in mRNA levels. Overall, this indicates that most of the new peptides might have been detected because of the overexpression of HLA in combination with MAPK pathway alterations after TKI treatment. This notion was further supported in the comparison between RET inhibitor altered peptides present in all three experiments and control peptides present in at least one of the experiments (FIG. 29G). Almost all of the most robustly expressed peptides that were presented in all three experiments after RET inhibition, were found at least once in the control cells as well (3365 total peptides). Despite this overlap in presentation, there were more than two dozen peptides regularly revealed by the inhibitors that were never found presented in the control cells. Looking at the 2 RET inhibitor revealed peptides found in at least one run, the number of new peptides was broadened to 1818 and 1642 peptides, for the 2 drugs respectively, that were never present in the control treatment group. These data lead to the conclusion that the peptides displayed on the cancer cell at a given time are derived from a large pool (for instance, 195 control peptides were seen in all three runs, compared to the 3365 control peptides that were seen at least one time). RET inhibitors caused potential convergence and better detection of the peptides displayed. RET inhibition leads to new peptides with a minimum of 25 found in all three runs to a maximum of a few thousand peptides found at least once in the three experiments.

From the pool of new peptides, the presence of TEIPPs further expands the therapeutic potential of RET inhibition by presenting a set of known neoantigens that have detectable frequencies of CD8⁺ cognate T cells and to which CD8⁺ T cells have shown reactivity (Marijt et al., 2018, J Exp Med 215(9):2325-2337; Lampen et al., 2010, J Immunol 185(11):6508-6517). It is generally assumed that TEIPPs are found primarily in TAP deficient cells, in which peptides from the cytoplasm are limited, and there is excess HLA capacity. Here, this Example showed an alternative mechanism for TEIPP presentation, in which markedly increasing the total HLA carrying capacity led to presentation of these unusual TEIPP epitopes by changing the ratio of available HLA molecules to available peptides. Based on the RNA seq and motif data, there is probably no generation of new TEIPPs through transcription or cleavage. TEIPPs are not normally presented due to the large abundance of processed peptides with favorable binding characteristics from which the limited HLA molecules can choose. Instead, here, by increasing HLA abundance these normally unselected TEIPP peptides were allowed to be loaded into HLA (Komov et al., 2018, Proteomics 18(12):e1700248). These data again underlie the ability of kinase inhibition to shift the peptide repertoire to produce immunogenic peptides. Because a random sampling of the new peptides that appeared after RET inhibition were capable of stimulating an immune response in human T cells, it is hypothesized that the new peptide repertoire may enhance the immunogenic potential of cancer cells in the setting of inhibitor therapy.

In conclusion, this study identified a new strategy for upregulating the expression of HLA in ALK and RET mutated cancers in vitro and in vivo by using ALK and RET tyrosine kinase inhibitors. This increase in the expression of HLA in cancer cells can make those cells preferable targets for T-cell based immunotherapies. It was demonstrated that this increase in HLA binding capacities gave rise to a distinct new repertoire of HLA ligands, which were capable of eliciting CD8⁺ T cell-responses and can mediate ADCC through TCR mimic antibodies, as a surrogate for T cell killing. The detection of TEIPPs in this new repertoire gives new insights into the biology of these rare HLA ligands and expands the list of potential tumor-specific targets which are induced through RET and ALK inhibitor treatment (Marijt et al., 2018, J Exp Med 215(9):2325-2337; Lampen et al., 2010, J Immunol 185(11):6508-6517). When these effects are taken together, ALK and RET inhibitors provide a method to increase HLA expression in cancer cells and simultaneously unmask hundreds of treatment induced HLA ligands capable of inducing T cell responses. This opens up the potential for combinatorial therapies of ALK and RET inhibition and subsequent TCR-based immunotherapy.

### 7. INCORPORATION BY REFERENCE

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

What is claimed is:

1. A method of treating a cancer in a patient comprising: (i) administering to the patient an inhibitor of the activity of a kinase, and (ii) administering to the patient an immunotherapy that promotes an immune response against the cancer; wherein the kinase is ALK (anaplastic lymphoma kinase).

2. The method of claim 1, wherein the inhibitor is crizotinib, ceritinib, or alectinib.

3. A method of treating a cancer in a patient comprising: (i) administering to the patient an inhibitor of the activity of a kinase, and (ii) administering to the patient an immunotherapy that promotes an immune response against the cancer; wherein the kinase is ERBB2 (erb-b2 receptor tyrosine kinase 2).

4. The method of claim 3, wherein the inhibitor is trastuzumab or lapatinib.

5. The method of claim 1 or 3, wherein the inhibitor is a small molecule inhibitor.

6. The method of claim 1 or 3, wherein the inhibitor is an antibody or an antigen-binding fragment thereof that specifically binds to the kinase.

7. The method of claim 6, wherein the antibody is a monoclonal antibody.

8. The method of any of claims 1-7, wherein the inhibitor is administered in a subclinical amount.

9. The method of any of claims 1-8, wherein the immunotherapy is a vaccine.

10. The method of any of claims 1-8, wherein the immunotherapy is an immune checkpoint blockade.

11. The method of claim 10, wherein the immune checkpoint blockade is an antibody or an antigen-binding fragment thereof that specifically binds to and reduces the activity of an immune checkpoint protein.

12. The method of claim 11, wherein the antibody is a monoclonal antibody.

13. The method of any of claims 10-12, wherein the immune checkpoint blockade inhibits the activity of CTLA-4, PD-1, PD-L1, PD-L2, TIM-3, or LAG-3.

14. The method of any of claims 1-8, wherein the immunotherapy is an adoptive immunotherapy.

15. The method of claim 14, wherein the adoptive immunotherapy is an adoptive T cell therapy.

16. The method of claim 15, wherein the adoptive T cell therapy is TCR (T-Cell Receptor)-engineered T cells.

17. The method of claim 15, wherein the adoptive T cell therapy is CAR (Chimeric Antigen Receptor) T cells, wherein the antigen-binding domain of the CAR specifically binds to an antigen of the cancer.

18. The method of any of claims 1-8, wherein the immunotherapy is a TCR mimic antibody.

19. The method of any of claims 1-8, wherein the immunotherapy is a TCR based construct that encodes a soluble protein comprising the antigen recognition domain of a TCR.

20. The method of any of claims 1-8, wherein the immunotherapy is an interferon, an anti-CD47 antibody, a SIRP alpha antagonist, an HDAC inhibitor, a cytokine, a TLR (Toll-Like Receptor) agonist, or an epigenetic modulator that upregulates the expression of one or more MHCs (Major Histocompatibility Complexes) or upregulates antigen presentation.

21. The method of claim 20, wherein the immunotherapy is an epigenetic modulator that upregulates the expression of one or more MHCs or upregulates antigen presentation that is a hypomethylating agent.

22. The method of claim 21, wherein the immunotherapy is a hypomethylating agent that is azacytidine or decitabine.

23. The method of claim 20, wherein the immunotherapy is an interferon that is interferon alpha or interferon gamma.

24. The method of claim 20, wherein the immunotherapy is a cytokine that is IL2 (Interleukin-2), TNF (Tumor Necrosis Factor), interferon alpha or interferon gamma.

25. The method of claim 20, wherein the immunotherapy is a TLR agonist that is a dsDNA (double-stranded DNA) TLR agonist.

26. The method of claim 20, wherein the immunotherapy is a TLR agonist that is a dsRNA (double-stranded RNA) TLR agonist.

27. The method of claim 26, wherein the immunotherapy is a dsRNA TLR agonist that is polyinosinic-polycytidylic acid (poly(I:C)).

28. A method of generating a population of antigen-presenting cells for therapeutic administration to a patient having a cancer, comprising culturing antigen-presenting cells that are loaded with or genetically engineered to express one or more immunogenic peptides or proteins derived from one or more antigens of the cancer in the presence of an inhibitor of the activity of a kinase, wherein the kinase is ALK.

29. A method of treating a cancer in a patient comprising generating a population of antigen-presenting cells according to the method of claim 28, and administering to the patient the population of antigen-presenting cells.

30. The method of claim 28 or 29, wherein the inhibitor is crizotinib, ceritinib, or alectinib.

31. A method of generating a population of antigen-presenting cells for therapeutic administration to a patient having a cancer, comprising culturing antigen-presenting cells that are loaded with or genetically engineered to express one or more immunogenic peptides or proteins derived from one or more antigens of the cancer in the presence of an inhibitor of the activity of a kinase, wherein the kinase is ERBB2.

32. A method of treating a cancer in a patient comprising generating a population of antigen-presenting cells according to the method of claim 31, and administering to the patient the population of antigen-presenting cells.

33. The method of claim 31 or 32, wherein the inhibitor is trastuzumab or lapatinib.

34. The method of any of claims 28-29 and 31-32, wherein the inhibitor is a small molecule inhibitor.

35. The method of any of claims 28-29 and 31-32, wherein the inhibitor is an antibody or an antigen-binding fragment thereof that specifically binds to the kinase.

36. The method of claim 35, wherein the antibody is a monoclonal antibody.

37. The method of any of claims 1-36, wherein the cancer is breast cancer, lung cancer, ovary cancer, stomach cancer, pancreatic cancer, larynx cancer, esophageal cancer, testes cancer, liver cancer, parotid cancer, biliary tract cancer, colon cancer, rectum cancer, cervix cancer, uterus cancer, endometrium cancer, renal cancer, bladder cancer, prostate cancer, thyroid cancer, melanoma, or non-small cell lung cancer.

38. The method of any of claims 1-36, wherein the cancer is lung cancer, thyroid cancer, or melanoma.

39. A method of treating an autoimmune disease in a patient comprising: (i) administering to the patient an activator of the activity of a kinase, and optionally (ii) administering to the patient an immunosuppressive therapy that suppresses the immune response associated with the autoimmune disease; wherein the kinase is ALK.

40. A method of treating an autoimmune disease in a patient comprising: (i) administering to the patient an activator of the activity of a kinase, and optionally (ii) administering to the patient an immunosuppressive therapy that suppresses the immune response associated with the autoimmune disease; wherein the kinase is ERBB2.

41. The method of claim 39 or 40, wherein the autoimmune disease is multiple sclerosis, type 1 diabetes, ankylosing spondylitis, or Hashimoto's thyroiditis.

42. A method of treating graft-versus-host disease (GvHD) in a patient comprising: (i) administering to the patient an activator of the activity of a kinase, and optionally (ii) administering to the patient an immunosuppressive therapy that suppresses the immune response associated with the GvHD; wherein the kinase is ALK.

43. A method of treating GvHD in a patient comprising: (i) administering to the patient an activator of the activity of a kinase, and optionally (ii) administering to the patient an immunosuppressive therapy that suppresses the immune response associated with the GvHD; wherein the kinase is ERBB2.

44. The method of claim 42 or 43, wherein the GvHD is an acute GvHD.

45. The method of claim 42 or 43, wherein the GvHD is a chronic GvHD.

46. A method of reducing the risk of solid organ transplant rejection in a patient comprising:
(i) administering to the patient an activator of the activity of a kinase, and optionally (ii) administering to the patient an immunosuppressive therapy that suppresses the immune response against the solid organ transplant; wherein the kinase is ALK.

47. A method of reducing the risk of solid organ transplant rejection in a patient comprising:
(i) administering to the patient an activator of the activity of a kinase, and optionally (ii) administering to the patient an immunosuppressive therapy that suppresses the immune response against the solid organ transplant; wherein the kinase is ERBB2.

48. The method of any of claims 39-47, wherein the activator is administered in a subclinical amount.

49. The method of any of claims 39-48, wherein the activator is a soluble ligand of the kinase, or a soluble ligand of a receptor that activates the kinase in vivo.

50. The method of any of claims 39-48, wherein the activator is an antibody or an antigen-binding fragment thereof that specifically binds to the kinase.

51. The method of claim 50, wherein the antibody is a monoclonal antibody.

52. The method of any of claims 39-51, wherein the immunosuppressive therapy is sirolimus, everolimus, rapamycin, one or more steroids, cyclosporine, cyclophosphamide, azathioprine, mercaptopurine, fluorouracil, fludarabine, interferon beta, a TNF decoy receptor, a TNF antibody, methotrexate, a T-cell antibody, an anti-CD20 antibody, a complement inhibitor, an anti-IL6 antibody, an anti-IL2R antibody, anti-thymocyte globulin, fingolimod, mycophenolate, or a combination thereof.

53. The method of claim 52, wherein the immunosuppressive therapy is a TNF decoy receptor that is etanercept.

54. The method of claim 52, wherein the immunosuppressive therapy is a TNF antibody that is infliximab.

55. The method of claim 52, wherein the immunosuppressive therapy is a T-cell antibody that is an anti-CD3 antibody.

56. The method of claim 55, wherein the anti-CD3 antibody is OKT3.

57. The method of claim 52, wherein the immunosuppressive therapy is an anti-CD20 antibody that is rituximab.

58. The method of claim 52, wherein the immunosuppressive therapy is a complement inhibitor that is eculizumab.

59. The method of claim 52, wherein the immunosuppressive therapy is an anti-IL2R antibody that is daclizumab.

60. The method of any of claims 1-59, wherein the patient is a human patient.


SEQUENCE LISTING (26 sequences)

SEQ ID NO: 1 - protein, 9 aa, Artificial Sequence
  peptide derived from WT1
RMFPNAPYL

SEQ ID NO: 2 - protein, 9 aa, Artificial Sequence
  HLA ligand
TLSGHSQEV

SEQ ID NO: 3 - protein, 9 aa, Artificial Sequence
  HLA ligand
VYSLIKNKI

SEQ ID NO: 4 - protein, 9 aa, Artificial Sequence
  HLA ligand
SYNEHWNYL

SEQ ID NO: 5 - protein, 9 aa, Artificial Sequence
  HLA ligand
ALSGLAVRL

SEQ ID NO: 6 - protein, 9 aa, Artificial Sequence
  control peptide
TYLEKAIKI

SEQ ID NO: 7 - protein, 9 aa, Artificial Sequence
  control peptide
ILDKKVEKV

SEQ ID NO: 8 - protein, 9 aa, Artificial Sequence
  control peptide
ILQAHLHSL

SEQ ID NO: 9 - protein, 9 aa, Artificial Sequence
  irrelevant peptide
GRKPPLLKK

SEQ ID NO: 10 - protein, 9 aa, Artificial Sequence
  off target peptide
RMFPGEVAL

SEQ ID NO: 11 - protein, 9 aa, Artificial Sequence
  TEIPP peptide found after RET inhibitor treatment
ALFSFVTAL

SEQ ID NO: 12 - protein, 9 aa, Artificial Sequence
  TEIPP peptide found after RET inhibitor treatment
FLGPWPAAS

SEQ ID NO: 13 - protein, 9 aa, Artificial Sequence
  TEIPP peptide found after RET inhibitor treatment
FLSELQYYL

SEQ ID NO: 14 - protein, 9 aa, Artificial Sequence
  TEIPP peptide found after RET inhibitor treatment
FLYPFLSHL

SEQ ID NO: 15 - protein, 9 aa, Artificial Sequence
  TEIPP peptide found after RET inhibitor treatment
ILEYLTAEV

SEQ ID NO: 16 - protein, 10 aa, Artificial Sequence
  TEIPP peptide found after RET inhibitor treatment
LLALAAGLAV

SEQ ID NO: 17 - protein, 10 aa, Artificial Sequence
  TEIPP peptide found after RET inhibitor treatment
LLLDVPTAAV

SEQ ID NO: 18 - protein, 10 aa, Artificial Sequence
  TEIPP peptide found after RET inhibitor treatment
LLLSAEPVPA

SEQ ID NO: 19 - protein, 9 aa, Artificial Sequence
  TEIPP peptide found after RET inhibitor treatment
LLWGRQLFA

SEQ ID NO: 20 - protein, 8 aa, Artificial Sequence
  TEIPP peptide found after RET inhibitor treatment
LSEKLERI

SEQ ID NO: 21 - protein, 10 aa, Artificial Sequence
  TEIPP peptide found after RET inhibitor treatment
LTLLGTLWGA

SEQ ID NO: 22 - protein, 10 aa, Artificial Sequence
  TEIPP peptide found after RET inhibitor treatment
SVLWLGALGL

SEQ ID NO: 23 - protein, 9 aa, Artificial Sequence
  TEIPP peptide found after RET inhibitor treatment
TLLGASLPA

SEQ ID NO: 24 - protein, 9 aa, Artificial Sequence
  TEIPP peptide found after RET inhibitor treatment
VIIKPLVWV

SEQ ID NO: 25 - protein, 9 aa, Artificial Sequence
  TEIPP peptide found after RET inhibitor treatment
VLAVFIKAV

SEQ ID NO: 26 - protein, 9 aa, Artificial Sequence
  TEIPP peptide found after RET inhibitor treatment
VLLDHLSLA